SEQUENCE LISTING (77 sequences)

SEQ ID NO: 1 - DNA, 71 bp, artificial sequence
  misc_feature (71)..(71): unsure
ggcggccgcaagcagtggtatcaacgcagagttaattaattttttttttttttttttttttttttttttvn

SEQ ID NO: 2 - DNA, 39 bp, artificial sequence
  primer
tcgtcggggaagcagtggtatcaacgcagagtattaatg

SEQ ID NO: 3 - DNA, 34 bp, artificial sequence
  primer
cattaatactctgcgttgataccactgcttcccc

SEQ ID NO: 4 - DNA, 23 bp, artificial sequence
  primer
aagcagtggtatcaacgcagagt

SEQ ID NO: 5 - DNA, 26 bp, artificial sequence
  primer
ctaccttttgctggaagtgtcatagc

SEQ ID NO: 6 - DNA, 25 bp, artificial sequence
  primer
cggccagggggcctatgttctttac

SEQ ID NO: 7 - DNA, 22 bp, artificial sequence
  primer
cgctgtctcaagcagcatctaa

SEQ ID NO: 8 - DNA, 609 bp, Chlamydomonas reinhardtii
atgcctgctaagaagaagaagaccggtgacggagtaaagaaggcgaagaagccaactccgccgctggaacctttcgactacaccccaccaaatatttacgagcttggggtgaaaccaagcttgtttgtgaggtgcagaatctgctgcaccgcgctgccgcacctgaactttgagtggccggcggtgccgacggagctgactctgggcgccatcaaggaggctgtggtgaagcggcacggcggcgccctgccggaactgtcgctgtacaaggacgtggtgcacccagagcactctctggcgggggcctcagatggggacacgctgaaggacattgggatagaaggtgtgcacatggctgagggcgtggcgctgccctccattaccttccactacgatttcgcggcggcccgctttgacgactccattgtggcggtggagcccgacccgctcgagtccaaacacgacctgtcgctgaagctgtaccgccgcctcacctcgccaagcgctgcgggggtggcggcggctgcggggaaaggatcggcagcgccagcgggagccggcgcggtggcaggcagtcgtgtgcttggctcgccgcttgcggcgaagtga

SEQ ID NO: 9 - DNA, 1149 bp, Chlamydomonas reinhardtii
atgcggtcccagctgctccgcttcctgacgcgggctcccgcaggcttcagccaggagggtctgcaggccctgcgcgctggcctgacctcgggcgaggcttccggtctgctgcagtcctctgccttcggccggcagaatgagagcgctgcgccccgcggcctgggcttcggcaagatggctctgcccctgagcttccagggccacctgatgagcaccctggcctccgccaacggtgacgacaagaaggagcccaccaccggcgccctggcgcagcagcctcaggtgcccaacgccctggcggccctgccccctcgcggcacccgcaccatgggcagccacgccgcgggtcaccagaccgccaaggagttctacatggagcacatcggcaagcgccaccccttccacgtgctgccgcccagcccctggcccatgctggccggctggggcacctacgtgagctgcctgggcatggccgcctggttccacaacatgcccaccggcggcgcgctgatggcgttcggcatggccaacatcgcctggaccgccatcacctggtggcgcgactgcgccattgagggcgacatgggcatgcacaccgaggtggtgcgcaagaacttcatcagcggcatgtgggccttcatcgtgtccgaggcgctgctgttcgtgggcctgctctgggcctgcctgcacctgggcatgtcgcccagcgtggcgctgcagatgcagtggccgcccgtgggcattgagcccatcggctgggacaagcgcgcgctggtcatgtcggcggtgctggccgcctcctactactcggccaacgtggccatggtggccaaggaccctaaggtggtgatgggcgcgctggccaccaccatcggcctgggcgccatgttcttggctgaccagtacctggagtacaacgagacacccttcaccatcaccgactcgccctacggcaccaccttcttcgtgaccaccggcttccacggcatgcacgtgctgctgggctcgctgtacctgaccgccgcgctcatgatgtacaagcgcacgcacaacgcgggcgccgcgctcaagagctccatcctgtactggcatttcgtcgacattgtgtggattgccgtgtacggcatcatctacgtcggccagtactag

SEQ ID NO: 10 - DNA, 510 bp, Chlamydomonas reinhardtii
atgcggcaccttttcgcactttccgcaattctacttatggcagtgagcctcacatgtgcggataagatgtcatggacgccctgcgatgccgggctcaccgagttcagccctagttcggttgtgctcacgccggacccgcccgtaattggctcgcccgcgacgtttgtgatctcgggtgacatcggcaacgagctgccgggcggtagtgtggacatgacggtgtccttctccgggctgcccatctactccagcacctcggacctgtgcaccaaaaccacctgcccggtaccggccggccccatcgccatcaccattgttcaggtgctgccgcccatcgcgccaccgggtgactacggtctgcaggtggtggcgcgcgggccggatggatccgagctggcgtgtgtcaacgtggcctttagcctggtgctgccctctgcggtgtcgggagcggcggggtcgcatggcgccgtgggccgtaagcagcgcatgggcagccgacaagtggcatga

SEQ ID NO: 11 - DNA, 933 bp, Chlamydomonas reinhardtii
atgcgctcagccgttctacaacgcggccaggcgcggcgagtgtcttgccgggttcgggcggatggttcgggcgtggattcgctgccctcgaccagcgccagcagcagcgcacgccctctcattgatcgccgtcagctcctgaccggtgctgctgcgtcggtcataaccttcgttggctgcccttgccccctgtgcaagcctggggaggcaaaggccgcagcttggaactatggcgaagttgcgggtccgccaacctggaagggtgtgtgtgcgacgggcaagcgccagtcgcccatcaacatcccgttgaacacatcggcgccgaaggtcgacgcggagatgggcgaattcgatttcgcctacggcagcttcgagaagtgcgacgtgctgaacacgggacacggcaccatgcaggtgaacttccccgctggcaacctggcgttcattggcaacatggagctggagctgctgcagttccacttccacgcgccctcggagcacgccatggatggccgccgttacgccatggaggcgcatctggtgcacaagaataaaagcaccggcaacctagctgtgctgggcattatgctggagcccggcggcctgatcaagaacccggcgctgtccactgctctggaggtggcgcccgaggtgcccctggccaagaagccctcgcccaagggcatcaaccccgtcatgctgctgcccaagaagagcaaggccgggacacggccgttcgtgcactaccctggctcgcttaccacgcccccgtgttcggagggggtggactggtttgtgttcatgcagcccatcaaggtgcccgacagccagatcctggacttcatgcgcttcgtgggcgacaacaagacatacgccaccaacacgcggccactgcagctgctcaacagccgcctggtcgaatacgagctgtga

SEQ ID NO: 12 - DNA, 1122 bp, Chlamydomonas reinhardtii
atgtctttgcaatcgtcaatccgtgccgactcaaactgcacgctgccaaacaaccctgtctgtgtacttttacctgttgactttattgtagcagccatggcctcgtctacttcctctgctatggccaagtgggccgcgcaagccgcgcggggctttgctgctgctgctccgagctcgggcaagggccgcaaggtcgccgtgctgggtgctgctggcggtattggccagcccctgtccatgctcatgaagatgaacagccaggtgtcgtctctgtcgctctacgatattgctggcacccccggtgtggccgctgatgtcagccacatcaacaccaaggcgcaggtcaagggcttcgacaaggacggcctggctgaggccctgcgtgggtgcgacctggtcatcatccccgccggtgtgccccgcaagcccggcatgacccgcgacgacctgttcaagatcaacgccggcatcgtgcgtgacctggtcaccgccgtgggccagcactgccccggggccgtgctgaacatcatctccaaccccgtcaacagcactgtgcccatcgcggccgagcagctgaagaagatgggcgtgtacgacaagcggaaggtgatgggcgtgaccaccctggacgtggtgcgtgccaagaccttctacgccgagaagaacggcctggacgtggccagcgtggacgtgcccgtggtgggcggccacgccggcgtgaccatcctgccgctgttctcccaggccacccccaaggccaccatgtcggctgaggtgctggacgcgctcaccaagcgtacccaggacggcggcactgaggtggtgcaggccaaggccggcaagggctcggccaccctgtccatggcctacgccgccgccctgttcgcggactcgtgcctgcgcggcctgaacggcgcccccgtggtggagtgcacctacgtggagtccaccgtcacggacgcgccctacttcgcctccaaggtcaagctcagcaccgagggtgtggacaagatccacgacctgggcccgctgtcggactacgagaaggccggcctcaaggccatgatgcccgagctgctggcgtcgatcgagaagggtgtgcagttcgtcaagggcgcctaa

SEQ ID NO: 13 - DNA, 393 bp, Chlamydomonas reinhardtii
atggcgtacattcttgatttcctctcgtacatgtgggagaagaacaatgacgactctgcttcctacaaggagtggcaggacttccttgatgtcgccgagaaggaccgccggcgcgatcgcatcatcgcgtggcgcgagcggaccatgtatgacgattacgctgcaaacgcggaggagcccatcatttacaagcgcacagccgggttcgtgtggcgcgaggtggaccccaccggccaggccaagaaccgcaaccacacgtacatgtacaacctgaagacgtttgggcaccgcagctacacccggcgcgacccctcggagctccaggcggcggccacctctgttgaggcgcgcgtcaagtccggcgagctgggcgggctgtcgccactggcgtaa

SEQ ID NO: 14 - DNA, 417 bp, Chlamydomonas reinhardtii
atgcgggcgctcgccttggcgctcctggcgctggctttgattgcgcagcaagcaaatgccaaggtcgctcgtggcgagttcatcagctgtcccggctgacgcctgaaccacctgccggaggttaagaagttcattcgaggggacatcgagaaggataaggcctacgagaacctcacagtgaacttcgtccccggcaggacccctgagctcgtgctctacgatgagaatggcgtggagattgagcgtatgttcgtggacaagctcaagtacgacgagctccacacgttagtgcagagcaagggcttcaagcggagagaccccaacgctgcagcggcaaccaccaccgctaccgcatccacacggcgtgcgcgcgcggctcgtcgccttctcagggcgacagaggagaccgcttgctag

SEQ ID NO: 15 - DNA, 603 bp, Chlamydomonas reinhardtii
atgcacgcacagtctcttcacgctaccgccaccaggacagctactagtgccaggcatgtggccccgcatgcgccgcgacgcaccgcttccgttcggtgctgtgcaagcatgccgacgagcggcgagactgcgccgagccggcggcaggcgctagcaagcattgcgctcatgagctctggtgctatgctgtcgaactgcgcgcccgcgaacgcggccctgaaggactacttgctgccaggccaggacgaggtgctcgaggtgtcgatgaccctgggcagcacggatggcaagtacatgttttcgccaagcaccctggagctcacgcagggcaagatttacaagctcaagctgaacaacccctcctccaccacccactacttcactgccctggagtttgccagcaagatttacacgctgctggtcctggcgggtgacccggtggtggaggtgaagggcgccatctcggaggtggctctcaaggctggcgccagcgccatctgggtgctcatgcccatcaagcccggcaagtacccgctgcgctgcaccgtcaagggccacaccgagggcggcatggtgggcaccatcatcgtcaacaaggcctga

SEQ ID NO: 16 - DNA, 993 bp, Chlamydomonas reinhardtii
atgccagcggggccagataaagttgtcttgcacccgctggtgctgctcagcgtggtcgaccactacaatcgggttgccaaggacacaaagaagcgcgtagtgggagtactattgggagagctttacaagggtcaaatagacgtcaccaacagctttgcgctgcccttcgaggaggacgaccatgacaccagcatctggttcctggaccactcctacttggagcagatgtacaaaatgttcaagaaagtgaacgctcgcgagaagatcgtgggctggtactccaccggccccaagctgcgcgagagcgacctggacatcaacgagctgatgcgcggcttctgcgagagccccgtgctggtcatctgcgaggtgcagcccaaggaggtggggctgcccttcaccgcctactacgcggtggacgaggtgcgcaccgacggcaccgagaaggccaagaaggtgttcaacagcctgcccaccgaggtcgggcagacggaggcggaggagatcggtgttgagcacctgctgcgggacgtcaaggacgccacgctgtcctcgctggcgggtgacgtcagctccaagctcatggcgctcaaggggctgcagggtcgcctgggcgagatcagcgagtacctgcagctggtgctggacggcaagctgcccaccaaccacgacgtcatcaacatcgtgcaggagatcttcaacctgctgcccaacatgaacgtggagtcgctgtccaagtcgctcgcggtcaagtccaacgacatgatgcacgtcatctacgtggccagcatggtgcgatccatcctggcgctgcacaagctcatcgacaacaaggaggggcgcctgtgggcggagaaggaggccgccaagaaggagaaggagaaggcggaggacaaggcgaagaaggaggccaaggagaaggaggagaaggagaaggcagacgccaaggccaaggcggacggcaaggcggaggacaaggacgggaaaaagtag

SEQ ID NO: 17 - DNA, 900 bp, Chlamydomonas reinhardtii
atgtcgaaccagagccagccccctgctgcttggaaggcggccaagccgttcgtgaacggtggcctgtccggcatgctggcgacttgcatcatccagcctattgacatggtcaaggtccgcatccagctgggcgccaagggcagcccgctggctgtcggcgctgagatcgtccgcaaggacggcgtcggtgccctgtacaagggcctgagcgccggtctgctgcgtcaggcgacctacacgaccactcggctgggtatcttcaacatgatgtcggaggagctcaaggcgcgcaacaacggccagaacctgcccctgtggcagaaggccgtggccggcctatcggctggtggcattggcgccctggtcggctcccccgccgatctgaccctgatccgcatgcaggccgacgccaccctgcccgttgagcagcgccgcaactacaagggcgttgctgacgccttcatccgtatcgtgaaggaggacggcgtaggtggcctgttccgcggtgccgcccccaccgtggtgcgcgccatgtcgctgaacatgggcatgctcgcctccaacgaccaggccaaggaggccatcgaggctgctggcttccccaagggtggcaccgtgtccgtgctgggtggcgccaccattgccggtttcatcgcttcggcattcagcctgccgttcgacttcatcaagactcgcctgcagaagatgacccccaaccccgacggcaccatgccgtacaagggccccatcgactgcgccctgcagacgctcaagaacgagggccccctcaagttctacactggcttcccgacctactgcatccgcattgcgccgcacgtcgtgttcacgctcgtgttcatggacgcgctgcccaaggtgcagaagaacttcggcctgtaa

SEQ ID NO: 18 - DNA, 747 bp, Chlamydomonas reinhardtii
atgatggccgcgccggtctcaacccagctgagccaggatgagctgaagaagcaggctgcctggaaggctgttgagtacgttaagagcggcatggtggttggcctgggcacgggctcgaccgctgcgttcgcggtggaccggatcggccagctgctgaaggagggcaagctgcagaacatcgtcggtgtgcccacctcgatccgcacctacgagcaggctctgtctctgggcattcctctggccactctggatgagcagcccaagctggacgtcgccatcgatggtgccgacgaggtggaccccaacctggacgtggtgaagggccgcggtggcgctcttctgcgcgaaaagatggtggagatggcctcggctaagtttgtttgcatcgtggacgactccaagctggtcgagggcctgggcggctccaagctggccatgccggtggagattgtgcagttctgccacaagtacaccctgcagcgcctggctaacctgcccgaggtgaagggctgcgaggctaagctgcgcatgaacggcgacaagccctacgtgaccgacaacagcaactacatcgtggacctgtacttccagacccctatcaaggattcccaggccgcctccaaggccatcctgggcctggacggtgtcgttgaccacggcctgttcctggacatggtggacgtgtgcatcatcgccggcgccaccggcgtcaccgtgcaggagcgccccaaccccaagaagcactaa

SEQ ID NO: 19 - DNA, 657 bp, Chlamydomonas reinhardtii
atgccgctgatttacagctcggtctcgcaagggaccgtaacgttggcagaatatgcggcgtttagtggaaattttggggctgtcgccaaagactacctggagaaggccgggaagaatgagggcaagttcaccttcaacgtggacggccacaccttcaacttcctcaaccgcggcggattcacctacctggtggttgctgatgaggcgtacgggcgagccatccccagcgccttcctggacaagatggcctcagagtttgccatgaagttcgcagacaaagccgcgggcgccaaggagggcggcctcaacggctcgttcggcaagcagctcaagtcaatgatggagcacgcgacgcagtacccggaggagtactccaaggtggcgtccgtgcagaagaaggtggatgaggtgaagggcatcatgacggagaacatcgagaaggtgctggcgcgcggcgagaagctggagctgctgaccgacaagaccgagaacctcatgaacgaggccgaccgcttccagcggacaggccgcacgttgcgccgcaagatgtggtggcagaactgcaagatgaagatcgtggtggctctggcggtcatcctgctggcggtggtcatcttcctgctggtctgcttctcgggcggcaactgcctcaagtaa

SEQ ID NO: 20 - DNA, 345 bp, Chlamydomonas reinhardtii
atggcgactcctcttgtggccggattgagtgtggcggcggcagctttcgtgggcaagcaggtggtgcagacctacatcaagtttaagacttctccaggcctgttcaactccgtggggcggcaatactacaagggcggcttcctgcctgagatgacgcggcgagaggcggcgctgatcctgggcatccgggagtctgcgggcgaggagcgcgtcaaggacgcccaccggcgaatcatggttgcgaaccaccccgattctggcggcagcagctacgtcgccgccaaggtcaacgaagcaaaggacttgttgttggggaagaagaaggtcggaaagtcacccttctaa

SEQ ID NO: 21 - DNA, 738 bp, Chlamydomonas reinhardtii
atgtcaccaacatatgctgagcatctgagcgtcatcaagggcttctgcgacaaggcagatggaaaggacaagcttaccgcgctcatacagtatgcctgcctcttcatttccgccggcgagcccggcaacatcaagaagatccaagcgtctgtgaccgccgcccggaaggtcttccgtgtcatgcggccactggagctcatgacgccgctgctgatcacgccgggcttcaccggcaagcagcccatgttgctggaggccgtcaacaaggtcaaggcggtgctcatggccgtgtacttcggtgccgatcacgtggtgtgggcgcaccagatcggcctcatcacagacaagaaggtcggcgagaggtaccagaagctgtcgctgtggtcttgggcgctgggcagcgtgtgcacggttgccacggagagctaccagattgcggccaagagcgtggtgcgcaaggagggcgagagcgaggaggactacgcgcggcgggtggaggaagttgaagaagcagatcaaccagcacctgtttgtgctcatccacggactgttccaggcagctctggcggcgggcctgctgcagctgctgccgctcaagccgcgcacggtgggcctgctgggcgtggtggcgtcggccatgaactgctacctgctgctgcccgcctaccccaagcccgtggccgctgccaaggccaagtcggcgtagaggcgtcgttaggcggccgtgcggcgttgtaa

SEQ ID NO: 22 - DNA, 435 bp, Chlamydomonas reinhardtii
atgcagagcagtcgctccatcggtgtggccagcacgtctggccgcgggagcagcagcagcagcctagcggccccccaacgcgtgcggacagggcgagctcgtgtggcccgcgtccaggcgttcgtggaccctgactccagtctggcgaccatccagaccgtctcccaggtcgttactacctgctgcattgcagccggcgctttcatgctcctgagcaagccaggggaatcgctcccagagcaggatcgcctggataaccgcaactcgcaaccgtgcccagtctgtggcggctcaggcttcgaggagtgcctctgctcgcgctggtcagatggcgacgtcgggtgcaactcgtgcagcaagacgggctacatgcggtgccgcggctgcgggggtggcggcacggctgtgcccctgatggtccgcgctcgcaagtga

SEQ ID NO: 23 - DNA, 651 bp, Chlamydomonas reinhardtii
atggccctgaccatgcgttcctctagccttgttaaggccagccgcggccgcctgactgtgtgcgcggctacccccgtgcccaagcagttcaaggccgtgaagcccgttggcgatcgcgtgcttgtcaaggttgacaaggaggaggcgaagagcgttggcggtgtgctgctgccggcgtccgtgcgcaacaagcccacggctggctcgattattgctctgggcgatgccaagagtgtcaaggtaggaggcacacacgtacacgcacacacgtacacacgcgctcacgccgactgctccacccgcgcgcacgtcctgcaggaggaggacgtgattggtgtgctgtccgccagcgagaagattgcgcagctcaagcccctgtccgaccgcatcctcatcaagggcgccaaggccgaggacaagacctcgggcggtgtgctgctggcgactgagtcggcggagaagcccaccttcggcaccgtggtggctgtgggcgagggccgcgaggacgaggagaccaaggcgctggtcaagcccaacgtgaccgtgggcgccaccgtcatgtactccaagtactcggggacagagttcgaggaggacggcgacaactacattgtggtgcgcgagagcgacatcctggcccagctctcgtaa

SEQ ID NO: 24 - DNA, 528 bp, Chlamydomonas reinhardtii
atgcagtcccttgcatcgcgcccctgcgtcctccgcgcgcagcgcccgttcaccgcgaagcgcagctcggtcgtccgcgtgcgtgccgaggcttcctcctcgtcgcctgtgcccatccccgtctggcctaaggacacagagagcgctcgcgatgtgtttgctttcggcggcagcgcgcccgagcgtgtgaacggccgcgtggccatgatgggcttcgtttccatcctggggcccgagctgtccaagaagcagccagtgctggagcaggtcggcgacgcctggtttggcatcctgctattctccctcaccatcaccttcgcctccatcctgcccaagctggtgtcgggtgtctccctgaaggagctgcactccgtggccacctctgagaacctgaagggcgagggcctgcagcaggcgctggcgctcttcgacaccaacgtggagctgtgggccggccgcctcgccatgctgggcttcgcgggcctgatcgccctggagaccattaagggcggcgaggctttcttctaa

SEQ ID NO: 25 - protein, 202 aa, Chlamydomonas reinhardtii
MPAKKKKTGDGVKKAKKPTPPLEPFDYTPPNIYELGVKPSLFVRCRICCTALPHLNFEWPAVPTELTLGAIKEAVVKRHGGALPELSLYKDVVHPEHSLAGASDGDTLKDIGIEGVHMAEGVALPSITFHYDFAAARFDDSIVAVEPDPLESKHDLSLKLYRRLTSPSAAGVAAAAGKGSAAPAGAGAVAGSRVLGSPLAAK

SEQ ID NO: 26 - protein, 382 aa, Chlamydomonas reinhardtii
MRSQLLRFLTRAPAGFSQEGLQALRAGLTSGEASGLLQSSAFGRQNESAAPRGLGFGKMALPLSFQGHLMSTLASANGDDKKEPTTGALAQQPQVPNALAALPPRGTRTMGSHAAGHQTAKEFYMEHIGKRHPFHVLPPSPWPMLAGWGTYVSCLGMAAWFHNMPTGGALMAFGMANIAWTAITWWRDCAIEGDMGMHTEVVRKNFISGMWAFIVSEALLFVGLLWACLHLGMSPSVALQMQWPPVGIEPIGWDKRALVMSAVLAASYYSANVAMVAKDPKVVMGALATTIGLGAMFLADQYLEYNETPFTITDSPYGTTFFVTTGFHGMHVLLGSLYLTAALMMYKRTHNAGAALKSSILYWHFVDIVWIAVYGIIYVGQY

SEQ ID NO: 27 - protein, 169 aa, Chlamydomonas reinhardtii
MRHLFALSAILLMAVSLTCADKMSWTPCDAGLTEFSPSSVVLTPDPPVIGSPATFVISGDIGNELPGGSVDMTVSFSGLPIYSSTSDLCTKTTCPVPAGPIAITIVQVLPPIAPPGDYGLQVVARGPDGSELACVNVAFSLVLPSAVSGAAGSHGAVGRKQRMGSRQVA

SEQ ID NO: 28 - protein, 310 aa, Chlamydomonas reinhardtii
MRSAVLQRGQARRVSCRVRADGSGVDSLPSTSASSSARPLIDRRQLLTGAAASVITFVGCPCPLCKPGEAKAAAWNYGEVAGPPTWKGVCATGKRQSPINIPLNTSAPKVDAEMGEFDFAYGSFEKCDVLNTGHGTMQVNFPAGNLAFIGNMELELLQFHFHAPSEHAMDGRRYAMEAHLVHKNKSTGNLAVLGIMLEPGGLIKNPALSTALEVAPEVPLAKKPSPKGINPVMLLPKKSKAGTRPFVHYPGSLTTPPCSEGVDWFVFMQPIKVPDSQILDFMRFVGDNKTYATNTRPLQLLNSRLVEYEL

SEQ ID NO: 29 - protein, 373 aa, Chlamydomonas reinhardtii
MSLQSSIRADSNCTLPNNPVCVLLPVDFIVAAMASSTSSAMAKWAAQAARGFAAAAPSSGKGRKVAVLGAAGGIGQPLSMLMKMNSQVSSLSLYDIAGTPGVAADVSHINTKAQVKGFDKDGLAEALRGCDLVIIPAGVPRKPGMTRDDLFKINAGIVRDLVTAVGQHCPGAVLNIISNPVNSTVPIAAEQLKKMGVYDKRKVMGVTTLDVVRAKTFYAEKNGLDVASVDVPVVGGHAGVTILPLFSQATPKATMSAEVLDALTKRTQDGGTEVVQAKAGKGSATLSMAYAAALFADSCLRGLNGAPVVECTYVESTVTDAPYFASKVKLSTEGVDKIHDLGPLSDYEKAGLKAMMPELLASIEKGVQFVKGA

SEQ ID NO: 30 - protein, 130 aa, Chlamydomonas reinhardtii
MAYILDFLSYMWEKNNDDSASYKEWQDFLDVAEKDRRRDRIIAWRERTMYDDYAANAEEPIIYKRTAGFVWREVDPTGQAKNRNHTYMYNLKTFGHRSYTRRDPSELQAAATSVEARVKSGELGGLSPLA

SEQ ID NO: 31 - protein, 138 aa, Chlamydomonas reinhardtii
  MISC_FEATURE (33)..(33): Xaa=Selenocysteine
MRALALALLALALIAQQANAKVARGEFISCPGXRLNHLPEVKKFIRGDIEKDKAYENLTVNFVPGRTPELVLYDENGVEIERMFVDKLKYDELHTLVQSKGFKRRDPNAAAATTTATASTRRARAARRLLRATEETAC

SEQ ID NO: 32 - protein, 200 aa, Chlamydomonas reinhardtii
MHAQSLHATATRTATSARHVAPHAPRRTASVRCCASMPTSGETAPSRRQALASIALMSSGAMLSNCAPANAALKDYLLPGQDEVLEVSMTLGSTDGKYMFSPSTLELTQGKIYKLKLNNPSSTTHYFTALEFASKIYTLLVLAGDPVVEVKGAISEVALKAGASAIWVLMPIKPGKYPLRCTVKGHTEGGMVGTIIVNKA

SEQ ID NO: 33 - protein, 330 aa, Chlamydomonas reinhardtii
MPAGPDKVVLHPLVLLSVVDHYNRVAKDTKKRVVGVLLGELYKGQIDVTNSFALPFEEDDHDTSIWFLDHSYLEQMYKMFKKVNAREKIVGWYSTGPKLRESDLDINELMRGFCESPVLVICEVQPKEVGLPFTAYYAVDEVRTDGTEKAKKVFNSLPTEVGQTEAEEIGVEHLLRDVKDATLSSLAGDVSSKLMALKGLQGRLGEISEYLQLVLDGKLPTNHDVINIVQEIFNLLPNMNVESLSKSLAVKSNDMMHVIYVASMVRSILALHKLIDNKEGRLWAEKEAAKKEKEKAEDKAKKEAKEKEEKEKADAKAKADGKAEDKDGKK

SEQ ID NO: 34 - protein, 299 aa, Chlamydomonas reinhardtii
MSNQSQPPAAWKAAKPFVNGGLSGMLATCIIQPIDMVKVRIQLGAKGSPLAVGAEIVRKDGVGALYKGLSAGLLRQATYTTTRLGIFNMMSEELKARNNGQNLPLWQKAVAGLSAGGIGALVGSPADLTLIRMQADATLPVEQRRNYKGVADAFIRIVKEDGVGGLFRGAAPTVVRAMSLNMGMLASNDQAKEAIEAAGFPKGGTVSVLGGATIAGFIASAFSLPFDFIKTRLQKMTPNPDGTMPYKGPIDCALQTLKNEGPLKFYTGFPTYCIRIAPHVVFTLVFMDALPKVQKNFGL

SEQ ID NO: 35 - protein, 248 aa, Chlamydomonas reinhardtii
MMAAPVSTQLSQDELKKQAAWKAVEYVKSGMVVGLGTGSTAAFAVDRIGQLLKEGKLQNIVGVPTSIRTYEQALSLGIPLATLDEQPKLDVAIDGADEVDPNLDVVKGRGGALLREKMVEMASAKFVCIVDDSKLVEGLGGSKLAMPVEIVQFCHKYTLQRLANLPEVKGCEAKLRMNGDKPYVTDNSNYIVDLYFQTPIKDSQAASKAILGLDGVVDHGLFLDMVDVCIIAGATGVTVQERPNPKKH

SEQ ID NO: 36 - protein, 218 aa, Chlamydomonas reinhardtii
MPLIYSSVSQGTVTLAEYAAFSGNFGAVAKDYLEKAGKNEGKFTFNVDGHTFNFLNRGGFTYLVVADEAYGRAIPSAFLDKMASEFAMKFADKAAGAKEGGLNGSFGKQLKSMMEHATQYPEEYSKVASVQKKVDEVKGIMTENIEKVLARGEKLELLTDKTENLMNEADRFQRTGRTLRRKMWWQNCKMKIVVALAVILLAVVIFLLVCFSGGNCLK

SEQ ID NO: 37 - protein, 114 aa, Chlamydomonas reinhardtii
MATPLVAGLSVAAAAFVGKQVVQTYIKFKTSPGLFNSVGRQYYKGGFLPEMTRREAALILGIRESAGEERVKDAHRRIMVANHPDSGGSSYVAAKVNEAKDLLLGKKKVGKSPF

SEQ ID NO: 38 - protein, 245 aa, Chlamydomonas reinhardtii
MSPTYAEHLSVIKGFCDKADGKDKLTALIQYACLFISAGEPGNIKKIQASVTAARKVFRVMRPLELMTPLLITPGFTGKQPMLLEAVNKVKAVLMAVYFGADHVVWAHQIGLITDKKVGERYQKLSLWSWALGSVCTVATESYQIAAKSVVRKEGESEEDYARRVEEVEEADQPAPVCAHPRTVPGSSGGGPAAAAAAQAAHGGPAGRGGVGHELLPAAARLPQARGRCQGQVGVEASLGGRAAL

SEQ ID NO: 39 - protein, 144 aa, Chlamydomonas reinhardtii
MQSSRSIGVASTSGRGSSSSSLAAPQRVRTGRARVARVQAFVDPDSSLATIQTVSQVVTTCCIAAGAFMLLSKPGESLPEQDRLDNRNSQPCPVCGGSGFEECLCSRWSDGDVGCNSCSKTGYMRCRGCGGGGTAVPLMVRARK

SEQ ID NO: 40 - protein, 216 aa, Chlamydomonas reinhardtii
MALTMRSSSLVKASRGRLTVCAATPVPKQFKAVKPVGDRVLVKVDKEEAKSVGGVLLPASVRNKPTAGSIIALGDAKSVKVGGTHVHAHTYTRAHADCSTRAHVLQEEDVIGVLSASEKIAQLKPLSDRILIKGAKAEDKTSGGVLLATESAEKPTFGTVVAVGEGREDEETKALVKPNVTVGATVMYSKYSGTEFEEDGDNYIVVRESDILAQLS

SEQ ID NO: 41 - protein, 175 aa, Chlamydomonas reinhardtii
MQSLASRPCVLRAQRPFTAKRSSVVRVRAEASSSSPVPIPVWPKDTESARDVFAFGGSAPERVNGRVAMMGFVSILGPELSKKQPVLEQVGDAWFGILLFSLTITFASILPKLVSGVSLKELHSVATSENLKGEGLQQALALFDTNVELWAGRLAMLGFAGLIALETIKGGEAFF

SEQ ID NO: 42 - DNA, 603 bp, Chlamydomonas reinhardtii
cctgctaagaagaagaagaccggtgacggagtaaagaaggcgaagaagccaactccgccgctggaacctttcgactacaccccaccaaatatttacgagcttggggtgaaaccaagcttgtttgtgaggtgcagaatctgctgcaccgcgctgccgcacctgaactttgagtggccggcggtgccgacggagctgactctgggcgccatcaaggaggctgtggtgaagcggcacggcggcgccctgccggaactgtcgctgtacaaggacgtggtgcacccagagcactctctggcgggggcctcagatggggacacgctgaaggacattgggatagaaggtgtgcacatggctgagggcgtggcgctgccctccattaccttccactacgatttcgcggcggcccgctttgacgactccattgtggcggtggagcccgacccgctcgagtccaaacacgacctgtcgctgaagctgtaccgccgcctcacctcgccaagcgctgcgggggtggcggcggctgcggggaaaggatcggcagcgccagcgggagccggcgcggtggcaggcagtcgtgtgcttggctcgccgcttgcggcgaag

SEQ ID NO: 43 - protein, 201 aa, Chlamydomonas reinhardtii
PAKKKKTGDGVKKAKKPTPPLEPFDYTPPNIYELGVKPSLFVRCRICCTALPHLNFEWPAVPTELTLGAIKEAVVKRHGGALPELSLYKDVVHPEHSLAGASDGDTLKDIGIEGVHMAEGVALPSITFHYDFAAARFDDSIVAVEPDPLESKHDLSLKLYRRLTSPSAAGVAAAAGKGSAAPAGAGAVAGSRVLGSPLAAK

SEQ ID NO: 44 - DNA, 1143 bp, Chlamydomonas reinhardtii
cggtcccagctgctccgcttcctgacgcgggctcccgcaggcttcagccaggagggtctgcaggccctgcgcgctggcctgacctcgggcgaggcttccggtctgctgcagtcctctgccttcggccggcagaatgagagcgctgcgccccgcggcctgggcttcggcaagatggctctgcccctgagcttccagggccacctgatgagcaccctggcctccgccaacggtgacgacaagaaggagcccaccaccggcgccctggcgcagcagcctcaggtgcccaacgccctggcggccctgccccctcgcggcacccgcaccatgggcagccacgccgcgggtcaccagaccgccaaggagttctacatggagcacatcggcaagcgccaccccttccacgtgctgccgcccagcccctggcccatgctggccggctggggcacctacgtgagctgcctgggcatggccgcctggttccacaacatgcccaccggcggcgcgctgatggcgttcggcatggccaacatcgcctggaccgccatcacctggtggcgcgactgcgccattgagggcgacatgggcatgcacaccgaggtggtgcgcaagaacttcatcagcggcatgtgggccttcatcgtgtccgaggcgctgctgttcgtgggcctgctctgggcctgcctgcacctgggcatgtcgcccagcgtggcgctgcagatgcagtggccgcccgtgggcattgagcccatcggctgggacaagcgcgcgctggtcatgtcggcggtgctggccgcctcctactactcggccaacgtggccatggtggccaaggaccctaaggtggtgatgggcgcgctggccaccaccatcggcctgggcgccatgttcttggctgaccagtacctggagtacaacgagacacccttcaccatcaccgactcgccctacggcaccaccttcttcgtgaccaccggcttccacggcatgcacgtgctgctgggctcgctgtacctgaccgccgcgctcatgatgtacaagcgcacgcacaacgcgggcgccgcgctcaagagctccatcctgtactggcatttcgtcgacattgtgtggattgccgtgtacggcatcatctacgtcggccagtac

SEQ ID NO: 45 - protein, 381 aa, Chlamydomonas reinhardtii
RSQLLRFLTRAPAGFSQEGLQALRAGLTSGEASGLLQSSAFGRQNESAAPRGLGFGKMALPLSFQGHLMSTLASANGDDKKEPTTGALAQQPQVPNALAALPPRGTRTMGSHAAGHQTAKEFYMEHIGKRHPFHVLPPSPWPMLAGWGTYVSCLGMAAWFHNMPTGGALMAFGMANIAWTAITWWRDCAIEGDMGMHTEVVRKNFISGMWAFIVSEALLFVGLLWACLHLGMSPSVALQMQWPPVGIEPIGWDKRALVMSAVLAASYYSANVAMVAKDPKVVMGALATTIGLGAMFLADQYLEYNETPFTITDSPYGTTFFVTTGFHGMHVLLGSLYLTAALMMYKRTHNAGAALKSSILYWHFVDIVWIAVYGIIYVGQY

SEQ ID NO: 46 - DNA, 504 bp, Chlamydomonas reinhardtii
cggcaccttttcgcactttccgcaattctacttatggcagtgagcctcacatgtgcggataagatgtcatggacgccctgcgatgccgggctcaccgagttcagccctagttcggttgtgctcacgccggacccgcccgtaattggctcgcccgcgacgtttgtgatctcgggtgacatcggcaacgagctgccgggcggtagtgtggacatgacggtgtccttctccgggctgcccatctactccagcacctcggacctgtgcaccaaaaccacctgcccggtaccggccggccccatcgccatcaccattgttcaggtgctgccgcccatcgcgccaccgggtgactacggtctgcaggtggtggcgcgcgggccggatggatccgagctggcgtgtgtcaacgtggcctttagcctggtgctgccctctgcggtgtcgggagcggcggggtcgcatggcgccgtgggccgtaagcagcgcatgggcagccgacaagtggca

SEQ ID NO: 47 - protein, 168 aa, Chlamydomonas reinhardtii
RHLFALSAILLMAVSLTCADKMSWTPCDAGLTEFSPSSVVLTPDPPVIGSPATFVISGDIGNELPGGSVDMTVSFSGLPIYSSTSDLCTKTTCPVPAGPIAITIVQVLPPIAPPGDYGLQVVARGPDGSELACVNVAFSLVLPSAVSGAAGSHGAVGRKQRMGSRQVA

SEQ ID NO: 48 - DNA, 927 bp, Chlamydomonas reinhardtii
cgctcagccgttctacaacgcggccaggcgcggcgagtgtcttgccgggttcgggcggatggttcgggcgtggattcgctgccctcgaccagcgccagcagcagcgcacgccctctcattgatcgccgtcagctcctgaccggtgctgctgcgtcggtcataaccttcgttggctgcccttgccccctgtgcaagcctggggaggcaaaggccgcagcttggaactatggcgaagttgcgggtccgccaacctggaagggtgtgtgtgcgacgggcaagcgccagtcgcccatcaacatcccgttgaacacatcggcgccgaaggtcgacgcggagatgggcgaattcgatttcgcctacggcagcttcgagaagtgcgacgtgctgaacacgggacacggcaccatgcaggtgaacttccccgctggcaacctggcgttcattggcaacatggagctggagctgctgcagttccacttccacgcgccctcggagcacgccatggatggccgccgttacgccatggaggcgcatctggtgcacaagaataaaagcaccggcaacctagctgtgctgggcattatgctggagcccggcggcctgatcaagaacccggcgctgtccactgctctggaggtggcgcccgaggtgcccctggccaagaagccctcgcccaagggcatcaaccccgtcatgctgctgcccaagaagagcaaggccgggacacggccgttcgtgcactaccctggctcgcttaccacgcccccgtgttcggagggggtggactggtttgtgttcatgcagcccatcaaggtgcccgacagccagatcctggacttcatgcgcttcgtgggcgacaacaagacatacgccaccaacacgcggccactgcagctgctcaacagccgcctggtcgaatacgagctg

SEQ ID NO: 49 - protein, 309 aa, Chlamydomonas reinhardtii
RSAVLQRGQARRVSCRVRADGSGVDSLPSTSASSSARPLIDRRQLLTGAAASVITFVGCPCPLCKPGEAKAAAWNYGEVAGPPTWKGVCATGKRQSPINIPLNTSAPKVDAEMGEFDFAYGSFEKCDVLNTGHGTMQVNFPAGNLAFIGNMELELLQFHFHAPSEHAMDGRRYAMEAHLVHKNKSTGNLAVLGIMLEPGGLIKNPALSTALEVAPEVPLAKKPSPKGINPVMLLPKKSKAGTRPFVHYPGSLTTPPCSEGVDWFVFMQPIKVPDSQILDFMRFVGDNKTYATNTRPLQLLNSRLVEYEL

SEQ ID NO: 50 - DNA, 1116 bp, Chlamydomonas reinhardtii
tctttgcaatcgtcaatccgtgccgactcaaactgcacgctgccaaacaaccctgtctgtgtacttttacctgttgactttattgtagcagccatggcctcgtctacttcctctgctatggccaagtgggccgcgcaagccgcgcggggctttgctgctgctgctccgagctcgggcaagggccgcaaggtcgccgtgctgggtgctgctggcggtattggccagcccctgtccatgctcatgaagatgaacagccaggtgtcgtctctgtcgctctacgatattgctggcacccccggtgtggccgctgatgtcagccacatcaacaccaaggcgcaggtcaagggcttcgacaaggacggcctggctgaggccctgcgtgggtgcgacctggtcatcatccccgccggtgtgccccgcaagcccggcatgacccgcgacgacctgttcaagatcaacgccggcatcgtgcgtgacctggtcaccgccgtgggccagcactgccccggggccgtgctgaacatcatctccaaccccgtcaacagcactgtgcccatcgcggccgagcagctgaagaagatgggcgtgtacgacaagcggaaggtgatgggcgtgaccaccctggacgtggtgcgtgccaagaccttctacgccgagaagaacggcctggacgtggccagcgtggacgtgcccgtggtgggcggccacgccggcgtgaccatcctgccgctgttctcccaggccacccccaaggccaccatgtcggctgaggtgctggacgcgctcaccaagcgtacccaggacggcggcactgaggtggtgcaggccaaggccggcaagggctcggccaccctgtccatggcctacgccgccgccctgttcgcggactcgtgcctgcgcggcctgaacggcgcccccgtggtggagtgcacctacgtggagtccaccgtcacggacgcgccctacttcgcctccaaggtcaagctcagcaccgagggtgtggacaagatccacgacctgggcccgctgtcggactacgagaaggccggcctcaaggccatgatgcccgagctgctggcgtcgatcgagaagggtgtgcagttcgtcaagggcgcc

SEQ ID NO: 51 - protein, 372 aa, Chlamydomonas reinhardtii
SLQSSIRADSNCTLPNNPVCVLLPVDFIVAAMASSTSSAMAKWAAQAARGFAAAAPSSGKGRKVAVLGAAGGIGQPLSMLMKMNSQVSSLSLYDIAGTPGVAADVSHINTKAQVKGFDKDGLAEALRGCDLVIIPAGVPRKPGMTRDDLFKINAGIVRDLVTAVGQHCPGAVLNIISNPVNSTVPIAAEQLKKMGVYDKRKVMGVTTLDVVRAKTFYAEKNGLDVASVDVPVVGGHAGVTILPLFSQATPKATMSAEVLDALTKRTQDGGTEVVQAKAGKGSATLSMAYAAALFADSCLRGLNGAPVVECTYVESTVTDAPYFASKVKLSTEGVDKIHDLGPLSDYEKAGLKAMMPELLASIEKGVQFVKGA

SEQ ID NO: 52 - DNA, 387 bp, Chlamydomonas reinhardtii
gcgtacattcttgatttcctctcgtacatgtgggagaagaacaatgacgactctgcttcctacaaggagtggcaggacttccttgatgtcgccgagaaggaccgccggcgcgatcgcatcatcgcgtggcgcgagcggaccatgtatgacgattacgctgcaaacgcggaggagcccatcatttacaagcgcacagccgggttcgtgtggcgcgaggtggaccccaccggccaggccaagaaccgcaaccacacgtacatgtacaacctgaagacgtttgggcaccgcagctacacccggcgcgacccctcggagctccaggcggcggccacctctgttgaggcgcgcgtcaagtccggcgagctgggcgggctgtcgccactggcg

SEQ ID NO: 53 - protein, 129 aa, Chlamydomonas reinhardtii
AYILDFLSYMWEKNNDDSASYKEWQDFLDVAEKDRRRDRIIAWRERTMYDDYAANAEEPIIYKRTAGFVWREVDPTGQAKNRNHTYMYNLKTFGHRSYTRRDPSELQAAATSVEARVKSGELGGLSPLA

SEQ ID NO: 54 - DNA, 411 bp, Chlamydomonas reinhardtii
cgggcgctcgccttggcgctcctggcgctggctttgattgcgcagcaagcaaatgccaaggtcgctcgtggcgagttcatcagctgtcccggctgacgcctgaaccacctgccggaggttaagaagttcattcgaggggacatcgagaaggataaggcctacgagaacctcacagtgaacttcgtccccggcaggacccctgagctcgtgctctacgatgagaatggcgtggagattgagcgtatgttcgtggacaagctcaagtacgacgagctccacacgttagtgcagagcaagggcttcaagcggagagaccccaacgctgcagcggcaaccaccaccgctaccgcatccacacggcgtgcgcgcgcggctcgtcgccttctcagggcgacagaggagaccgcttgc

SEQ ID NO: 55 - protein, 137 aa, Chlamydomonas reinhardtii
  MISC_FEATURE (32)..(32): X=Selenocysteine
RALALALLALALIAQQANAKVARGEFISCPGXRLNHLPEVKKFIRGDIEKDKAYENLTVNFVPGRTPELVLYDENGVEIERMFVDKLKYDELHTLVQSKGFKRRDPNAAAATTTATASTRRARAARRLLRATEETAC

SEQ ID NO: 56 - DNA, 597 bp, Chlamydomonas reinhardtii
cacgcacagtctcttcacgctaccgccaccaggacagctactagtgccaggcatgtggccccgcatgcgccgcgacgcaccgcttccgttcggtgctgtgcaagcatgccgacgagcggcgagactgcgccgagccggcggcaggcgctagcaagcattgcgctcatgagctctggtgctatgctgtcgaactgcgcgcccgcgaacgcggccctgaaggactacttgctgccaggccaggacgaggtgctcgaggtgtcgatgaccctgggcagcacggatggcaagtacatgttttcgccaagcaccctggagctcacgcagggcaagatttacaagctcaagctgaacaacccctcctccaccacccactacttcactgccctggagtttgccagcaagatttacacgctgctggtcctggcgggtgacccggtggtggaggtgaagggcgccatctcggaggtggctctcaaggctggcgccagcgccatctgggtgctcatgcccatcaagcccggcaagtacccgctgcgctgcaccgtcaagggccacaccgagggcggcatggtgggcaccatcatcgtcaacaaggcc

SEQ ID NO: 57 - protein, 199 aa, Chlamydomonas reinhardtii
HAQSLHATATRTATSARHVAPHAPRRTASVRCCASMPTSGETAPSRRQALASIALMSSGAMLSNCAPANAALKDYLLPGQDEVLEVSMTLGSTDGKYMFSPSTLELTQGKIYKLKLNNPSSTTHYFTALEFASKIYTLLVLAGDPVVEVKGAISEVALKAGASAIWVLMPIKPGKYPLRCTVKGHTEGGMVGTIIVNKA

SEQ ID NO: 58 - DNA, 987 bp, Chlamydomonas reinhardtii
ccagcggggccagataaagttgtcttgcacccgctggtgctgctcagcgtggtcgaccactacaatcgggttgccaaggacacaaagaagcgcgtagtgggagtactattgggagagctttacaagggtcaaatagacgtcaccaacagctttgcgctgcccttcgaggaggacgaccatgacaccagcatctggttcctggaccactcctacttggagcagatgtacaaaatgttcaagaaagtgaacgctcgcgagaagatcgtgggctggtactccaccggccccaagctgcgcgagagcgacctggacatcaacgagctgatgcgcggcttctgcgagagccccgtgctggtcatctgcgaggtgcagcccaaggaggtggggctgcccttcaccgcctactacgcggtggacgaggtgcgcaccgacggcaccgagaaggccaagaaggtgttcaacagcctgcccaccgaggtcgggcagacggaggcggaggagatcggtgttgagcacctgctgcgggacgtcaaggacgccacgctgtcctcgctggcgggtgacgtcagctccaagctcatggcgctcaaggggctgcagggtcgcctgggcgagatcagcgagtacctgcagctggtgctggacggcaagctgcccaccaaccacgacgtcatcaacatcgtgcaggagatcttcaacctgctgcccaacatgaacgtggagtcgctgtccaagtcgctcgcggtcaagtccaacgacatgatgcacgtcatctacgtggccagcatggtgcgatccatcctggcgctgcacaagctcatcgacaacaaggaggggcgcctgtgggcggagaaggaggccgccaagaaggagaaggagaaggcggaggacaaggcgaagaaggaggccaaggagaaggaggagaaggagaaggcagacgccaaggccaaggcggacggcaaggcggaggacaaggacgggaaaaag

SEQ ID NO: 59 - protein, 329 aa, Chlamydomonas reinhardtii
PAGPDKVVLHPLVLLSVVDHYNRVAKDTKKRVVGVLLGELYKGQIDVTNSFALPFEEDDHDTSIWFLDHSYLEQMYKMFKKVNAREKIVGWYSTGPKLRESDLDINELMRGFCESPVLVICEVQPKEVGLPFTAYYAVDEVRTDGTEKAKKVFNSLPTEVGQTEAEEIGVEHLLRDVKDATLSSLAGDVSSKLMALKGLQGRLGEISEYLQLVLDGKLPTNHDVINIVQEIFNLLPNMNVESLSKSLAVKSNDMMHVIYVASMVRSILALHKLIDNKEGRLWAEKEAAKKEKEKAEDKAKKEAKEKEEKEKADAKAKADGKAEDKDGKK

SEQ ID NO: 60 - DNA, 894 bp, Chlamydomonas reinhardtii
tcgaaccagagccagccccctgctgcttggaaggcggccaagccgttcgtgaacggtggcctgtccggcatgctggcgacttgcatcatccagcctattgacatggtcaaggtccgcatccagctgggcgccaagggcagcccgctggctgtcggcgctgagatcgtccgcaaggacggcgtcggtgccctgtacaagggcctgagcgccggtctgctgcgtcaggcgacctacacgaccactcggctgggtatcttcaacatgatgtcggaggagctcaaggcgcgcaacaacggccagaacctgcccctgtggcagaaggccgtggccggcctatcggctggtggcattggcgccctggtcggctcccccgccgatctgaccctgatccgcatgcaggccgacgccaccctgcccgttgagcagcgccgcaactacaagggcgttgctgacgccttcatccgtatcgtgaaggaggacggcgtaggtggcctgttccgcggtgccgcccccaccgtggtgcgcgccatgtcgctgaacatgggcatgctcgcctccaacgaccaggccaaggaggccatcgaggctgctggcttccccaagggtggcaccgtgtccgtgctgggtggcgccaccattgccggtttcatcgcttcggcattcagcctgccgttcgacttcatcaagactcgcctgcagaagatgacccccaaccccgacggcaccatgccgtacaagggccccatcgactgcgccctgcagacgctcaagaacgagggccccctcaagttctacactggcttcccgacctactgcatccgcattgcgccgcacgtcgtgttcacgctcgtgttcatggacgcgctgcccaaggtgcagaagaacttcggcctg

SEQ ID NO: 61 - protein, 298 aa, Chlamydomonas reinhardtii
SNQSQPPAAWKAAKPFVNGGLSGMLATCIIQPIDMVKVRIQLGAKGSPLAVGAEIVRKDGVGALYKGLSAGLLRQATYTTTRLGIFNMMSEELKARNNGQNLPLWQKAVAGLSAGGIGALVGSPADLTLIRMQADATLPVEQRRNYKGVADAFIRIVKEDGVGGLFRGAAPTVVRAMSLNMGMLASNDQAKEAIEAAGFPKGGTVSVLGGATIAGFIASAFSLPFDFIKTRLQKMTPNPDGTMPYKGPIDCALQTLKNEGPLKFYTGFPTYCIRIAPHVVFTLVFMDALPKVQKNFGL

SEQ ID NO: 62 - DNA, 741 bp, Chlamydomonas reinhardtii
atggccgcgccggtctcaacccagctgagccaggatgagctgaagaagcaggctgcctggaaggctgttgagtacgttaagagcggcatggtggttggcctgggcacgggctcgaccgctgcgttcgcggtggaccggatcggccagctgctgaaggagggcaagctgcagaacatcgtcggtgtgcccacctcgatccgcacctacgagcaggctctgtctctgggcattcctctggccactctggatgagcagcccaagctggacgtcgccatcgatggtgccgacgaggtggaccccaacctggacgtggtgaagggccgcggtggcgctcttctgcgcgaaaagatggtggagatggcctcggctaagtttgtttgcatcgtggacgactccaagctggtcgagggcctgggcggctccaagctggccatgccggtggagattgtgcagttctgccacaagtacaccctgcagcgcctggctaacctgcccgaggtgaagggctgcgaggctaagctgcgcatgaacggcgacaagccctacgtgaccgacaacagcaactacatcgtggacctgtacttccagacccctatcaaggattcccaggccgcctccaaggccatcctgggcctggacggtgtcgttgaccacggcctgttcctggacatggtggacgtgtgcatcatcgccggcgccaccggcgtcaccgtgcaggagcgccccaaccccaagaagcac

SEQ ID NO: 63 - protein, 247 aa, Chlamydomonas reinhardtii
MAAPVSTQLSQDELKKQAAWKAVEYVKSGMVVGLGTGSTAAFAVDRIGQLLKEGKLQNIVGVPTSIRTYEQALSLGIPLATLDEQPKLDVAIDGADEVDPNLDVVKGRGGALLREKMVEMASAKFVCIVDDSKLVEGLGGSKLAMPVEIVQFCHKYTLQRLANLPEVKGCEAKLRMNGDKPYVTDNSNYIVDLYFQTPIKDSQAASKAILGLDGVVDHGLFLDMVDVCIIAGATGVTVQERPNPKKH

SEQ ID NO: 64 - DNA, 651 bp, Chlamydomonas reinhardtii
ccgctgatttacagctcggtctcgcaagggaccgtaacgttggcagaatatgcggcgtttagtggaaattttggggctgtcgccaaagactacctggagaaggccgggaagaatgagggcaagttcaccttcaacgtggacggccacaccttcaacttcctcaaccgcggcggattcacctacctggtggttgctgatgaggcgtacgggcgagccatccccagcgccttcctggacaagatggcctcagagtttgccatgaagttcgcagacaaagccgcgggcgccaaggagggcggcctcaacggctcgttcggcaagcagctcaagtcaatgatggagcacgcgacgcagtacccggaggagtactccaaggtggcgtccgtgcagaagaaggtggatgaggtgaagggcatcatgacggagaacatcgagaaggtgctggcgcgcggcgagaagctggagctgctgaccgacaagaccgagaacctcatgaacgaggccgaccgcttccagcggacaggccgcacgttgcgccgcaagatgtggtggcagaactgcaagatgaagatcgtggtggctctggcggtcatcctgctggcggtggtcatcttcctgctggtctgcttctcgggcggcaactgcctcaag

SEQ ID NO: 65 - protein, 217 aa, Chlamydomonas reinhardtii
PLIYSSVSQGTVTLAEYAAFSGNFGAVAKDYLEKAGKNEGKFTFNVDGHTFNFLNRGGFTYLVVADEAYGRAIPSAFLDKMASEFAMKFADKAAGAKEGGLNGSFGKQLKSMMEHATQYPEEYSKVASVQKKVDEVKGIMTENIEKVLARGEKLELLTDKTENLMNEADRFQRTGRTLRRKMWWQNCKMKIVVALAVILLAVVIFLLVCFSGGNCLK

SEQ ID NO: 66 - DNA, 339 bp, Chlamydomonas reinhardtii
gcgactcctcttgtggccggattgagtgtggcggcggcagctttcgtgggcaagcaggtggtgcagacctacatcaagtttaagacttctccaggcctgttcaactccgtggggcggcaatactacaagggcggcttcctgcctgagatgacgcggcgagaggcggcgctgatcctgggcatccgggagtctgcgggcgaggagcgcgtcaaggacgcccaccggcgaatcatggttgcgaaccaccccgattctggcggcagcagctacgtcgccgccaaggtcaacgaagcaaaggacttgttgttggggaagaagaaggtcggaaagtcacccttc

SEQ ID NO: 67 - protein, 113 aa, Chlamydomonas reinhardtii
ATPLVAGLSVAAAAFVGKQVVQTYIKFKTSPGLFNSVGRQYYKGGFLPEMTRREAALILGIRESAGEERVKDAHRRIMVANHPDSGGSSYVAAKVNEAKDLLLGKKKVGKSPF

SEQ ID NO: 68 - DNA, 732 bp, Chlamydomonas reinhardtii
tcaccaacatatgctgagcatctgagcgtcatcaagggcttctgcgacaaggcagatggaaaggacaagcttaccgcgctcatacagtatgcctgcctcttcatttccgccggcgagcccggcaacatcaagaagatccaagcgtctgtgaccgccgcccggaaggtcttccgtgtcatgcggccactggagctcatgacgccgctgctgatcacgccgggcttcaccggcaagcagcccatgttgctggaggccgtcaacaaggtcaaggcggtgctcatggccgtgtacttcggtgccgatcacgtggtgtgggcgcaccagatcggcctcatcacagacaagaaggtcggcgagaggtaccagaagctgtcgctgtggtcttgggcgctgggcagcgtgtgcacggttgccacggagagctaccagattgcggccaagagcgtggtgcgcaaggagggcgagagcgaggaggactacgcgcggcgggtggaggaagttgaagaagcagatcaaccagcacctgtttgtgctcatccacggactgttccaggcagctctggcggcgggcctgctgcagctgctgccgctcaagccgcgcacggtgggcctgctgggcgtggtggcgtcggccatgaactgctacctgctgctgcccgcctaccccaagcccgtggccgctgccaaggccaagtcggcgtagaggcgtcgttaggcggccgtgcggcgttg

SEQ ID NO: 69 - protein, 244 aa, Chlamydomonas reinhardtii
SPTYAEHLSVIKGFCDKADGKDKLTALIQYACLFISAGEPGNIKKIQASVTAARKVFRVMRPLELMTPLLITPGFTGKQPMLLEAVNKVKAVLMAVYFGADHVVWAHQIGLITDKKVGERYQKLSLWSWALGSVCTVATESYQIAAKSVVRKEGESEEDYARRVEEVEEADQPAPVCAHPRTVPGSSGGGPAAAAAAQAAHGGPAGRGGVGHELLPAAARLPQARGRCQGQVGVEASLGGRAAL

SEQ ID NO: 70 - DNA, 429 bp, Chlamydomonas reinhardtii
cagagcagtcgctccatcggtgtggccagcacgtctggccgcgggagcagcagcagcagcctagcggccccccaacgcgtgcggacagggcgagctcgtgtggcccgcgtccaggcgttcgtggaccctgactccagtctggcgaccatccagaccgtctcccaggtcgttactacctgctgcattgcagccggcgctttcatgctcctgagcaagccaggggaatcgctcccagagcaggatcgcctggataaccgcaactcgcaaccgtgcccagtctgtggcggctcaggcttcgaggagtgcctctgctcgcgctggtcagatggcgacgtcgggtgcaactcgtgcagcaagacgggctacatgcggtgccgcggctgcgggggtggcggcacggctgtgcccctgatggtccgcgctcgcaag

SEQ ID NO: 71 - protein, 143 aa, Chlamydomonas reinhardtii
QSSRSIGVASTSGRGSSSSSLAAPQRVRTGRARVARVQAFVDPDSSLATIQTVSQVVTTCCIAAGAFMLLSKPGESLPEQDRLDNRNSQPCPVCGGSGFEECLCSRWSDGDVGCNSCSKTGYMRCRGCGGGGTAVPLMVRARK

SEQ ID NO: 72 - DNA, 645 bp, Chlamydomonas reinhardtii
gccctgaccatgcgttcctctagccttgttaaggccagccgcggccgcctgactgtgtgcgcggctacccccgtgcccaagcagttcaaggccgtgaagcccgttggcgatcgcgtgcttgtcaaggttgacaaggaggaggcgaagagcgttggcggtgtgctgctgccggcgtccgtgcgcaacaagcccacggctggctcgattattgctctgggcgatgccaagagtgtcaaggtaggaggcacacacgtacacgcacacacgtacacacgcgctcacgccgactgctccacccgcgcgcacgtcctgcaggaggaggacgtgattggtgtgctgtccgccagcgagaagattgcgcagctcaagcccctgtccgaccgcatcctcatcaagggcgccaaggccgaggacaagacctcgggcggtgtgctgctggcgactgagtcggcggagaagcccaccttcggcaccgtggtggctgtgggcgagggccgcgaggacgaggagaccaaggcgctggtcaagcccaacgtgaccgtgggcgccaccgtcatgtactccaagtactcggggacagagttcgaggaggacggcgacaactacattgtggtgcgcgagagcgacatcctggcccagctctcg

SEQ ID NO: 73 - protein, 215 aa, Chlamydomonas reinhardtii
ALTMRSSSLVKASRGRLTVCAATPVPKQFKAVKPVGDRVLVKVDKEEAKSVGGVLLPASVRNKPTAGSIIALGDAKSVKVGGTHVHAHTYTRAHADCSTRAHVLQEEDVIGVLSASEKIAQLKPLSDRILIKGAKAEDKTSGGVLLATESAEKPTFGTVVAVGEGREDEETKALVKPNVTVGATVMYSKYSGTEFEEDGDNYIVVRESDILAQLS

SEQ ID NO: 74 - DNA, 522 bp, Chlamydomonas reinhardtii
cagtcccttgcatcgcgcccctgcgtcctccgcgcgcagcgcccgttcaccgcgaagcgcagctcggtcgtccgcgtgcgtgccgaggcttcctcctcgtcgcctgtgcccatccccgtctggcctaaggacacagagagcgctcgcgatgtgtttgctttcggcggcagcgcgcccgagcgtgtgaacggccgcgtggccatgatgggcttcgtttccatcctggggcccgagctgtccaagaagcagccagtgctggagcaggtcggcgacgcctggtttggcatcctgctattctccctcaccatcaccttcgcctccatcctgcccaagctggtgtcgggtgtctccctgaaggagctgcactccgtggccacctctgagaacctgaagggcgagggcctgcagcaggcgctggcgctcttcgacaccaacgtggagctgtgggccggccgcctcgccatgctgggcttcgcgggcctgatcgccctggagaccattaagggcggcgaggctttcttc

SEQ ID NO: 75 - protein, 174 aa, Chlamydomonas reinhardtii
QSLASRPCVLRAQRPFTAKRSSVVRVRAEASSSSPVPIPVWPKDTESARDVFAFGGSAPERVNGRVAMMGFVSILGPELSKKQPVLEQVGDAWFGILLFSLTITFASILPKLVSGVSLKELHSVATSENLKGEGLQQALALFDTNVELWAGRLAMLGFAGLIALETIKGGEAFF

SEQ ID NO: 76 - DNA, 708 bp, Scenedesmus dimorphus
gcagccctgagccaggatgagctcaagcagcaggcagcctggaaggccgtggagtacgtgaagagcggcatggtggtcggccttggcacgggcagcacagctgccttcgcagttgaccgcatcggccagctgatgaaggagggcaccctcaaggacatcattggtgtgccaacctccatccgcacctatgagcaggcaaagtccctgggcatccctctggccactctggacgagcagccaaagattgatgtcgcaattgacggagcagatgaggttgacccaaagctggacgtggtcaagggccgtggtggcgcgctgctgcgcgagaagatggtggagatggcatctgccaagtttgtgtgcatcgtggatgacactaagctcgtggagggcctgggcggcagcaagctggccatgcctgtggagatcgtgcagttctgccacaagtacacactcaacaggcttgctgcgctgccggaggttgctggctgccaggccaagctgcgcatggagggcgacaagccctacgtgaccgacaacagcaactacatcgtggacctgtacttcgagacgcccatcaaggacagcgctgctgccagcaaggccatcctggcgctggagggcgtggtggaccacggcctgttcctggacatggtggacgtgtgcatcatcgcaggcaagaatggcgtggaggtgcaggagcgcaag

SEQ ID NO: 77 - protein, 236 aa, Scenedesmus dimorphus
AALSQDELKQQAAWKAVEYVKSGMVVGLGTGSTAAFAVDRIGQLMKEGTLKDIIGVPTSIRTYEQAKSLGIPLATLDEQPKIDVAIDGADEVDPKLDVVKGRGGALLREKMVEMASAKFVCIVDDTKLVEGLGGSKLAMPVEIVQFCHKYTLNRLAALPEVAGCQAKLRMEGDKPYVTDNSNYIVDLYFETPIKDSAAASKAILALEGVVDHGLFLDMVDVCIIAGKNGVEVQERK

(19) United States
(12) Patent Application Publication
(10) Pub. No.: US 2015/0089690 A1
(43) Pub. Date: Mar. 26, 2015

(54) SODIUM HYPOCHLORITE RESISTANT GENES

(71) Applicant: SAPPHIRE ENERGY, INC, San Diego, CA (US)

(72) Inventors: Christopher Yohn, San Diego, CA (US); Eric N. Hampton, San Diego, CA (US)

(73) Assignee: SAPPHIRE ENERGY, INC, San Diego, CA (US)

(21) Appl. No.: 14/378,747

(22) PCT Filed: Feb. 14, 2013
(86) PCT No.: PCT/US2013/026186

§ 371 Date: Aug. 14, 2014

Related U.S. Application Data
(60) Provisional application No. 61/598,736, filed on Feb. 14, 2012.

(51) Int. Cl. C12N 15/82; C07K 14/405

(52) U.S. Cl. CPC C12N 15/8274; C07K 14/405
USPC 800/300.1; 435/257.2; 800/300

(57) ABSTRACT

The present disclosure provides novel genes, identified from a Chlamydomonas reinhardtii cDNA library that when over expressed in algae confer sodium hypochlorite resistance to the organism. The present disclosure also provides methods of using the novel genes, and organism transformed by the novel genes.

### CROSS REFERENCE TO RELATED APPLICATIONS

This application claims the benefit of U.S. Provisional Patent Application Ser. No. 61/598,736, filed Feb. 14, 2012, of which is herein incorporated by reference in its entirety for all

### BACKGROUND

Algae are highly adaptable plants that are capable of rapid growth under a wide range of conditions. As photosynthetic organisms, they have the capacity to transform sunlight into energy that can be used to synthesize a variety of biomolecules for use as industrial enzymes, therapeutic compounds and proteins, nutritional, commercial, or fuel products, etc.

The majority of algal species are adapted to growth in an aqueous environment, and arc easily grown in liquid media using light as an energy source. The ability to grow algae on a large scale in an outdoor setting, in ponds or other open containers, using sunlight for photosynthesis, enhances their utility for bioproduction, environmental remediation, and carbon fixation.

When growing algae outdoors there exists a need to prevent microbes, predators (for example, chytrids or viruses), or competing algae strains from killing, overtaking, or slowing down the growth of a desired algae strain.

Sodium hypochlorite (bleach) has been used for decades as a household disinfectant. In addition, sodium hypochlorite is also commonly used in laboratories as an antimicrobial agent to sterilize contaminated cultures. The toxicity of sodium hypochlorite has been attributed to its ability to oxidize proteins and react with free amines, which causes protein unfolding, aggregation, and overall cellular chaos (Winter, J., et al., Cell (2008) 135(4):691-701).

Given sodium hypochlorite's mode of action, and its potential to act as a broad-spectrum biocide, it would be useful to modify an algae strain so that it is resistant to sodium hypochlorite.

The present disclosure provides novel genes, identified from a Chlamydomonas reinhardtii cDNA library that when over expressed in C. reinhardtii confer sodium hypochlorite resistance to the organism.

### SUMMARY

Described herein are novel genes, identified from a Chlamydomonas reinhardtii cDNA library that when over expressed in algae confer sodium hypochlorite resistance to the organism. The present disclosure also provides methods of using the novel genes, and organisms transformed by the novel genes.

Provided herein is a modified non-vascular photosynthetic organism transformed with a polynucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at least 90%, at least 95%, at least 98%, or at east 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; wherein expression of the protein encoded by the nucleic acid sequence or nucleotide sequence results in the transformed organism being tolerant to sodium hypochlorite at a concentration of from 0.01 ppm to 1 ppm, from 1 ppm to 5 ppm, from 5 ppm to 10 ppm, from 10 ppm to 15 ppm, from 15 ppm to 20 ppm, from 20 ppm to 25 ppm, from 25 ppm to 30 ppm, from 30 ppm to 35 ppm, from 35 ppm to 40 ppm, from 40 ppm to 45 ppm, from 45 ppm to 50 ppm, from 50 ppm to 55 ppm, from 55 ppm to 60 ppm, from 60 ppm to 65 ppm, from 65 ppm to 70 ppm, from 70 ppm to 75 ppm, from 75 ppm to 80 ppm, from 80 ppm to 85 ppm, from 85 ppm to 90 ppm, from 90 ppm to 9 ppm, from 95 ppm to ppm, from 100 ppm to 200 ppm, from 200 ppm to 500 ppm, from 500 ppm to 1000 ppm, from 1000 ppm to 2000 ppm, from 2000 ppm to 5000 ppm, from 5000 ppm to 10000 ppm, or from 10000 ppm to 15000 ppm. In one embodiment, the organism is grown in an aqueous environment. In other embodiments, the organism is an alga or a bacterium. In one embodiment, the alga is a microalga. In another embodiment, the bacterium is a cyanobacterium. In yet other embodiments, the microalga is at least one of a Chlamydomonas sp., Volvacales sp., Desmid sp., Dunaliella sp., Scenedesmus sp., Chlorella sp., Henatococcus sp., Volvox sp., Nannochloropsis sp., Arthrospira sp., Sprirulina sp., Botryococcus sp., Haematococcus sp., or Desmodesmus sp. In yet other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In other embodiments, the protein comprises an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Also provided herein is a modified non-vascular photosynthetic organism transformed with a nucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; wherein expression of the protein encoded by the nucleic acid sequence or nucleotide sequence results in the transformed organism having increased growth as compared to either an untransformed organism or a second transformed organism. In some embodiments, growth is measured by carrying capacity, culture productivity, or growth rate. In other embodiments, the growth of the transformed organism is from 0.01% to 1% greater than the untransformed organism or the second transformed organism, the growth of the transformed organism is from 1% to 10% greater than the untransformed organism or the second transformed organism, the growth of the transformed organism is from 10% to 20% greater than the untransformed organism or the second transformed organism, the growth of the transformed organism is from 20% to 40% greater than the untransformed organism or the second transformed organism, the growth of the transformed organism is from 40% to 60% greater than the untransformed organism or the second transformed organism, the growth of the transformed organism is from 60% to 80% greater than the untransformed organism or the second transformed organism, the growth of the transformed organism is from 80% to 100% greater than the untransformed organism or the second transformed organism, the growth of the transformed organism is from 100% to 200% greater than the untransformed organism or the second transformed organism, the growth of the transformed organism is from 200% to 300% greater than the untransformed organism or the second transformed organism, or the growth of the transformed organism is from 300% to 400% greater than the untransformed organism or the second transformed organism. In one embodiment, the organism is grown in an aqueous environment. In other embodiments, the concentration of sodium hypochlorite in the aqueous environment is from 1 ppm to 5,000 ppm, or the concentration of sodium hypochlorite in the aqueous environment is from 2 ppm to 60 ppm. In one embodiment, the organism is an alga or a bacterium. In another embodiment, the ala is a microalga. In one embodiment, the bacterium is a cyanobacterium. In other embodiments, the microalga is at least one of a Chlamydomonas sp., Volvacales sp., Desmid sp., Dunaliella sp., Scenedesmus sp., Chlorella sp., Haematococcus sp., Volvox sp., Nannochloropsis sp., Arthrospira sp., Sprirulina sp., Botryococcus sp., Haematococcus sp., or Desmodesmus sp. In yet other embodiments, the microalga is at least one of Chlamydomonas reinhardtii, N. oceanica, N. salina Dunaliella salina, H. pluvalis, S. dimorphus, Dunaliella viridis, N. oculata, Dunaliella tertiolecta, S. Maximus, or A. Fusiformus. In some embodiments, the protein comprises an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Further provided herein is a method of creating a non-vascular photosynthetic organism that is tolerant to sodium hypochlorite at a concentration of from 0.01 ppm to 1 ppm, from 1 ppm to 5 ppm, from 5 ppm to 10 ppm, from 10 ppm to 15 ppm, from 15 ppm to 20 ppm, from 20 ppm to 25 ppm, from 25 ppm to 30 ppm, from 30 ppm to 35 ppm, from 35 ppm to 40 ppm, from 40 ppm to 45 ppm, from 45 ppm to 50 ppm, from 50 ppm to 55 ppm, from 55 ppm to 60 ppm, from 60 ppm to 65 ppm, from 65 ppm to 70 ppm, from 70 ppm to 75 ppm, from 75 ppm to 80 ppm, from 80 ppm to 85 ppm, from 85 ppm to 90 ppm, from 90 ppm to 95 ppm, from 95 ppm to 100 ppm, from 100 ppm to 200 ppm, from 200 ppm to 500 ppm, from 500 ppm to 1000 ppm, from 1000 ppm to 2000 ppm, from 2000 ppm to 5000 ppm, from 5000 ppm to 10000 ppm, or from 10000 ppm to 15000 ppm, comprising: a) transforming the organism with a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74. In one embodiment, the transformed organism is grown in an aqueous environment. In one embodiment, the organism is an alga or a bacterium. In yet another embodiment, the alga is a microalga. In another embodiment, the bacterium is a cyanobacterium. In some embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In yet other embodiments, the microalga is at least one of Chlamydomonas reinhardtii, N. oceanica, N salina, Dunaliella salina, H. pluvalis, S. dimorphus, Dunaliella viridis, N. oculata, Dunaliella tertiolecta, S. Maximus, or A. Fusiformus. In some embodiments, the protein comprises an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, or 75.

Also provided herein is a modified higher plant transformed with a polynucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; wherein expression of the protein encoded by the nucleic acid sequence or nucleotide sequence results in the transformed higher plant being tolerant to sodium hypochlorite at a concentration of from 0.01 ppm to 1 ppm, from 1 ppm to 5 ppm, from 5 ppm to 10 ppm, from 10 ppm to 15 ppm, from 15 ppm to 20 ppm, from 20 ppm to 25 ppm, from 25 ppm to 30 ppm, from 30 ppm to 35 ppm, from 35 ppm to 40 ppm, from 40 ppm to 45 ppm, from 45 ppm to 50 ppm, from 50 ppm to 55 ppm, from 55 ppm to 60 ppm, from 60 ppm to 65 ppm, from 65 ppm to 70 ppm, from 70 ppm to 75 ppm, from 75 ppm to 80 ppm, from 80 ppm to 85 ppm, from 85 ppm to 90 ppm, from 90 ppm to 95 ppm, from 95 ppm to 100 ppm, from 100 ppm to 200 ppm, from 200 ppm to 500 ppm, from 500 ppm to 1000 ppm, from 1000 ppm to 2000 ppm, from 2000 ppm to 5000 ppm, from 5000 ppm to 10000 ppm, or from 10000 ppm to 15000 ppm. In some embodiments, the transformed higher plant is grown in an aqueous environment. In another embodiment, the transformed higher plant is Arabidopsis thaliana. In yet other embodiments, the transformed higher plant is a Brassica, Glycine, Gossypium, Medicago, Zea, Sorghum, Oryza, Triticum, or Panicum species. In some embodiments, the protein comprises an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Provided herein is a modified higher plant transformed with a nucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; wherein expression of the protein encoded by the nucleic acid sequence or nucleotide sequence results in the transformed higher plant having increased growth as compared to either an untransformed higher plant or a second transformed higher plant. In some embodiments, growth is measured by carrying capacity, culture productivity, or growth rate. In other embodiments, the growth of the transformed higher plant is from 0.01% to 1% greater than the untransformed higher plant or the second transformed higher plant, the growth of the transformed higher plant is from 1% to 10% greater than the untransformed higher plant or the second transformed higher plant, the growth of the transformed higher plant is from 10% to 20% greater than the untransformed higher plant or the second transformed higher plant, the growth of the transformed higher plant is from 20% to 40% greater than the untransformed higher plant or the second transformed higher plant, the growth of the transformed higher plant is from 40% to 60% greater than the untransformed higher plant or the second transformed higher plant, the growth of the transformed higher plant is from 60% to 80% greater than the untransformed higher plant or the second transformed higher plant, the growth of the transformed higher plant is from 80% to 100% greater than the untransformed higher plant or the second transformed higher plant, the growth of the transformed higher plant is from 100% to 200% greater than the untransformed higher plant or the second transformed higher plant, the growth of the transformed higher plant is from 200% to 300% greater than the untransformed higher plant or the second transformed higher plant, or the growth of the transformed higher plant is from 300% to 400% greater than the untransformed higher plant or the second transformed higher plant. In one embodiment, the transformed higher plant is grown in an aqueous environment. In other embodiments, the concentration of sodium hypochlorite in the aqueous environment is from 1 ppm to 5,000 ppm, or the concentration of sodium hypochlorite in the aqueous environment is from 2 ppm to 60 ppm. In one embodiment, the transformed higher plant is Arabidopsis thaliana. In other embodiments, the transformed higher plant is a Brassica, Glycine, Gossypium, Medicago, Zea, Sorghum, Oryza, Triticum, or Panicum species. In yet other embodiments, the protein compises an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Provided herein is a modified non-vascular photosynthetic organism transformed with a polynucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74: wherein expression of the protein encoded by the nucleic acid sequence or nucleotide sequence results in the transformed organism being tolerant to sodium hypochlorite at a concentration of about 1, of about 2, of about 2.5, of about 5, of about 10, of about 15, of about 20, of about 25, of about 30, of about 35, of about 40, of about 45, of about 50, of about 55, of about 60, of about 65, of about 70, of about 75, of about 80, of about 85, of about 90, of about 95, of about 100, of about 200, of about 500, of about 1,000, of about 2,000, or of about 5,000 ppm, or at a concentration of at least 2, of at least 2.5, of at least 5, of at least 10, of at least 15, of at least 20, of at least 25, of at least 30, of at least 35, of at least 40, of at least 45, of at least 50, of at least 55, of at least 60, of at least 65, of at least 70, of at least 75, of at least 80, of at least 85, of at least 90, of at least 95, of at least 100, of at least 200, of at least 500, of at least 1,000, of at least 2,000, or of at least 5,000 ppm. In one embodiment, the transformed organism is grown in an aqueous environment. In some embodiments, the transformed organism may be an alga or a bacterium. In one embodiment, the alga is a microalga. In another embodiment, the bacterium is a cyanobacterium. In other embodiments, the microalga is at least one of a Chlamydomonas sp., Volvacales sp., Dunaliella sp. Scenedesmus sp., Chlorella sp. Hematococcus sp., Volvox sp., Nannochloropsis sp., Arthrospira sp., Sprirulina sp., Botryococcus sp., Haematococcus sp., or Desmodesmus sp. In yet other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In some embodiments, the transformed organism may express a protein comprising an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Also provided herein is a modified non-vascular photosynthetic organism transformed with a nucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at last 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; wherein expression of the protein encoded by the nucleic acid sequence or nucleotide sequence results in the transformed organism having an increased growth as compared to either an untransformed organism or a second transformed organism. In some embodiments, the growth of the transformed organism may be at least 5% greater, at least 10% greater, at least 20% greater, at least 40% greater, at least 60% greater, at least 80% greater, at least 100% greater, at least 200% greater, at least 300% greater, or at least 400% greater than the untransformed organism or the second transformed organism. In some embodiments, an increase in growth may be measured by carrying capacity, culture productivity, or growth rate. In one embodiment, the transformed organism is grown in an aqueous environment. In other embodiments, the concentration of sodium hypochlorite in the aqueous environment is between 1 and 5,000 ppm. In yet other embodiments, the concentration of sodium hypochlorite in the aqueous environment is between 2 and 60 ppm. In some embodiments, the transformed organism may be an alga or a bacterium. In one embodiment, the alga is a microalga. In another embodiment, the bacterium is a cyanobacterium. In other embodiments, the microalga is at least one of a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In yet other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In some embodiments, the transformed organism may express a protein comprising an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Also provided is a method of creating a non-vascular photosynthetic organism that is tolerant to sodium hypochlorite at a concentration of about 1, of about 2, of about 2.5, of about 5, of about 10, of about 15, of about 20, of about 25, of about 30, of about 35, of about 40, of about 45, of about 50, of about 55, of about 60, of about 65, of about 70, of about 75, of about 80, of about 85, of about 90, of about 95, of about 100, of about 200, of about 500, of about 1,000, of about 2,000, or of about 5,000 ppm, or at a concentration of at least 2, of at least 2.5, of at least 5, of at least 10, of at least 15, of at least 20, of at least 25, of at least 30, of at least 35, of at least 40, of at least 45, of at least 50, of at least 55, of at least 60, of at least 65, of at least 70, of at least 75, of at least 80, of at least 85, of at least 90, of at least 95, of at least 100, of at least 200, of at least 500, of at least 1,000, of at least 2,000, or of at least 5,000 ppm, comprising: a) transforming the organism with a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74. In one embodiment, the transformed organism is grown in an aqueous environment. In some embodiments, the organism is an alga or a bacterium. In yet another embodiment, the alga is a microalga. In another embodiment, the bacterium is a cyanobacterium. In some embodiments, the microalga is at least one of a Chlamydomonas sp Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Hematococcus sp., or Desmodesmus sp. In yet other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In some embodiments, the transformed organism may express a protein comprising an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Also provided herein is a modified higher plant transformed with a polynucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; wherein expression of the protein encoded by the nucleic acid sequence or nucleotide sequence results in the transformed higher plant being tolerant to sodium hypochlorite at a concentration of about 1, of about 2, of about 2.5, of about 5, of about 10, of about 15, of about 20, of about 25, of about 30, of about 35, of about 40, of about 45, of about 50, of about 55, of about 60, of about 65, of about 70, of about 75, of about 80, of about 85, of about 90, of about 95, of about 100, of about 200, of about 500, of about 1,000, of about 2,000, or of about 5,000 ppm, or at a concentration of at least 2, of at least 2.5, of at least 5, of at least 10, of at least 15, of at least 20, of at least 25, of at least 30, of at least 35, of at least 40, of at least 45, of at least 50, of at least 55, of at least 60, of at least 65, of at least 70, of at least 75, of at least 80, of at least 85, of at least 90, of at least 95, of at least 100, of at least 200, of at least 500, of at least 1,000, of at least 2,000, or of at least 5,000 ppm. In one embodiment, the higher plant is grown in an aqueous environment. In one embodiment, the higher plant is Arabidopsis thaliana. In other embodiments, the higher plant is a Brassica, Glycine, Gossypium, Medicago, Zea, Sorghum, Oryza, Triticum, or Panicum species. In yet other embodiments, the protein comprises an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

Provided herein is a modified higher plant transformed with a nucleotide sequence comprising: a) a nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or b) a nucleotide sequence with at least 80%, at least 85%, at least 90%, at least 95%, at least 98%, or at least 99% sequence identity to the nucleic acid sequence of SEQ ID NO: 62, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; wherein expression of the protein encoded by the nucleic acid sequence or nucleotide sequence results in the transformed higher plant having increased growth as compared to either an untransformed higher plant or a second transformed higher plant. In some embodiments, the growth of the transformed higher plant is at least 5% greater, at least 10% greater, at least 20% greater, at least 40% greater, at least 60% greater, at least 80% greater, at least 100% greater, at least 200% greater, at least 300% greater, or at least 400% greater than the untransformed higher plant or the second transformed higher plant. In some embodiments, growth is measured by carrying capacity, culture productivity, or growth rate. In one embodiment, the higher plant is grown in an aqueous environment. In some embodiments, the concentration of sodium hypochlorite in the aqueous environment is between 1 and 5,000 ppm. In other embodiments, the concentration of sodium hypochlorite in the aqueous environment is between 2 and 60 ppm. In one embodiment, the higher plant is Arabidopsis thaliana. In other embodiments, the higher plant is a Brassica, Glycine, Gossypium, Medicago, Zea, Sorghum, Oryza, Triticum, or Panicum species. In yet other embodiments, the protein comprises an amino acid sequence of SEQ ID NO: 63, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77.

### BRIEF DESCRIPTION OF THE DRAWINGS

These and other features, aspects, and advantages of the present disclosure will become better understood with regard to the following description, appended claims and accompanying figures where:

For all 96-well plates described below and shown in the figures, the concentration of sodium hypochlorite across the top of the plate is as follows: 0, 0, 10, 10, 15, 15, 25, 25, 35, 35, 60, and 60 ppm; and the OD₇₅₀ of the cells was from bottom to top, 0.35, 0.325, 0.3, 0.25, 0.2, 0.15, 0.125, and 0.1.

FIG. 1 shows a representative 96-well plate of one candidate that passed (BF22) and one candidate that failed (BF25) a sodium hypochlorite microtiter validation assay.

FIG. 2 shows a 96-well plate containing wild-type Chlamydomonas reinhardtii, used as a comparison with the 96-well plates of FIG. 1.

FIG. 3 shows the results of a sodium hypochlorite radial diffusion assay. The y axis represents the area of sodium hypochlorite inhibition in cm². The x axis represents the different strains that were tested.

FIG. 4 shows several representative plates from the sodium hypochlorite radial diffusion assay. WT is wild type Chlamydomonas reinhardtii.

FIG. 5 shows the results of a sodium hypochlorite radial diffusion assay. The y axis represents the area of bleach inhibition in cm² and the x axis represents from left to right: 1 (WT): 2 (BF02); 3 (BF03); 4 (BF04); 5 (BF08); 6 (BF10/26); 7 (BF19); 8 (BF20); 9 (BF21); 10 (BF22); 11 (BF27); 12 (BF29); 13 (BF 30); 14 (BF32); 15 (BT06); 16 (BT07); 17 (BT08); AND 18 (BT11).

FIG. 6 is a cDNA overexpression vector (SENuc1060).

FIG. 7 is an image of a 96-well microtiter plate at the end of a sodium hypochlorite microtiter validation assay, for a strain transformed with BF2.

FIG. 8 is an image of a 96-well microtiter plate at the end of a sodium hypochlorite microtiter validation assay, for a strain transformed with BF3.

FIG. 9 is an image of a 96-well microtiter plate at the cud of a sodium hypochlorite microtiter validation assay, for a strain transformed with BF4.

FIG. 10 is an image of a 96-well microtiter plate at the end of a sodium hypochlorite microtiter validation assay, for a strain transformed with BF8.

FIG. 11 is an image of a 96-well microtiter plate at the end of a sodium hypochlorite microtiter validation assay, for a strain transformed with BF19.

FIG. 12 is an image of a 96-well microtiter plate at the end of a sodium hypochlorite microtiter validation assay, for a strain transformed with BF20.

FIG. 13 is an image of a 96-well microtiter plate at the end of a sodium hypochlorite microtiter validation assay, for a strain transformed with BF21.

FIG. 14 is an image of a 96-well microtiter plate at the end of a sodium hypochlorite microtiter validation assay, for a strain transformed with BF22.

FIG. 15 is an image of a 96-well microtiter plate at the end of a sodium hypochlorite microtiter validation assay, for a strain transformed with BF26.

FIG. 16 is an image of a 96-well microtiter plate at the end of a sodium hypochlorite microtiter validation assay, for a strain transformed with BF27.

FIG. 17 is an image of a 96-well microtiter plate at the end of a sodium hypochlorite microtiter validation assay, for a strain transformed with BF29.

FIG. 18 is an image of a 96-well microtiter plate at the end of a sodium hypochlorite microtiter validation assay, for a strain transformed with BF30.

FIG. 19 is an image of a 96-well microtiter plate at the end of a sodium hypochlorite microtiter validation assay, for a strain transformed with BT4.

FIG. 20 is an image of a 96-well microtiter plate at the end of a sodium hypochlorite microtiter validation assay, for a strain transformed with BT6.

FIG. 21 is an image of a 96-well microtiter plate at the end of a sodium hypochlorite microtiter validation assay, for a strain transformed with BT7.

FIG. 22 is an image of a 96-well microtiter plate at the end of a sodium hypochlorite microtiter validation assay, for a strain transformed with BT8.

FIG. 23 is an image of a 96-well microtiter plate at the end of a sodium hypochlorite microtiter validation assay, for a strain transformed with BT10.

FIG. 24 is an image of a 96-well microtiter plate at the end of a sodium hypochlorite microtiter validation assay, for wild type Chlamydomonas reinhardtii.

FIG. 25 is an image of plates of a sodium hypochlorite radial diffusion assay for a strain transformed with BF2. The upper left plate is the untransformed wild type strain.

FIG. 26 is an image of plates of a sodium hypochlorite radial diffusion assay for a strain transformed with BF3. The upper left plate is the untransformed wild type strain.

FIG. 27 is an image of plates of a sodium hypochlorite radial diffusion assay for a strain transformed with BF4. The upper left plate is the untransformed wild type strain.

FIG. 28 is an image of plates of a sodium hypochlorite radial diffusion assay for a strain transformed with BF8. The upper left plate is the untransformed wild type strain.

FIG. 29 is an image of plates of a sodium hypochlorite radial diffusion assay for a strain transformed with BF19. The upper left plate is the untransformed wild type strain.

FIG. 30 is an image of plates of a sodium hypochlorite radial diffusion assay for a strain transformed with BF20. The upper left plate is the untransformed wild type strain.

FIG. 31 is an image of plates of a sodium hypochlorite radial diffusion assay for a strain transformed with BF21. The upper left plate is the untransformed wild type strain.

FIG. 32 is an image of plates of a sodium hypochlorite radial diffusion assay for a strain transformed with BF22. The upper left plate is the untransformed wild type strain.

FIG. 33 is an image of plates of a sodium hypochlorite radial diffusion assay for a strain transformed with BF26. The upper left plate is the untransformed wild type strain.

FIG. 34 is an image of plates of a sodium hypochlorite radial diffusion assay for a strain transformed with BF27. The upper left plate is the untransformed wild type strain.

FIG. 35 is an image of plates of a sodium hypochlorite radial diffusion may for a strain transformed with BF29. The upper left plate is the untransformed wild type strain.

FIG. 36 is an image of plates of a sodium hypochlorite radial diffusion assay for a strain transformed with BF30. The upper left plate is the untransformed wild type strain.

FIG. 37 is an image of plates of a sodium hypochlorite radial diffusion assay for a strain transformed with BF32. The upper left plate is the untransformed wild type strain.

FIG. 38 is an image of plates of a sodium hypochlorite radial diffusion assay for a strain transformed with BT4. The upper left plate is the untransformed wild type strain.

FIG. 39 is an image of plates of a sodium hypochlorite radial diffusion assay for a strain transformed with BT6. The upper left plate is the untransformed wild type strain.

FIG. 40 is an image of plates of a sodium hypochlorite radial diffusion assay for a strain transformed with BT7. The upper left plate is the untransformed wild type strain.

FIG. 41 is an image of plates of a sodium hypochlorite radial diffusion assay for a strain transformed with BT8. The upper left plate is the untransformed wild type strain.

FIG. 42 is an image of plates of a sodium hypochlorite radial diffusion assay for a strain transformed with BT10. The upper left plate is the untransformed wild type strain.

FIG. 43 is an image of plates of a sodium hypochlorite radial diffusion assay for a strain transformed with BT11. The upper left plate is the untransformed wild type strain.

FIG. 44 is an image of plates of a sodium hypochlorite radial diffusion assay for the untransformed wild type C. reinhardtii strain.

FIG. 45 is an image of plates of a sodium hypochlorite radial diffusion assay for the untransformed wild type C. reinhardtii strain.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present inventive discovery.

As used in this specification and the appended claims, the singular forms “a”, “an” and “the” include plural reference unless the context clearly dictates otherwise.

Sodium Hypochlorite Resistance

The sodium hypochlorite resistant organism (for example, alga) is grown in media containing a concentration of sodium hypochlorite that permits growth of the transformed organism, but inhibits growth of the same species of organism that is not transformed, to confer resistance to sodium hypochlorite. The concentration for optimal production of a product by the host organism and/or inhibition of growth of other nontransformed species can be empirically determined.

The terms “tolerant” and “resistant” can be used interchangeably throughout the specification, as well as any variation of the two words such as “tolerance” and “resistance.”

Endogenous

An endogenous nucleic acid, nucleotide, polypeptide, or protein as described herein is defined in relationship to the host organism. An endogenous nucleic acid, nucleotide, polypeptide, or protein is one that naturally occurs in the host organism.

Exogenous

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

Nucleic Acid and Protein Sequences

The following nucleic acid and amino acid sequences are useful in the disclosed embodiments.

If an initial start codon (Met) is not present in any of the amino acid sequences disclosed herein, including sequences contained in the sequence listing, one of skill in the art would be able to include, at the nucleotide level, an initial ATG, so that the translated polypeptide would have the initial Met. If a start and/or stop codon is not present at the beginning and/or cod of a coding sequence, one of skill in the art would know to insert an “ATG” at the beginning of the coding sequence and nucleotides encoding for a stop codon (any one of TAA, TAG, or TGA) at the end of the coding sequence. Several of the nucleotide sequences disclosed herein are missing an initial “ATG” and/or are missing a stop codon. Any of the disclosed nucleotide sequences can be, if desired, fused to another nucleotide sequence that when operably linked to a “control element” results in the proper translation of the encoded amino acids (for example, a fusion protein). In addition, two or more nucleotide sequences can be linked by a short peptide, for example, a viral peptide.

SEQ ID NO: 1 is a primer. “V” is any one of A, C or G. “N” is any one of A, T, C or G.

SEQ ID NO: 2 is a primer.

SEQ ID NO: 3 is a primer.

SEQ ID NO: 4 is a primer.

SEQ ID NO: 5 is a primer.

SEQ ID NO: 6 is a primer.

SEQ ID NO: 7 is a primer.

SEQ ID NO: 8 is the nucleic acid sequence of BF02.

SEQ ID NO: 9 is the nucleic acid sequence of BF03.

SEQ ID NO: 10 is the nucleic acid sequence of BF04.

SEQ ID NO: 11 is the nucleic acid sequence of BF08.

SEQ ID NO: 12 is the nucleic acid sequence of BF10/BF26.

SEQ ID NO: 13 is the nucleic acid sequence of BF19.

SEQ ID NO: 14 is the nucleic acid sequence of BF20.

SEQ ID NO: 15 is the nucleic acid sequence of BF21.

SEQ ID NO: 16 is the nucleic acid sequence of BF22.

SEQ ID NO: 17 is the nucleic acid sequence of BF27.

SEQ ID NO: 18 is the nucleic acid sequence of BF29.

SEQ ID NO: 19 is the nucleic acid sequence of BF30.

SEQ ID NO: 20 is the nucleic acid sequence of BF32.

SEQ ID NO: 21 is the nucleic acid sequence of BT06.

SEQ ID NO: 22 is the nucleic acid sequence of BT07.

SEQ ID NO: 23 is the nucleic acid sequence of BT08.

SEQ ID NO: 24 is the nucleic acid sequence of BT11.

SEQ ID NO: 25 is the translated sequence of SEQ ID NO: 8.

SEQ ID NO: 26 is the translated sequence of SEQ ID NO: 9.

SEQ ID NO: 27 is the translated sequence of SEQ ID NO: 10.

SEQ ID NO: 28 is the translated sequence of SEQ ID NO: 11.

SEQ ID NO: 29 is the translated sequence of SEQ ID NO: 12.

SEQ ID NO: 30 is the translated sequence of SEQ ID NO: 13.

SEQ ID NO: 31 is the translated sequence of SEQ ID NO: 14.

SEQ ID NO: 32 is the translated sequence of SEQ ID NO: 15.

SEQ ID NO: 33 is the translated sequence of SEQ ID NO: 16.

SEQ ID NO: 34 is the translated sequence of SEQ ID NO: 17.

SEQ ID NO: 35 is the translated sequence of SEQ ID NO: 18.

SEQ ID NO: 36 is the translated sequence of SEQ ID NO: 19.

SEQ ID NO: 37 is the translated sequence of SEQ ID NO: 20.

SEQ ID NO: 38 is the translated sequence of SEQ ID NO: 21.

SEQ ID NO: 39 is the translated sequence of SEQ ID NO: 22.

SEQ ID NO: 40 is the translated sequence of SEQ ID NO: 23.

SEQ ID NO: 41 is the translated sequence of SEQ ID NO: 24.

SEQ ID NO: 42 is SEQ ID NO: 8 without a start codon and a stop codon.

SEQ ID NO: 43 is SEQ ID NO: 25 without an initial “M”.

SEQ ID NO: 44 is SEQ ID NO: 9 without a start codon and a stop codon.

SEQ ID NO: 45 is SEQ ID NO: 26 without an initial “M”.

SEQ ID NO: 46 is SEQ ID NO: 10 without a start codon and a stop codon.

SEQ ID NO: 47 is SEQ ID NO: 27 without an initial “M”.

SEQ ID NO: 48 is SEQ ID NO: 11 without a start codon and a stop codon.

SEQ ID NO: 49 is SEQ ID NO: 28 without an initial “M”.

SEQ ID NO: 50 is SEQ ID NO: 12 without a start codon and a stop codon.

SEQ ID NO: 51 is SEQ ID NO: 29 without an initial “M”.

SEQ ID NO: 52 is SEQ ID NO: 13 without a start codon and a stop codon.

SEQ ID NO: 53 is SEQ ID NO: 30 without an initial “M”.

SEQ ID NO: 54 is SEQ ID NO: 14 without a start codon and a stop codon.

SEQ ID NO: 55 is SEQ ID NO: 31 without an initial “M”.

SEQ ID NO: 56 is SEQ ID NO: 15 without a start codon and a stop codon.

SEQ ID NO: 57 is SEQ ID NO: 32 without an initial “M”.

SEQ ID NO: 58 is SEQ ID NO: 16 without a start codon and a stop codon.

SEQ ID NO: 59 is SEQ ID NO: 33 without an initial “M”.

SEQ ID NO: 60 is SEQ ID NO: 17 without a start codon and a stop codon.

SEQ ID NO: 61 is SEQ ID NO: 34 without an initial “M”.

SEQ ID NO: 62 is SEQ ID NO: 18 without a start codon and a stop codon.

SEQ ID NO: 63 is SEQ ID NO: 35 without an initial “M”.

SEQ ID NO: 64 is SEQ ID NO: 19 without a start codon and a stop codon.

SEQ ID NO: 65 is SEQ ID NO: 36 without an initial “M”.

SEQ ID NO: 66 is SEQ ID NO: 20 without a start codon and a stop codon.

SEQ ID NO: 67 is SEQ ID NO: 37 without an initial “M”.

SEQ ID NO: 68 is SEQ ID NO: 21 without a start codon and a stop codon.

SEQ ID NO: 69 is SEQ ID NO: 38 without an initial “M”.

SEQ ID NO: 70 is SEQ ID NO: 22 without a star codon and a stop codon.

SEQ ID NO: 71 is SEQ ID NO: 39 without an initial “M”.

SEQ ID NO: 72 is SEQ ID NO: 23 without a start codon and a stop codon.

SEQ ID NO: 73 is SEQ ID NO: 40 without an initial “M”.

SEQ ID NO: 74 is SEQ ID NO: 24 without a start codon and a stop codon.

SEQ ID NO: 75 is SEQ ID NO: 41 without an initial “M”.

SEQ ID NO: 76 is the nucleic acid sequence of a gene from Scenedesmus dimorophus with 85% sequence identity to SEQ ID NO: 62, SEQ ID NO: 76 does not have a start or a stop codon.

SEQ ID NO: 77 is the translated sequence of SEQ ID NO: 76.

Host Cells or Host Organisms

Biomass useful in the methods and systems described herein can be obtained from host cells or host organisms that have been modified (e.g. genetically engineered) to be, for example, sodium hypochlorite resistant, as compared to an unmodified organism. In addition, the host cells or host organism can be further modified to express an exogenous or endogenous protein, such as a protein involved in the isoprenoid biosynthetic pathway or a protein involved in the accumulation and/or secretion of fatty acids, glycerol lipids, or oils.

A host cell can contain a polynucleotide encoding a polypeptide of the present disclosure. In some embodiments, a host cell is part of a multicellular organism. In other embodiments, a host cell is cultured as a unicellular organism.

Host organisms can include any suitable host, for example, a microorganism. Microorganisms which are useful for the methods described herein include, for example, photosynthetic bacteria (e.g., cyanobacteria), non-photosynthetic bacteria (e.g., E. coli), yeast (e.g., Saccharomyces cerevisiae), and algae (e.g., microalgae such as Chlamydomonas reinhardtii).

Examples of host organisms that can be transformed with a polynucleotide of interest (for example, a polynucleotide that encodes a protein involved in the isoprenoid biosynthesis pathway) include vascular and non-vascular organisms. The organism can be prokaryotic or eukaryotic. The organism can be unicellular or multicellular. A host organism is an organism comprising a host cell. In other embodiments, the host organism is photosynthetic. A photosynthetic organism is one that naturally photosynthesizes (e.g., an alga) or that is genetically engineered or otherwise modified to be photosynthetic. In some instances, a photosynthetic organism may be transformed with a construct or vector of the disclosure which renders all or part of the photosynthetic apparatus inoperable.

By way of example, a non-vascular photosynthetic microalga species (for example, C. reinhardtii, Nannochloropsis oceania, N. salina, D. salina, H. pluvalis, S. dimorphus, D. viridis, Chlorella sp., and D. tertiolecta) can be genetically engineered to produce a polypeptide of interest, for example a fusicoccadiene synthase or an FPP synthase. Production of a fusicoccadiene synthase or an FPP synthase in these microalgae can be achieved by engineering the microalgae to express the fusicoccadiene synthase or FPP synthase in the algal chloroplast or nucleus.

In other embodiments the host organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays) coconut (Cocos nucifera), palm (Elaeis guineensis), oil nut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

The host cell can be prokaryotic. Examples of some prokaryotic organisms of the present disclosure include, but are not limited to, cyanobacteria (e.g., Synechococcus, Synechocystis, Athrospira, Anacytis, Anabaena, Nostoc, Spirulina, Fremyella, Gleocapsa, Oscillatoria, and, Pseudoanabaena). Suitable prokaryotic cells include, but are not limited to, any of a variety of laboratory strains of Escherichia coli, Lactobacillus sp., Salmonella sp., and Shigella sp. (for example, as described in Carrier et al. (1992) J. Immunol. 148:1176-1181; U.S. Pat. No. 6,447,784; and Sizemore et al. (1995) Science 270:299-302). Examples of Salmonella strains which can be employed in the present disclosure include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Pseudomonas pudita, Pseudomonas aeruginosa, Pseudomonas mevalonii, Rhodobacter sphaeroides, Rhodobacter capsulatus, Rhodospirillum rubrum, and Rhodococcus sp.

In some embodiments, the host organism is eukaryotic (e.g. green algae, red algae, brown algae). In some embodiments, the algae is a green algae, for example, a Chlorophycean. The algae can be unicellular or multicellular. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans. Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, and Chlamydomonas reinhardtii.

In some embodiments, eukaryotic microalgae, such as for example, a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp., can be used in the disclosed methods.

In other embodiments, the host cell is Chlamydomonas reinhardtii, Dunaliella salina, Haematococcus pluvialis, Nannochloropsis oceania, Nannochloropsis salina, Dunaliella salina, Scenadesmus dimorphus, a Chlorella species, a Spirulina species, a Desmid species, Spirulina maximus, Arthrospira fusiformis, Dunaliella viridis, Nannochloropsis oculata, or Dunaliella tertiolecta.

In some instances the organism is a rhodophyte, chlorophyte, heterokontophyte, tribophyte, glaucophyte, chlorarachniophyte, cuglenoid, haptophyte, cryptomonad, dinoflagellum, or phytoplankton.

In some instances a host organism is vascular and photosynthetic. Examples of vascular plants include, but are not limited to, angiosperms, gymnosperms, rhyniophytes, or other tracheophytes.

In some instances a host organism is non-vascular and photosynthetic. As used herein, the term “non-vascular photosynthetic organism,” refers to any macroscopic or microscopic organism, including, but not limited to, algae, cyanobacteria and photosynthetic bacteria, which does not have a vascular system such as that found in vascular plants. Examples of non-vascular photosynthetic organisms include bryophtyes, such as marchantiophytes or anthocerotophytes. In some instances the organism is a cyanobacteria. In some instances, the organism is algae (e.g., macroalgae or microalgae). The algae can be unicellular or multicellular algae. For example, the microalgae Chlamydomonas reinhardtii may be transformed with a vector, or a linearized portion thereof, encoding one or more proteins of interest (e.g., a protein involved in the isoprenoid biosynthesis pathway).

Methods for algal transformation are described in U.S. Provisional Patent Application No. 60/142,091. The methods of the present disclosure can be carried out using algae, for example, the microalga, C. reinhardtii. The use of microalgae to express a polypeptide or protein complex according to a method of the disclosure provides the advantage that large populations of the microalgae can be grown, including commercially (Cyanotech Corp.; Kailua-Kona Hi.), thus allowing for production and, if desired, isolation of large amounts of a desired product.

The vectors of the present disclosure may be capable of stable or transient transformation of multiple photosynthetic organisms, including, but not limited to, photosynthetic bacteria (including cyanobacteria), cyanophyta, prochlorophyta, rhodophyta, chlorophyta, pyrrophyta, heterokontophyta, tribophyta, glaucophyta, chlorarachniophytes, euglenophyta, euglenoids, haptophyta, chrysophyta (including diatoms) cryptophyta, cryptomonads, dinophyta, dinoflagellata, pyrmnesiophyta, bacillariophyta, xanthophyta, eustigmatophyta, raphidophyta, phaeophyta, and phytoplankton. Other vectors of the present disclosure are capable of stable or transient transformation of, for example, C. reinhardtii, N. oceania, N. salina, D. salina, H. pluvalis, S. dimorphus, D. viridis, or D. tertiolecta.

Examples of appropriate hosts, include but are not limited to: bacterial cells, such as E. coli, Streptomyces, Salmonella typhimurium; fungal cells, such as yeast; insect cells, such as Drosophila S2 and Spodoptera S9; animal cells, such as CHO, COS or Bowes melanoma; adenoviruses; and plant cells. The selection of an appropriate host is deemed to be within the scope of those skilled in the art.

Polynucleotides selected and isolated as described herein are introduced into a suitable host cell. A suitable host cell is any cell which is capable of promoting recombination and/or reductive reassortment. The selected polynucleotides can be, for example, in a vector which includes appropriate control sequences. The host cell can be, for example, a higher eukaryotic cell, such as a mammalian cell, or a lower eukaryotic cell, such as a yeast cell, or the host cell can be a prokaryotic cell, such as a bacterial cell. Introduction of a construct (vector) into the host cell can be effected by, for example, calcium phosphate transfection, DEAE-Dextran mediated transfection, or electroporation.

Recombinant polypeptides, including protein complexes, can be expressed in plants, allowing for the production of crops of such plants and, therefore, the ability to conveniently produce large amounts of a desired product. Accordingly, the methods of the disclosure can be practiced using any plant, including, for example, microalga and macroalgae, (such as marine algae and seaweeds), as well as plants that grow in soil.

In one embodiment, the host cell is a plant. The term “plant” is used broadly herein to refer to a eukaryotic organism containing plastids, such as chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, and roots. A part of a plant useful for propagation includes, for example, seeds, fruits, cuttings, seedlings, tubers, and rootstocks.

The genes of the present disclosure can be expressed in a higher plant. For example, Arabidopsis thaliana. The genes can also be expressed in a Brassica, Glycine, Gossypium, Medicago, Zea, Sorghum, Oryza, Triticum, or Panicum species.

A method of the disclosure can generate a plant containing genomic DNA (for example, a nuclear and/or plastid genomic DNA) that is genetically modified to contain a stably integrated polynucleotide (for example, as described in Hager and Bock, Appl. Microbial. Biotechnol. 54:302-310, 2000). Accordingly, the present disclosure further provides a transgenic plant, e.g. C. reinhardtii, which comprises one or more chloroplasts containing a polynucleotide encoding one or more exogenous or endogenous polypeptides, including polypeptides that can allow for secretion of fuel products and/or fuel product precursors (e.g., isoprenoids, fatty acids, lipids, triglycerides). A photosynthetic organism of the present disclosure comprises at least one host cell that is modified to generate, for example, a fuel product or a fuel product precursor.

Some of the host organisms useful in the disclosed embodiments are, for example, are extremophiles, such as hyperthermophiles, psychrophiles, psychrotrophs, halophiles, barophiles and acidophiles. Some of the host organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, a host cell expressing a protein of the present disclosure can be grown in a liquid environment which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 31, 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid environments.

Where a halophilic organism is utilized for the present disclosure, it may be transformed with any of the vectors described herein. For example, D. salina may be transformed with a vector which is capable of insertion into the chloroplast or nuclear genome and which contains nucleic acids which encode a protein (e.g., an FPP synthase or a fusicoccadiene synthase). Transformed halophilic organisms may then be grown in high-saline environments (e.g., salt lakes, salt ponds, and high-saline media) to produce the products (e.g., lipids) of interest. Isolation of the products may involve removing a transformed organism from a high-saline environment prior to extracting the product from the organism. In instances where the product is secreted into the surrounding environment, it may be necessary to desalinate the liquid environment prior to any further processing of the product.

The present disclosure further provides compositions comprising a genetically modified host cell. A composition comprises a genetically modified host cell; and win in some embodiments comprise one or more further components, which components are selected based in part on the intended use of the genetically modified host cell. Suitable components include, but are not limited to, salts; buffers; stabilizers; protease-inhibiting agents; cell membrane- and/or cell wall-preserving compounds, e.g., glycerol and dimethylsulfoxide; and nutritional media appropriate to the cell.

For the production of a protein, for example, an isoprenoid or isoprenoid precursor compound, a host cell can be, for example, one that produces, or has been genetically modified to produce, one or more enzymes in a prenyl transferase pathway and/or a mevalonate pathway and/or an isoprenoid biosynthetic pathway. In some embodiments, the host cell is one that produces a substrate of a prenyl transferase, isoprenoid synthase or mevalonate pathway enzyme.

In some embodiments, a genetically modified host cell is a host cell that comprises an endogenous mevalonate pathway and/or isoprenoid biosynthetic pathway and/or prenyl transferase pathway. In other embodiments, a genetically modified host cell is a host cell that does not normally produce mevalonate or IPP via a mevalonate pathway, or FPP, GPP or GGPP via a prenyl transferase pathway, but has been genetically modified with one or more polynucleotides comprising nucleotide sequences encoding one or more mevalonate pathway, isoprenoid synthase pathway or prenyl transferees pathway enzymes (for example, as described in U.S. Patent Publication No. 2004/005678; U.S. Patent Publication No. 2003/0148479; and Martin et al. (2003) Nat. Biotech. 21(7):796-802).

Culturing of Cell or Organisms

An organism may be grown under conditions which permit photosynthesis, however, this is not a requirement (e.g., a host organism may be grown in the absence of light). In some instances, the host organism may be genetically modified in such a way that its photosynthetic capability is diminished or destroyed. In growth conditions where a host organism is not capable of photosynthesis (e.g., because of the absence of light and/or genetic modification), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or an organism-specific requirement. Organic carbon sources include any source of carbon which the host organism is able to metabolize including, but not limited to, acetate, simple carbohydrates (e.g., glucose, sucrose, and lactose), complex carbohydrates (e.g., starch and glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

Optimal growth of organisms occurs usually at a temperature of about 20° C. to about 25° C., although some organisms can still grow at a temperature of up to about 35° C. Active growth is typically performed in liquid culture. If the organisms are grown in a liquid medium and are shaken or mixed, the density of the cells can be anywhere from about 1 to 5×10⁸ cells/ml at the stationary phase. For example, the density of the cells at the stationary phase for Chlamydomonas sp. can be about 1 to 5×10⁷ cells/ml; the density of the cells at the stationary phase for Nannochloropsis sp. can be about 1 to 5×10⁸ cells/ml; the density of the cells at the stationary phase for Scenedesmus sp. can be about 1 to 5×10⁷ cells/ml; and the density of the cells at the stationary phase for Chlorella sp. can be about 1 to 5×10⁸ cells/ml. Exemplary cell densities at the stationary phase are as follows: Chlamydomonas sp. can be about 1×10⁷ cells/ml: Nannochloropsis sp. can be about 1×10⁸ cells/ml; Scenedesmus sp. can be about 1×10⁷ cells/ml; and Chlorella sp. can be about 1×10⁸ cells/ml. An exemplary growth rate may yield, for example, a two to twenty fold increase in cells per day, depending on the growth conditions. In addition, doubling times for organisms can be, for example, 5 hours to 30 hours. The organism can also be grown on solid media, for example, media containing about 1.5% agar, in plates or in slants.

One source of energy is fluorescent light that can be placed, for example, at a distance of about 1 inch to about two feet from the organism. Examples of types of fluorescent lights includes, for example, cool white and daylight. Bubbling with air or CO₂ improves the growth rate of the organism. Bubbling with CO₂ can be, for example, at 1% to 5% CO₂. If the lights are turned on and off at regular intervals (for example, 12:12 or 14:10 hours of light:dark) the cells of some organisms will become synchronized.

Long term storage of organisms can be achieved by streaking them onto plates, sealing the plates with, for example, Parafilm™, and placing them in dim light at about 10° C. to about 18° C. Alternatively, organisms may be grown as streaks or stabs into agar tubes, capped, and stored at about 10° C. to about 18° C. Both methods allow for the storage of the organisms for several months.

For longer storage, the organisms can be grown in liquid culture to mid to late log phase and then supplemented with a penetrating cryoprotective agent like DMSO or MeOH, and stored at less than −130° C. An exemplary range of DMSO concentrations that can be used is 5 to 8%. An exemplary range of MeOH concentrations that can be used is 3 to 9%.

For longer Organisms can be grown on a defined minimal medium (for example, high salt medium (HSM), modified artificial sea water medium (MASM), or F/2 medium) with light as the sole energy source. In other instances, the organism can be grown in a medium (for example, tris acetate phosphate (TAP) medium), and supplemented with an organic carbon source. Organisms, such as algae, can grow naturally in fresh water or marine water. Culture media for freshwater algae can be, for example, synthetic media, enriched media, soil water media, and solidified media, such as agar. Various culture media have ban developed and used for the isolation and cultivation of fresh water algae and are described in Watanabe. M. W. (2005). Freshwater Culture Media. In R. A. Andersen (Ed.), Algal Culturing Techniques (pp. 13-20). Elsevier Academic Press. Culture media for marine algae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In some instances, organisms can be grown in containers wherein each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container.

Culturing techniques for algae are well known to one of skill in the an and are described, for example, in Freshwater Culture Media. In R. A. Andersen (Ed.), Algal Culturing Techniques. Elsevier Academic Press.

Because photosynthetic organisms, for example, algae, require sunlight. CO₂ and water for growth, they can be cultivated in, for example, open ponds and lakes. However, these open systems are more vulnerable to contamination than a closed system. One challenge with using an open system is that the organism of interest may not grow as quickly as a potential invader. This becomes a problem when another organism invades the liquid environment in which the organism of interest is growing, and the invading organism has a faster growth rate and takes over the system.

In addition, in open systems there is less control over water temperature, CO₂ concentration, and lighting conditions. The growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system are that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In these ponds, the organism, water, and nutrients circulate around a “racetrack” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors.

Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

The raceway ponds can be operated in a continuous manner, with, for example, CO₂ and nutrients being constantly fed to the ponds, while water containing the organism is removed at the other end.

If the raceway pond is placed outdoors, there are several different ways to address the invasion of an unwanted organism. For example, the pH or salinity of the liquid in which the desired organism is in can be such that the invading organism either slows down its growth or dies.

Also, chemicals can be added to the liquid, such as bleach, or a pesticide can be added to the liquid, such as glyphosate. In addition, the organism of interest can be genetically modified such that it is better suited to survive in the liquid environment. Any one or more of the above strategies can be used to address the invasion of an unwanted organism.

Alternatively, organisms, such as algae, can be grown in closed structures such as photobioreactors, where the environment is under stricter control than in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations.

On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethylene bag cultivation). A batch photobioreactor is set up with, for example, nutrients, an organism (for example, algae), and water, and the organism is allowed to grow until the batch is harvested. A continuous photobioreactor can be harvested, for example, either continually, daily, or at fixed time intervals.

High density photobioreactors are described in, for example, Lee, et al., Biotech. Bioengineering 44:1161-1167, 1994. Other types of bioreactors, such as those for sewage and waste water treatments, are described in, Sawayama, et al., Appl. Micro. Biotech., 41:729-731. 1994. Additional examples of photobioreactors are described in, U.S. Appl. Publ. No. 2005/0260553, U.S. Pat. No. 5,958,761, and U.S. Pat. No. 6,083,740. Also, organisms, such as algae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermentors. Additional methods of culturing organism and variations of the methods described herein are known to one of skill in the art.

Organisms can also be grown near ethanol production plants or other facilities or regions (e.g., cities and highways) generating CO₂. As such, the methods herein contemplate business methods for selling carbon credits to ethanol plants or other facilities or regions generating CO₂ while making fuels or fuel products by growing one or more of the organisms described herein near the ethanol production plant, facility, or region.

The organism of interest, grown in any of the systems described herein, can be, for example, continually harvested, or harvested one batch at a time.

CO₂ can be delivered to any of the systems described herein, for example, by bubbling in CO₂ from under the surface of the liquid containing the organism. Also, sparges can be used to inject CO₂ into the liquid. Spargers are, for example, porous disc or tube assemblies that are also referred to as Bubblers, Carbonators, Acrators, Porous Stones and Diffusers.

Nutrients that can be used in the systems described herein include, for example, nitrogen (in the form of NO₃⁻ or NH₄⁺), phosphorus, and trace metals (Fe, Mg, K, Ca, Co, Cu, Mn, Mo, Zn, V, and B). The nutrients can come, for example, in a solid form or in a liquid form. If the nutrients are in a solid form they can be mixed with, for example, fresh or salt water prior to being delivered to the liquid containing the organism, or prior to being delivered to a photobioreactor.

Organisms can be grown in cultures, for example large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at lease 5 square meters, at least 10 square meters, at least 200 square meters, at least 500 square meters, at least 1,500 square meters, at least 2,500 square meters, in area, or greater.

Chlamydomonas sp., Nannochloropsis sp., Scenedesmus sp., and Chlorella sp. are exemplary algae that can be cultured as described herein and can grow under a wide array of conditions.

One organism that can be cultured as described herein is a commonly used laboratory species C. reinhardtii. Cells of this species are haploid, and can grow on a simple medium of inorganic salts, using photosynthesis to provide energy. This organism can also grow in total darkness if acetate is provided as a carbo source. C. reinhardtii can be readily grown at room temperature under standard fluorescent lights. In addition, the cells can be synchronized by placing them on a light-dark cycle. Other methods of culturing C. reinhardtii cells are known to one of skill in the art.

Polynucleotides and Polypeptides

In addition to being genetically engineered to be, for example, sodium hypochlorite resistant, as compared to an unengineered organism, the host cells or host organism can be further modified to express an exogenous or endogenous protein, for example, a protein involved in the isoprenoid biosynthetic pathway or a protein involved in the accumulation and/or secretion of fatty acids, glycerol lipids, or oils.

Also provided are isolated polynucleotides encoding a protein, for example, an FPP synthase, described herein. As used herein “isolated polynucleotide” means a polynucleotide that is free of one or both of the nucleotide sequences which flank the polynucleotide in the naturally-occurring genome of the organism from which the polynucleotide is derived. The term includes, for example, a polynucleotide or fragment thereof that is incorporated into a vector or expression cassette; into an autonomously replicating plasmid or virus; into the genomic DNA of a prokaryote or eukaryote; or that exists as a separate molecule independent of other polynucleotides. It also includes a recombinant polynucleotide that is part of a hybrid polynucleotide, for example, one encoding a polypeptide sequence.

The proteins of the present disclosure can be made by any method known in the art. The protein may be synthesized using either solid-phase peptide synthesis or by classical solution peptide synthesis also known as liquid-phase peptide synthesis. Using Val-Pro-Pro, Enalapril and Lisinopril as starting templates, several series of peptide analogs such as X-Pro-Pro, X-Ala-Pro, and X-Lys-Pro, wherein X represents any amino acid residue, may be synthesized using solid-phase or liquid-phase peptide synthesis. Methods for carrying out liquid phase synthesis of libraries of peptides and oligonucleotides coupled to a soluble oligomeric support have also been described. Bayer, Ernst and Mutter, Manfred, Nature 237:512-513 (1972); Bayer, Ernst, et al., J. Am. Chem. Soc. 96:7333-7336 (1974); Bonora, Gian Maria, et al., Nucleic Acids Res. 18:3155-3159 (1990). Liquid phase synthetic methods have the advantage over solid phase synthetic methods in that liquid phase synthesis methods do not require a structure present on a first reactant which is suitable for attaching the reactant to the solid phase. Also, liquid phase synthesis methods do not require avoiding chemical conditions which may cleave the bond between the solid phase and the first reactant (or intermediate product). In addition, reactions in a homogeneous solution may give better yields and more complete reactions than those obtained in heterogeneous solid phase/liquid phase systems such as those present in solid phase synthesis.

In oligomer-supported liquid phase synthesis the growing product is attached to a large soluble polymeric group. The product from each step of the synthesis can then be separated from unreacted reactants based on the large difference in size between the relatively large polymer-attached product and the unreacted reactants. This permits reactions to take place in homogeneous solutions, and eliminates tedious purification steps associated with traditional liquid phase synthesis. Oligomer-supported liquid phase synthesis has also been adapted to automatic liquid phase synthesis of peptides. Bayer, Ernst, et al., Peptides: Chemistry, Structure, Biology, 426-432.

For solid-phase peptide synthesis, the procedure entails the sequential assembly of the appropriate amino acids into a peptide of a desired sequence while the end of the growing peptide is linked to an insoluble support. Usually, the carboxyl terminus of the peptide is linked to a polymer from which it can be liberated upon treatment with a cleavage reagent. In a common method, an amino acid is bound to a resin particle, and the peptide generated in a stepwise manner by successive additions of protected amino acids to produce a chain of amino acids. Modifications of the technique described by Merrifield are commonly used. See, e.g., Merrifield, J. Am. Chem. Soc. 96: 2989-93 (1964). In an automated solid-phase method, peptides are synthesized by loading the carboxy-terminal amino acid onto an organic linker (e.g., PAM, 4-oxymethylphenylacetamidomethyl), which is covalently attached to an insoluble polystyrene resin cross-linked with divinyl benzene. The terminal amine may be protected by blocking with t-butyloxycarbonyl. Hydroxyl- and carboxyl-groups are commonly protected by blocking with O-benzyl groups. Synthesis is accomplished in an automated peptide synthesizer, such as that available from Applied Biosystems (Foster City, Calif.). Following synthesis, the product may be removed from the resin. The blocking groups are removed by using hydrofluoric acid or trifluoromethyl sulfonic acid according to established methods. A routine synthesis may produce 0.5 mmole of peptide resin. Following cleavage and purification, a yield of approximately 60 to 70% is typically produced. Purification of the product peptides is accomplished by, for example, crystallizing the peptide from an organic solvent such as methyl-butyl ether, then dissolving in distilled water, and using dialysis (if the molecular weight of the subject peptide is greater than about 500 daltons) or reverse high pressure liquid chromatography (e.g., using a C¹⁸ column with 0.1% trifluoracetic acid and acetonitrile as solvents) if the molecular weight of the peptide is less than 500 daltons. Purified peptide may be lyophilized and stored in a dry state until use. Analysis of the resulting peptides may be accomplished using the common methods of analytical high pressure liquid chromatography (HPLC) and electrospray mass spectrometry (ES-MS).

In other cases, a protein, for example, a protein involved in the isoprenoid biosynthesis pathway or in fatty acid synthesis, is produced by recombinant methods. For production of any of the proteins described herein, host cells transformed with an expression vector containing the polynucleotide encoding such a protein can be used. The host cell can be a higher eukaryotic cell, such as a mammalian cell, or a lower eukaryotic cell such as a yeast or algal cell, or the host can be a prokaryotic cell such as a bacterial cell. Introduction of the expression vector into the host cell can be accomplished by a variety of methods including calcium phosphate transfection, DEAE-dextran mediated transfection, polybrene, protoplast fusion, liposomes, direct microinjection into the nuclei, scrape loading, biolistic transformation and electroporation. Large scale production of proteins from recombinant organisms is a well-established process practiced on a commercial scale and well within the capabilities of one skilled in the art.

It should be recognized that the present disclosure is not limited to transgenic cells, organisms, and plastids containing a protein or proteins as disclosed herein, but also encompasses such cells, organisms, and plastids transformed with additional nucleotide sequences encoding enzymes involved in fatty acid synthesis. Thus, some embodiments involve the introduction of one or more sequences encoding proteins involved in fatty acid synthesis in addition to a protein disclosed herein. For example, several enzymes in a fatty acid production pathway may be linked, either directly or indirectly, such that products produced by one enzyme in the pathway, once produced, are in close proximity to the next enzyme in the pathway. These additional sequences may be contained in a single vector either operatively linked to a single promoter or linked to multiple promoters, e.g. one promoter for each sequence. Alternatively, the additional coding sequences may be contained in a plurality of additional vectors. When a plurality of vectors are used, they can be introduced into the host cell or organism simultaneously or sequentially.

Additional embodiments provide a plastid, and in particular a chloroplast, transformed with a polynucleotide encoding a protein of the present disclosure. The protein may be introduced into the genome of the plastid using any of the methods described herein or otherwise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga at al., Methods in Plant Molecular Biology, Cold Spring Harbor Laboratory Press, 1995; Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding any one or more of the proteins of the present disclosure. Methods for the assembly of artificial plastid genomes can be found in co-pending U.S. patent application Ser. No. 12/287,230 filed Oct. 6, 2008, published as U.S. Publication No. 2009/0123977 on May 14, 2009, and U.S. patent application Ser. No. 12/384,893 filed Apr. 8, 2009, published as U.S. Publication No. 2009/0269816 on Oct. 29, 2009, each of which is incorporated by reference in its entirety.

One or more nucleotides of the present disclosure can also be modified such that the resulting amino acid is “substantially identical” to the unmodified or reference amino acid.

A “substantially identical” amino acid sequence is a sequence that differs from a reference sequence by one or more conservative or non-conservative amino acid substitutions, deletions, or insertions, particularly when such a substitution occurs at a site that is not the active site (catalytic domains (CDs)) of the molecule and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine).

The disclosure provides alternative embodiments of the polypeptides of the invention (and the nucleic acids that encode them) comprising at least one conservative amino acid substitution, as discussed herein (e.g., conservative amino acid substitutions are those that substitute a given amino acid in a polypeptide by another amino acid of like characteristics). The invention provides polypeptides (and the nucleic acids that encode them) wherein any, some or all amino acids residues are substituted by another amino acid of like characteristics, e.g., a conservative amino acid substitution.

Conservative substitutions are those that substitute a given amino acid in a polypeptide by another amino acid of like characteristics. Examples of conservative substitutions are the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. In alternative aspects, these conservative substitutions can also be synthetic equivalents of these amino acids.

Introduction of Polynucleotide into a Host Organism or Cell

To generate a genetically modified boast cell, a polynucleotide, or a polynucleotide cloned into a vector, is introduced stably or transiently into a host cell, using established techniques, including, but not limited to, electroporation, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance.

A polynucleotide or recombinant nucleic acid molecule described herein, can be introduced into a cell (e.g., alga cell) using any method known in the art. A polynucleotide can be introduced into a cell by a variety of methods, which are well known in the art and selected, in part, based on the particular host cell. For example, the polynucleotide can be introduced into a cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann. Rev. Plant. Physiol. Plant Mol. Biol. 42:205-225, 1991).

As discussed above, microprojectile mediated transformation can be used to introduce a polynucleotide into a cell (for example, as described in Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a cell using a device such as the BIOLISTIC PD-1000 particle gun (BioRad; Hercules Calif.). Methods for the transformation using biolistic methods are well known in the art (for example, as described in Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494498, 1996; and Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, and the glass bead agitation method.

The basic techniques used for transformation and expression in photosynthetic microorganisms are similar to those commonly used for E. coli, Saccharomyces cerevisiae and other species. Transformation methods customized for a photosynthetic microorganisms, e.g., the chloroplast of a strain of algae, ar known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (See, for example, Sanford, Trends In Biotech. (1988) 6: 299-302, U.S. Pat. No. 4,945,050; electroporation (Fromm et al., Proc. Nat'l. Acad. Sci. (USA) (1985) 82: 5824-5828); use of a laser beam, electroporation, microinjectin or any other method capable of introducing DNA into a host cell.

Plastid transformation is a routine and well known method for introducing a polynucleotide into a plant cell chloroplast (sec U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

A further refinement in chloroplast transformation/expression technology that facilitates control over the timing and tissue pattern of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the protein can be modified for plastid targeting by employing plant cell nuclear transformation constructs wherein DNA coding sequences of interest are fused to any of the available transit peptide sequences capable of facilitating transport of the encoded enzymes into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of DNAs encoding the enzymes. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, or LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heijne et al. (1991) Plant Mol. Biol. Rep. 9: 104; Clark et al. (1989) J. Biol. Chem. 264: 17544; della-Cioppa et al. (1987) Plant Physiol. 84: 965; Romer et al. (1993) Biochem. Biophys. Res. Commun. 196: 1414; and Shah et al. (1986) Science 233: 478. Another transit peptide sequence is that of the intact ACCase from Chlamydomonas (genbank EDO96563, amino acids 1-33). The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present disclosure are not critical as long as delivery into a plastid is obtained. Proteolytic processing within the plastid then produces the mature enzyme. This technique has proven successful with enzymes involved in polyhydroxyalkanoate biosynthesis (Nawrath et al. (1994) Proc. Natl. Acad Sci. USA 91: 12760), and neomycin phosphotransferase II (NPT-II) and CP4 EPSPS (Padgette at al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, α-carboxy-transferase, and plastid-targeted monocot multifunctional acetyl-CoA carboxylase (Mw, 220,000); plastidic subunits of the fatty acid synthase complex (e.g., acyl carrier protein (ACP), malonyl-ACP synthase, KASI, KASII, and KASIII); steroyl-ACP desaturase; thioesterases (specific for short, medium, and long chain acyl ACP); plastid-targeted acyl transferases (e.g., glycerol-3-phosphate and acyl transferase); enzymes involved in the biosynthesis of aspartate family amino acids: phytoene synthase; gibberllic acid biosynthesis (e.g., ent-kaurene synthases 1 and 2); and carotenoid biosynthesis (e.g., lycopene synthase).

Nuclear transformation of eukaryotic algal cells can be by microprojectile mediated transformation, or can be by protoplast transformation, electroporation, introduction of DNA using glass fibers, or the glass bead agitation method, as nonlimiting examples (Kindle, Proc. Natl. Acad. Sciences USA 87: 1228-1232 (1990); Shimogawara et al. Genetics 148: 1821-1828 (1998)). Markers for nuclear transformation of algae include, without limitation, markers for rescuing auxotrophic strains (e.g., NIT1 and ARG7 in Chlamydomonas; Kindle et al. J. Cell Biol. 109: 2589-2601 (1989), Debuchy et al. EMBO J. 8: 2803-2809 (1989)), as well as dominant selectable markers (e.g., CRY1, aada; Nelson et al. Mol. Cellular Biol. 14: 4011-4019 (1994), Cerutti et al. Genetics 145: 97-110 (1997)). In some embodiments, the presence of the knock out is used as a selectable marker for transformants. A knock out sequence can in some embodiments be co-transformed with a second sequence encoding a protein to be produced by the alga (for example, a therapeutic protein, industrial enzyme) or a protein that promotes or enhances production of a commercial, therapeutic, or nutritional product. The second sequence is in some embodiments provided on the same nucleic acid construct as the knock out sequence for transformation into the alga, in which the success of the knock out sequence in activating the gene of interest is used as the selectable marker.

In some embodiments, an alga is transformed with a nucleic acid which encodes a protein of interest, for example, a prenyl transferase, an isoprenoid synthase, or an enzyme capable of converting a precursor into a fuel product or a precursor of a fuel product (e.g., an isoprenoid or fatty acid).

In one embodiment, a transformation may introduce a nucleic acid into a plastid of the host alga (e.g., chloroplast). In another embodiments a transformation may introduce a nucleic acid into the nuclear genome of the host alga. In still another embodiment, a transformation may introduce nucleic acids into both the nuclear genome and into a plastid.

Transformed cells can be plated on selective media following introduction of exogenous nucleic acids. This method may also comprise several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which (which chelates magnesium) is added to chelate toxic metals. Following the screening for clones with the proper integration of exogenous nucleic acids, clones can be screened for the presence of the encoded protein(s) and/or products. Protein expression screening can be performed by Western blot analysis and/or enzyme activity assays. Transporter and/or product screening may be performed by any method known in the art, for example ATP turnover assay, substrate transport assay, HPLC or gas chromatography.

The expression of the protein or enzyme can be accomplished by inserting a polynucleotide sequence (gene) encoding the protein or enzyme into the chloroplast or nuclear genome of a microalgae. The modified strain of microalgae can be made homoplasmic to ensure that the polynucleotide will be stably maintained in the chloroplast genome of all descendents. A microalga is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein. The process of determining the plasmic state of an organism of the present disclosure involves screening transformants for the presence of exogenous nucleic acids and the absence of wild-type nucleic acids at a given locus of interest.

Vectors

Construct, vector and plasmid are used interchangeably throughout the disclosure. Nucleic acids encoding the proteins described herein, can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences.

In some embodiments, a polynucleotide of the present disclosure is cloned or inserted into an expression vector using cloning techniques know to one of skill in the art. The nucleotide sequences may be inserted into a vector by a variety of methods. In the most common method the sequences are inserted into an appropriate restriction endonuclease site(s) using procedures commonly known to those skilled in the art and detailed in, for example, Sambrook at al., Molecular Cloning. A Laboratory Manual, 2nd Ed., Cold Spring Harbor Press, (1989) and Ausubel et al., Short Protocols in Molecular Biology, 2nd Ed., John Wiley & Sons (1992).

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, and herpes simplex virus), P1-based artificial chromosomes, yeast plasmids, yeast artificial chromosomes, and any other vectors specific for specific hosts of interest (such as E. coli and yeast). Thus, for example, a polynucleotide encoding an FPP synthase, can be inserted into any one of a variety of expression vectors that are capable of expressing the enzyme. Such vectors can include, for example, chromosomal, nonchromosomal and synthetic DNA sequences.

Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used. For example, vectors such as Ble2A, Arg7/2A, and SEnuc357 can be used for the expression of a protein.

Numerous suitable expression vectors are known to those of skill in the art. The following vectors are provided by way of example; for bacterial host cells: pQE vectors (Qiagen), pBluescript plasmids, pNH vectors, lambda-ZAP vectors (Stratagene), pTrc99a, pKK223-3, pDR540, and pRIT2T (Pharmacia); for eukaryotic host cells: pXT1, pSG5 (Stratagene), pSVK3, pBPV, pMSG, pET21a-d(+) vectors (Novagen), and pSVLSV40 (Pharmacia). However, any other plasmid or other vector may be used so long as it is compatible with the host cell.

The expression vector, or a linearized portion thereof, can encode one or more exogenous or endogenous nucleotide sequences. Examples of exogenous nucleotide sequences that can be transformed into a host include genes from bacteria, fungi, plants, photosynthetic bacteria or other algae. Examples of other types of nucleotide sequences that can be transformed into a host, include, but are not limited to, transporter genes, isoprenoid producing genes, genes which encode for proteins which produce isoprenoids with two phosphates (e.g., GPP synthase and/or FPP synthase), genes which encode for proteins which produce fatty acids, lipids, or triglycerides, for example, ACCases, endogenous promoters, and 5′ UTRs from the psbA, atpA, or rbcL genes. In some instances, an exogenous sequence is flanked by two homologous sequences.

Homologous sequences are, for example, those that have at least 50%, at least 60%, at least 70%, at least 80%, at least 90%, at least 95%, at least 98%, or at least at least 99% sequence identity to a reference amino acid sequence or nucleotide sequence, for example, the amino acid sequence or nucleotide sequence that is found naturally in the host cell. The first and second homologous sequences enable recombination of the exogenous or endogenous sequence into the genome of the host organism. The first and second homologous sequences can be at least 100, at least 200, at least 300, at least 400, at least 500, or at least 1500 nucleotides in length.

The polynucleotide sequence may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased or chloroplast codon biased). In some embodiments, codon biasing occurs before mutagenesis to generate a polypeptide. In other embodiments, codon biasing occurs after mutagenesis to generate a polynucleotide. In yet other embodiments, codon biasing occurs before mutagenesis as well as after mutagenesis. Codon bias is described in detail herein.

In some embodiments, a vector comprises a polynucleotide operably linked to one or more control elements, such as a promoter and/or a transcription terminator. A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook t al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

A vector in some embodiments provides for amplification of the copy number of a polynucleotide. A vector can be, for example, an expression vector that provides for expression of an ACCase, a prenyl transferase, an isoprenoid synthase, or a mevalonate synthesis enzyme in a host cell, e.g., a prokaryotic host cell or a eukaryotic host cell.

A polynucleotide or polynucleotides can be contained in a vector or vectors. For example, where a second (or more) nucleic acid molecule is desired, the second nucleic acid molecule can be contained in a vector, which can, but need not be, the same vector as that containing the first nucleic acid molecule. The vector can be any vector useful for introducing a polynucleotide into a genome and can include a nucleotide sequence of genomic DNA (e.g., nuclear or plastid) that is sufficient to undergo homologous recombination with genomic DNA, for example, a nucleotide sequence comprising about 400 to about 1500 or more substantially contiguous nucleotides of genomic DNA.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the cytosol, nucleus, chloroplast membrane or cell membrane) can be attached to the polynucleotide encoding a protein or interest. Such signals are well known in the at and have been widely reported (see, e.g., U.S. Pat. No. 5,776,689).

Promoters are untranslated sequences located generally 100 to 1000 base pairs (bp) upstream from the start codon of a structural gene that regulate the transcription and translation of nucleic acid sequences under their control.

Promoters useful for the present disclosure may come from any source (e.g., viral, bacterial, fungal, protist, and animal). The promoters contemplated herein can be specific to photosynthetic organisms, non-vascular photosynthetic organisms, and vascular photosynthetic organisms (e.g., algae, flowering plants). In some instances, the nucleic acids above are inserted into a vector that comprises a promoter of a photosynthetic organism, e.g., algae. The promoter can be a constitutive promoter or an inducible promoter. A promoter typically includes necessary nucleic acid sequences near the start site of transcription, (e.g., a TATA element). Common promoters used in expression vectors include, but are not limited to, LTR or SV40 promoter, the E. coli lac or trp promoters, and the phage lambda PL promoter. Other promoters known to control the expression of genes in prokaryotic or eukaryotic cells can be used and we known to those skilled in the art. Expression vectors may also contain a ribosome binding site for translation initiation, and a transcription terminator. The vector may also contain sequences useful for the amplification of gene expression.

A “constitutive” promoter is a promoter that is active under most environmental and developmental conditions. An “inducible” promoter is a promoter that is active under controllable environmental or developmental conditions. Examples of inducible promoters/regulatory elements include, for example, a nitrate-inducible promoter (for example, as described in Bock et al, Plant Mol. Biol. 17:9 (1991)), or a light-inducible promoter, (for example, as described in Feinbaum et al, Mol Gen. Genet. 226:449 (1991); and Lam and Chua, Science 248:471 (1990)), or a heat responsive promoter (for example, as described in Muller et al., Gene 111: 165-73 (1992)).

In many embodiments, a polynucleotide of the present disclosure includes a nucleotide sequence encoding a protein or enzyme of the present disclosure, where the nucleotide sequence encoding the polypeptide is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (for example, as described in Guzman ea al. (1995) J. Bacteriol. 177:41214130); a xylose-inducible promoter, e.g., Pxyl (for example, as described in Kim et al. (1996) Gene 181:71-76); a GAL1 promoter a tryptophan promoter, a lac promoter; an alcohol-inducible promoter, e.g., a methanol-inducible promoter, an ethanol-inducible promoter, a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

In many embodiments a polynucleotide of the present disclosure includes a nucleotide sequence encoding a protein or enzyme of the present disclosure, where the nucleotide sequence encoding the polypeptide is operably linked to a constitutive promoter. Suitable constitutive promoters for use in prokaryotic cells are known in the art and include, but are not limited to, a sigma70 promoter, and a consensus sigma70 promoter.

Suitable promoters for use in prokaryotic host cells include, but are not limited to, a bacteriophage T7 RNA polymerase promoter; a trp promoter; a lac operon promoter, a hybrid promoter, e.g., a lac/tac hybrid promoter, a tac/trc hybrid promoter, a trp/lac promoter, a T7/lac promoter; a Ire promoter, a tac promoter; an araBAD promoter; in vivo regulated promoters, such as an ssaG promoter or a related promoter (for example, as described in U.S. Patent Publication No. 20040131637), a pagC promoter (for example, as described in Pulkkinen and Miller, J. Bacteriol., 1991: 173(1): 86-93; and Alpuche-Aranda et al., PNAS, 1992; 89(21): 10079-83), a nirB promoter (for example, as described in Harborne et al. (1992) Mol. Micro. 6:2805-2813; Dunstan et al. (1999) Infect. Immun. 67:5133-5141; McKelvie t al. (2004) Vaccine 22:3243-3255; and Chatfield et al. (1992) Biotechnol. 10:888-892); a sigma70 promoter, e.g., a consensus sigma70 promoter (for example, GenBank Accession Nos. AX798980, AX798961, and AX798183); a stationary phase promoter, e.g., a dps promoter, an spy promoter, a promoter derived from the pathogenicity island SPI-2 (for example, as described in WO96/17951); an actA promoter (for example, as described in Shetron-Rama et al. (2002) Infect. Immun. 70:1087-1096); an rpsM promoter (for example, as described in Valdivia and Falkow (1996). Mol. Microbiol. 22:367-378); a tet promoter (for example, as described in Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); and an SP6 promoter (for example, as described in Melton at al. (1984) Nucl. Acids Res. 12:7035-7056).

In yeast, a number of vectors containing constitutive or inducible promoters may be used. For a review of such vectors see, Current Protocols in Molecular Biology, Vol. 2, 1988, Ed. Ausubel, at al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover. 1986, DNA Cloning, Vol. II, IRL Press, Wash., D.C., Ch. 3; Bitter, 1987, Heterologous Gene Expression in Yeast, Methods in Enzymology, Eds. Berger & Kimmel, Acad. Press. N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (for example, as described in Cloning in Yeast, Ch. 3, RI Rothstein In: DNA Cloning Vol. 11, A Practical Approach, Ed. DM Glover, 1986, IRL Press, Wash., D.C.). Alternatively, vectors may be used which promote integration of foreign DNA sequences into the yeast chromosome.

Non-limiting examples of suitable eukaryotic promoter include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain, for example, one or more cloning sites such as a multiple cloning site, which can, but need not, be positioned such that a exogenous or endogenous polynucleotide can be inserted into the vector and operatively linked to a desired element.

The vector also can contain a prokaryote origin of replication (ori), for example, an E. coli ori or a cosmid ori, thus allowing passage of the vector into a prokaryote host cell, as well as into a plant chloroplast. Various bacterial and viral origins of replication are well known to those skilled in the art and include, but are not limited to the pBR322 plasmid origin, the 2u plasmid origin, and the SV40, polyoma, adenovirus, VSV, and BPV viral origins.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, an IRES. Additionally, an element can be a cell compartmentalization signal (i.e., a sequence that targets a polypeptide to the cytosol, nucleus, chloroplast membrane or cell membrane). In some aspects of the present disclosure, a cell compartmentalization signal (e.g., a cell membrane targeting sequence) may be ligated to a gene and/or transcript, such that translation of the gene occurs in the chloroplast. In other aspects, a cell compartmentalization signal may be ligated to a gene such that, following translation of the gene, the protein is transported to the cell membrane. Cell compartmentalization signals are well known in the art and have been widely reported (see, e.g., U.S. Pat. No. 5,776,689).

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (for example, as described in Giacomin, Plant Sci. 116:59-72, 1996; Scikantha, J. Bacteriol. 178:121, 1996; Gerdes, FEBS Lett. 389:44-47, 1996; and Jefferson, EMBO J. 6:3901-3907, 1997, fl-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain, for example, prokaryotic cells, eukaryotic cells, and/or plant cells that express the marker and, therefore, can be useful as a component of a vector of the disclosure. The selection gene or marker can encode for a protein necessary for the survival or growth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin and paromycin (for example, as described in Herrera-Estrella, EMBO J. 2.987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of bistidine (for example, as described in Hartman, Proc. Natl. Acad. Sci., USA 85:8047, 1988); mannose-6-phosphate isomerase which allows cells to utilize mannose (for example, as described in PC Publication Application No. WO 94/20627); omithine decarboxylase, which confers resistance to the ornithine decarboxylase inhibitor, 2-(difluoromethyl)-DL-omithine (DFMO; for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee at al., EMBO J. 7:1241-1248, 1988), a mutant psbA, which confers resistance to atrazine (for example, as described in Smeda at al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, dtreptomycin, streptomycin, sulfonamide and sulfonylurea resistance in plants (for example, as described in Maliga et al., Methods in Plant Molecular Biology, Cold Spring Harbor Laboratory Press, 1995, page 39). The selection marker can have its own promoter or its expression can be driven by a promoter driving the expression of a polypeptide of interest.

Reporter genes greatly enhance the ability to monitor gene expression in a number of biological organisms. Reporter genes have been successfully used in chloroplasts of higher plants, and high levels of recombinant protein expression have been reported. In addition, reporter genes have been used in the chloroplast of C. reinhardtii. In chloroplasts of higher plants, 3-glucuronidase (uidA, for example, as described in Staub and Malign, EMBO J. 12:601-606, 1993), neomycin phosphotransferase (nptII, for example, as described in Carrer et al., Mol. Gen. Genet. 241:49-56, 1993), adenosyl-3-adenyltransf-erase (aadA, for example, as described in Svab and Maliga, Proc. Natl. Acad Sci., USA 90:913-917, 1993), and the Aequorea victoria GFP (for example, as described in Sidorov et al., Plant J. 19:209-216, 1999) have been used as reporter genes (for example, as described in Heifetz, Biochemie 82:655-666, 2000). Each of these genes has attributes that make them useful reporters of chloroplast gene expression, such as ease of analysis, sensitivity, or the ability to examine expression in situ. Based upon these studies, other exogenous proteins have been expressed in the chloroplasts of higher plants such as Bacillus thuringiensis Cry toxins, conferring resistance to insect herbivores (for example, as described in Kota t al., Proc. Natl. Acad. Sci., USA 96:1840-1845, 1999), or human somatotropin (for example, as described in Staub t al., Nat. Biotechnol. 18:333.338, 2000), a potential biopharmaceutical. Several reporter genes have been expressed in the chloroplast of the eukaryotic green alga, C. reinhardtii, including aadA (for example, as described in Goldschmidt-Clermont, Nucl Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 1993; and Ishikura et al., J. Biosci Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet 263:404-410, 2000). In one embodiment the protein described herein is modified by the addition of an N-terminal strep tag epitope to add in the detection of protein expression.

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh at al., Genetics 152:1111-1122, 1999, each of which is incorporated herein by reference). The entire chloroplast genome of C. reinhardtii is available to the public on the world wide web, at the URL “biology.duke.edu/chlamy_genome/-chloru.html” (see “view complete genome as text file” link and “maps of the chloroplast genome” link; J. Maul, J. W. Lilly, and D. B. Stern, unpublished results; revised Jan. 28, 2002; to be published as GenBank Ace. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maul, J. E., et al. (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlamy/chloro/chloro140.html”).

In addition, the entire nuclear genome of C. reinhardtii is described in Merchant, S. S., et al., Science (2007), 318(5848):245-250, thus facilitating one of skill in the art to select a sequence or sequences useful for constructing a vector.

For expression of the polypeptide in a host, an expression cassette or vector may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to the gene, or may be derived from an exogenous source. Expression vectors generally have convenient restriction sites located near the promoter sequence to provide for the insertion of nucleic acid sequences encoding exogenous or endogenous proteins. A selectable marker operative in the expression host may be present.

The nucleotide sequences may be inserted into a vector by a variety of methods. In the most common method the sequences are inserted into an appropriate restriction endonuclease site(s) using procedures commonly known to those skilled in the at and detailed in, for example, Sambrook at al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

The description herein provides that host cells may be transformed with vectors. One of skill in the art will recognize that such transformation includes transformation with circular or linearized vectors, or linearized portions of a vector. Thus, a host cell comprising a vector may contain the entire vector in the cell (in either circular or liner form), or may contain a linearized portion of a vector of the present disclosure. In some instances 0.5 to 1.5 kb flanking nucleotide sequences of chloroplast genomic DNA may be used. In some instances 0.5 to 1.5 kb flanking nucleotide sequences of nuclear genomic DNA may be used, or 2.0 to 5.0 kb may be used.

Compounds

The modified or transformed host organism disclosed herein is useful in the production of a desired biomolecule, compound, composition, or product; these terms can be used interchangeably. The present disclosure provides methods of producing, for example, an isoprenoid or isoprenoid precursor compound in a host cell. One such method involves, culturing a modified host cell in a suitable culture medium under conditions that promote synthesis of a product, for example, an isoprenoid compound or isoprenoid precursor compound, where the isoprenoid compound is generated by the expression of an enzyme of the present disclosure, wherein the enzyme uses a substrate present in the host cell. In some embodiments, a method further comprises isolating the isoprenoid compound from the cell and/or from the culture medium.

In some embodiments, the product (e.g. fuel molecule) is collected by harvesting the liquid medium. As some fuel molecules (e.g., monoterpenes) are immiscible in water, they would float to the surface of the liquid medium and could be extracted easily, for example by skimming. In other instances, the fuel molecules can be extracted from the liquid medium. In still other instances, the fuel molecules are volatile. In such instances, impermeable barriers can cover or otherwise surround the growth environment and can be extracted from the air within the barrier. For some fuel molecules, the product may be extracted from both the environment (e.g., liquid environment and/or air) and from the intact host cells. Typically, the organism would be harvested at an appropriate point and the product may then be extracted from the organism. The collection of cells may be by any means known in the art, including, but not limited to concentrating cells, mechanical or chemical disruption of cells, and purification of product(s) from cell cultures and/or cell lysates. Cells and/or organisms can be grown and then the product(s) collected by any means known to one of skill in the an. One method of extracting the product is by harvesting the host cell or a group of host cells and then drying the cell(s). The product(s) from the dried host cell(s) are then harvested by crushing the cells to expose the product. In some instances, the product may be produced without killing the organisms. Producing and/or expressing the product may not render the organism unviable.

In some embodiments, a genetically modified host cell is cultured in a suitable medium (e.g., Luria-Bertoni broth, optionally supplemented with one or more additional agents, such as an inducer (e.g., where the isoprenoid synthase is under the control of an inducible promoter); and the culture medium is overlaid with an organic solvent e.g. dodecane, forming an organic layer. The compound produced by the genetically modified host partitions into the organic layer, from which it can then be purified. In some embodiments, where, for example, a prenyl transferase, isoprenoid synthase or mevalonate synthesis-encoding nucleotide sequence is operably linked to an inducible promoter, an inducer is added to the culture medium; and, after a suitable time, the compound is isolated from the organic layer overlaid on the culture medium.

In some embodiments, the compound or product, for example, an isoprenoid compound will be separated from other products which may be present in the organic layer. Separation of the compound from other products that may be present in the organic layer is readily achieved using, e.g., standard chromatographic techniques.

Methods of culturing the host cells, separating products, and isolating the desired product or products are known to one of skill in the an and are discussed further herein.

In some embodiments, the compound, for example, an isoprenoid or isoprenoid compound is produced in a genetically modified host cell at a level that is at least about 2-fold, at least about 5-fold, at least about 10-fold, at least about 25-fold, at least about 50-fold, at least about 100-fold, at least about 500-fold, at least about 1000-fold, at least about 2000-fold, at least about 3000-fold, at least about 4000-fold, at least about 5000-fold, or at least about 10,000-fold, or more, higher than the level of the isoprenoid or isoprenoid precursor compound produced in an unmodified host cell that produces the isoprenoid or isoprenoid precursor compound via the same biosynthetic pathway.

In some embodiments, the compound, for example, an isoprenoid compound is pure, e.g., at least about 40% pure, at least about 50% pure, at least about 60% pure, at least about 70% pure, at least about 80% pure, at least about 90% pure, at least about 95% pure, at least about 98%, or more than 98% pure. “Pure” in the context of an isoprenoid compound refers to an isoprenoid compound that is free from other isoprenoid compounds, portions of compounds, contaminants, and unwanted byproducts, for example.

Examples of products contemplated herein include hydrocarbon products and hydrocarbon derivative products. A hydrocarbon product is one that consists of only hydrogen molecules and carbon molecules. A hydrocarbon derivative product is a hydrocarbon product with one or more heteroatoms, wherein the heteroatom is any atom that is not hydrogen or carbon. Examples of heteroatoms include, but are not limited to, nitrogen, oxygen, sulfur, and phosphorus. Some products can be hydrocarbon-rich, wherein, for example, at least 50%, at least 60%, at least 70%, at least 80%, at least 90%, or at least 95% of the product by weight is made up of carbon and hydrogen.

One exemplary group of hydrocarbon products are isoprenoids. Isoprenoids (including terpenoids) are derived from isoprene subunits, but are modified, for example, by the addition of heteroatoms such as oxygen, by carbon skeleton rearrangement, and by alkylation. Isoprenoids generally have a number of carbon atoms which is evenly divisible by five, but this is not a requirement as “irregular” terpenoids are known to one of skill in the an. Carotenoids, such as carotenes and xanthophylls, are examples of isoprenoids that are useful products. A steroid is an example of a terpenoid. Examples of isoprenoids include, but are not limited to, hemiterpenes (C5), monoterpenes (C10), sesquiterpenes (C15), diterpenes (C20), triterpenes (C30), tetraterpenes (C40), polyterpenes (Cₙ, wherein “n” is equal to or greater than 45), and their derivatives. Other examples of isoprenoids include, but are not limited to, limonene, 1,8-cineole, α-pinene, camphene, (+)-sabinene, myrcene, abietadiene, taxadiene, farnesyl pyrophosphate, fusicoccadiene, amorphadiene, (E)-α-bisabolene, zingiberene, or diapophytoene, and their derivatives.

Products, for example fuel products, comprising hydrocarbons, may be precursors or products conventionally derived from crude oil, or petroleum, such as, but not limited to, liquid petroleum gas, naptha (ligroin), gasoline, kerosene, diesel, lubricating oil, heavy gas, coke, asphalt, tar, and waxes.

Useful products include, but are not limited to, terpenes and terpenoids as described above. An exemplary group of terpenes are diterpenes (C20). Diterpenes are hydrocarbons that can be modified (e.g. oxidized, methyl groups removed, or cyclized); the carbon skeleton of a diterpene can be rearranged, to form, for example, terpenoids, such as fusicoccadiene. Fusicoccadiene may also be formed, for example, directly from the isoprene precursors, without being bound by the availability of diterpene or GGDP. Genetic modification of organisms, such as algae, by the methods described herein, can lead to the production of fusicoccadiene, for example, and other types of terpenes, such as limonene, for example. Genetic modification can also lead to the production of modified terpenes, such as methyl squalene or hydroxylated and/or conjugated terpenes such as paclitaxel.

Other useful products can be, for example, a product comprising a hydrocarbon obtained from an organism expressing a diterpene synthase. Such exemplary products include cot-kaurene, casbene, and fusicoccadiene, and may also include fuel additives.

In some embodiments, a product (such as a fuel product) contemplated herein comprises one or more carbons derived from an inorganic carbon source. In some embodiments, at least 10%, at least 20%, at least 30%, at least 40%, at least 50%, at least 60%, at least 70%, at least 80%, at least 90%, at least 95%, or at least 99% of the carbons of a product as described herein are derived from an inorganic carbon source. Examples of inorganic carbon sources include, but are not limited to, carbon dioxide, carbonate, bicarbonate, and carbonic acid. The product can be, for example, an organic molecule with carbons from an inorganic carbon source that were fixed during photosynthesis.

The products produced by the present disclosure may be naturally, or non-naturally (e.g., as a result of transformation) produced by the host cell(s) and/or organism(s) transformed. For example, products not naturally produced by algae may include non-native terpenes/terpenoids such as fusicoccadiene or limonene. A product naturally produced in algae may be a terpene such as a carotenoid (for example, beta-carotene). The host cell may be genetically modified, for example, by transformation of the cell with a sequence encoding a protein, wherein expression of the protein results in the secretion of a naturally or a non-naturally produced product or products. The product may be a molecule not found in nature.

Examples of products include petrochemical products, precursors of petrochemical products, fuel products, petroleum products, precursors of petroleum products, and all other substances that may be useful in the petrochemical industry. The product may be used for generating substances, or materials, useful in the petrochemical industry. The products may be used in a combustor such as a boiler, kiln, dryer or furnace. Other examples of combustors are internal combustion engines such as vehicle engines or generators, including gasoline engines, diesel engines, jet engines, and other types of engines. In one embodiment, a method herein comprises combusting a refined or “upgraded” composition. For example, combusting a refined composition can comprise inserting the refined composition into a combustion engine, such as an automobile engine or a jet engine. Products described herein may also be used to produce plastics, resins, fibers, elastomers, pharmaceuticals, nutraceuticals, lubricants, and gels, for example.

Useful products can also include isoprenoid precursors. Isoprenoid precursors are generated by one of two pathways; the mevalonate pathway or the methylerythritol phosphate (MEP) pathway. Both pathways generate dimethylallyl pyrophosphate (DMAPP) and isopentyl pyrophosphate (IPP), the common C5 precursor for isoprenoids. The DMAPP and IPP are condensed to form geranyl-diphosphate (GPP), or other precursors, such as farnesyl-diphosphate (FPP) or geranylgeranyl-diphosphate (GGPP), from which higher isoprenoids are formed.

Useful products can also include small alkanes (for example, 1 to approximately 4 carbons) such as methane, ethane, propane, or butane, which may be used for heating (such as in cooking) or making plastics. Products may also include molecules with a carbon backbone of approximately 5 to approximately 9 carbon atoms, such as naptha or ligroin, or their precursors. Other products may include molecules with a carbon background of about 5 to about 12 carbon atoms, or cycloalkanes used as gasoline or motor fuel. Molecules and aromatics of approximately 10 to approximately 18 carbons, such as kerosene, or its precursors, may also be useful as products. Other products include lubricating oil, heavy gas oil, or fuel oil, or their precursors, and can contain alkanes, cycloalkanes, or aromatics of approximately 12 to approximately 70 carbons. Products also include other residuals that can be derived from or found in crude oil, such as coke, asphalt, tar, and waxes, generally containing multiple rings with about 70 or more carbons, and their precursors.

Modified organisms can be grown, in some embodiments in the presence of CO₂, to produce a desired polypeptide. In some embodiments, the products produced by the modified organism are isolated or collected. Collected products, such as terpenes and terpenoids, may then be further modified, for example, by refining and/or cracking to produce fuel molecules or components.

The various products may be further refined to a final product for an end user by a number of processes. Refining can, for example, occur by fractional distillation. For example, a mixture of products, such as a mix of different hydrocarbons with various chain lengths may be separated into various components by fractional distillation.

Refining may also include any one or more of the following steps, cracking, unifying, or altering the product. Large products, such as large hydrocarbons (e.g. ≧C10), may be broken down into smaller fragments by cracking. Cracking may be performed by heat or high pressure, such as by steam, visbreaking, or coking. Products may also be refined by visbreaking for example by thermally cracking large hydrocarbon molecules in the product by heating the product in a furnace. Refining may also include coking, wherein a heavy, almost pure carbon residue is produced. Cracking may also be performed by catalytic means to enhance the rate of the cracking reaction by using catalysts such as, but not limited to, zeolite, aluminum hydrosilicate, bauxite, or silica-alumina. Catalysis may be by fluid catalytic cracking, whereby a hot catalyst, such as zeolite, is used to catalyze cracking reactions. Catalysis may also be performed by hydrocracking, where lower temperatures are generally used in comparison to fluid catalytic cracking. Hydrocracking can occur in the presence of elevated partial pressure of hydrogen gas. Products may be refined by catalytic cracking to generate diesel, gasoline, and/or kerosene.

The products may also be refined by combining them in a unification step, for example by using catalysts, such as platinum or a platinum-rhenium mix. The unification process can produce hydrogen gas, a by-product, which may be used in cracking.

The products may also be refined by altering, rearranging, or restructuring hydrocarbons into smaller molecules. There are a number of chemical reactions that occur in catalytic reforming processes which are known to one of ordinary skill in the arts. Catalytic reforming can be performed in the presence of a catalyst and a high partial pressure of hydrogen. One common process is alkylation. For example, propylene and butylene are mixed with a catalyst such as hydrofluoric acid or sulfuric acid, and the resulting products are high octane hydrocarbons, which can be used to reduce knocking in gasoline blends.

The products may also be blended or combined into mixtures to obtain an end product. For example, the products may be blended to form gasoline of various grades, gasoline with or without additives, lubricating oils of various weights and grades, kerosene of various grades, jet fuel, diesel fuel, heating oil, and chemicals for making plastics and other polymers. Compositions of the products described herein may be combined or blended with fuel products produced by other means.

Some products produced from the host cells of the disclosure, especially after refining, will be identical to existing petrochemicals, i.e. contain the same chemical structure. For instance, crude oil contains the isoprenoid pristane, which is thought to be a breakdown product of phytol, which is a component of chlorophyll. Some of the products may not be the same as existing petrochemicals. However, although a molecule may not exist in conventional petrochemicals or refining, it may still be useful in these industries. For example, a hydrocarbon could be produced that is in the boiling point range of gasoline, and that could be used as gasoline or an additive, even though the hydrocarbon does not normally occur in gasoline.

A product herein can be described by its Carbon Isotope Distribution (CID). At the molecular level, a CID is the statistical likelihood of a single carbon atom within a molecule to be one of the naturally occurring carbon isotopes (for example, ¹²C, ¹³C, or ¹⁴C). At the bulk level of a product, a CID may be the relative abundance of naturally occurring carbon isotopes (for example, ¹²C, ¹³C, or ¹⁴C) in a compound containing at least one carbon atom. It is noted that the CID of a fossil fuel may differ based on its source. For example, with CID(fos), the CID of carbon in a fossil fuel, such as petroleum, natural gas, and coal is distinguishable from the CID(atm), the CID of carbon in current atmospheric carbon dioxide. Additionally, the CID(photo-atm) refers to the CID of a carbon-based compound made by photosynthesis in recent history where the source of inorganic carbon was carbon dioxide in the atmosphere. Also, CID(photo-fos) refers to the CID of a carbon based compound made by photosynthesis in recent history where the source of substantially all of the inorganic carbon was carbon dioxide produced by the burning of fossil fuels (for example, coal, natural gas, and/or petroleum). The exact distribution is also a characteristic of 1) the type of photosynthetic organism that produced the molecule, and 2) the source of inorganic carbon. These isotope distributions can be used to define the composition of photosynthetically-derived fuel products. Carbon isotopes are unevenly distributed among and within different compounds and the isotopic distribution can reveal information about the physical, chemical, and metabolic processes involved in carbon transformation. The overall abundance of ¹³C relative to ¹²C in a photosynthetic organism is often less than the overall abundance of ¹³C relative to ¹²C in atmospheric carbon dioxide, indicating that carbon isotope discrimination occurs in the incorporation of carbon dioxide into photosynthetic biomass.

A product, either before or after refining, can be identical to an existing petrochemical. Some of the fuel products may not be the same as existing petrochemicals. In one embodiment, a fuel product is similar to an existing petrochemical, except for the carbon isotope distribution. For example, it is believed that no fossil fuel petrochemicals have a δ¹³C distribution of less than −32%, whereas fuel products as described herein can have a δ¹³C distribution of less than −32%, less than −35%, less than −40%, less than −45%, less than −50%, less than −55%, or less than −60%. In another embodiment, a fuel product or composition is similar but not the same as an existing fossil fuel petrochemical and has a δ¹³C distribution of less than −32%, less than −35%, less than −40%, less than −45%, less than −50%, less than −55%, or less than −60%.

A fuel product can be a composition comprising, for example, hydrogen and carbon molecules, wherein the hydrogen and carbon molecules are at least about 80% of the atomic weight of the composition, and wherein the δ¹³C distribution of the composition is less than about −32%. For some fuel products described herein, the hydrogen and carbon molecules are at least 90% of the atomic weight of the composition. For example, a biodiesel or fatty acid methyl ester (which has less than 90% hydrogen and carbon molecules by weight) may not be part of the composition. In still other compositions, the hydrogen and carbon molecules are at least 95 or at least 99% of the atomic weight of the composition. In yet other compositions, the hydrogen and carbon molecules are 100% of the atomic weight of the composition. In some embodiments, the composition is a liquid. In other embodiments, the composition is a fuel additive or a fuel product.

Also described herein is a fuel product comprising a composition comprising: hydrogen and carbon molecules, wherein the hydrogen and carbon molecules are at least 80% of the atomic weight of the composition, and wherein the δ¹³C distribution of the composition is less than −32%; and a fuel component In some embodiments, the δ¹³C distribution of the composition is less than about −35%, less than about −40%, less than about −45%, less than about −50%, less than about −55%, or less than about −60%. In some embodiments, the fuel component of the composition is a blending fuel, for example, a fossil fuel, gasoline, diesel, ethanol, jet fuel, or any combination thereof. In still other embodiments, the blending fuel has a δ¹³C distribution of greater than −32%. For some fuel products described herein, the fuel component is a fuel additive which may be MTBE, an anti-oxidant, an antistatic agent, a corrosion inhibitor, or any combination thereof. A fuel product as described herein may be a product generated by blending a fuel product as described and a fuel component. In some embodiments, the fuel product has a δ¹³C distribution of greater than −32%. In other embodiments, the fuel product has a δ¹³C distribution of less than −32%. For example, an oil composition extracted from an organism can be blended with a fuel component prior to refining (for example, cracking) in order to generate a fuel product as described herein. A fuel component, can be a fossil fuel, or a mixing blend for generating a fuel product. For example, a mixture for fuel blending may be a hydrocarbon mixture that is suitable for blending with another hydrocarbon mixture to generate a fuel product. For example, a mixture of light alkanes may not have a certain octane number to be suitable for a type of fuel, however, it can be blended with a high octane mixture to generate a fuel product. In another example, a composition with a δ¹³C distribution of less than −32% is blended with a hydrocarbon mixture for fuel blending to create a fuel product. In some embodiments, the composition or fuel component alone are not suitable as a fuel product, however, when combined, they are useful as a fuel product. In other embodiments, either the composition or the fuel component or both individually are suitable as a fuel product. In yet another embodiment, the fuel component is an existing petroleum product, such as gasoline or jet fuel. In other embodiments, the fuel component is derived from a renewable resource, such as bioethanol, biodiesel, and biogasoline.

Oil compositions, derived from biomass obtained from a host cell, can be used for producing high-octane hydrocarbon products. Thus, one embodiment describes a method of forming a fuel product, comprising: obtaining an upgraded oil composition, cracking the oil composition, and blending the resulting one or more light hydrocarbons, having 4 to 12 carbons and an Octane number of 80 or higher, with a hydrocarbon having an Octane number of 80 or less. The hydrocarbons having an Octane number of 80 or less are, for example, fossil fuels derived from refining crude oil.

The biomass feedstock obtained from a host organism can be modified or tagged such that the light hydrocarbon products can be identified or traced back to their original feedstock. For example, carbon isotopes can be introduced into a biomass hydrocarbon in the course of its biosynthesis. The tagged hydrocarbon feedstock can be subjected to the refining processes described herein to produce a light hydrocarbon product tagged with a carbon isotope. The isotopes allow for the identification of the tagged products, either atone or in combination with other untagged products, such that the tagged products can be traced back to their original biomass feedstocks.

TABLE A
Examples of Enzymes Involved in the Isoprenoid Pathway

| Synthase | Source | NCBI protein ID |
|---|---|---|
| Limonene | M. spicata | 2ONH_A |
| Cineole | S. officinalis | AAC26016 |
| Pinene | A. grandis | AAK83564 |
| Camphene | A. grandis | AAB70707 |
| Sabinene | S. officinalis | AAC26018 |
| Myrcene | A. grandis | AAB71084 |
| Abietadiene | A. grandis | Q38710 |
| Taxadiene | T. brevifolia | AAK83566 |
| FPP | G. gallus | P08836 |
| Amorphadiene | A. annua | AAF61439 |
| Bisabolene | A. grandis | O81086 |
| Diapophytoene | S. aureus | |
| Diapophytoene desaturase | S. aureus | |
| GPPS-LSU | M. spicata | AAF08793 |
| GPPS-SSU | M. spicata | AAF08792 |
| GPPS | A. thaliana | CAC16849 |
| GPPS | C. reinhardtii | EDP05515 |
| FPP | E. coli | NP_414955 |
| FPP | A. thaliana | NP_199588 |
| FPP | A. thaliana | NP_193452 |
| FPP | C. reinhardtii | EDP03194 |
| IPP isomerase | E. coli | NP_417365 |
| IPP isomerase | H. pluvialis | ABB80114 |
| Limonene | L. angustifolia | ABB73044 |
| Monoterpene | S. lycopersicum | AAX69064 |
| Terpinolene | O. basilicum | AAV63792 |
| Myrcene | O. basilicum | AAV63791 |
| Zingiberene | O. basilicum | AAV63788 |
| Myrcene | Q. ilex | CAC41012 |
| Myrcene | P. abies | AAS47696 |
| Myrcene, ocimene | A. thaliana | NP_179998 |
| Myrcene, ocimene | A. thaliana | NP_567511 |
| Sesquiterpene | Z. mays; B73 | AAS88571 |
| Sesquiterpene | A. thaliana | NP_199276 |
| Sesquiterpene | A. thaliana | NP_193064 |
| Sesquiterpene | A. thaliana | NP_193066 |
| Curcumene | P. cablin | AAS86319 |
| Farnesene | M. domestica | AAX19772 |
| Farnesene | C. sativus | AAU05951 |
| Farnesene | C. junos | AAK54279 |
| Farnesene | P. abies | AAS47697 |
| Bisabolene | P. abies | AAS47689 |
| Sesquiterpene | A. thaliana | NP_197784 |
| Sesquiterpene | A. thaliana | NP_175313 |
| GPP Chimera | | |
| GPPS-LSU + SSU fusion | | |
| Geranylgeranyl reductase | A. thaliana | NP_177587 |
| Geranylgeranyl reductase | C. reinhardtii | EDP09986 |
| Chlorophyllidohydrolase | C. reinhardtii | EDP01364 |
| Chlorophyllidohydrolase | A. thaliana | NP_564094 |
| Chlorophyllidohydrolase | A. thaliana | NP_199199 |
| Phosphatase | S. cerevisiae | AAB64930 |
| FPP A118W | G. gallus | |

Percent Sequence Identity

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between two sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

Codon Optimization

One or more codons of an encoding polynucleotide can be “biased” or “optimized” to reflect the codon usage of the host organism. These two terms can be used interchangeably throughout the disclosure. For example, one or more codons of an encoding polynucleotide can be “biased” or “optimized” to reflect chloroplast codon usage (Table A) or nuclear codon usage (Table B) in Chlamydomonas reinhardtii. Most amino acids are encoded by two or more different (degenerate) codons, and it is well recognized that various organisms utilize certain codons in preference to others. Generally, the codon bias selected reflects codon usage of the plant (or organelle therein) which is being transformed with the nucleic acid or acids of the present disclosure. However, the codon bias need not be selected based on a particular organism in which a polynucleotide is to be expressed.

One or more codons can be modified, for example, by a method such as site directed mutagenesis, PCR using a primer that is mismatched for the nucleotide(s) to be changed such that the amplification product is biased to reflect the selected (chloroplast or nuclear) codon usage, or by the de novo synthesis of a polynucleotide sequence such that the change (bias) is introduced as a consequence of the synthesis procedure.

When codon-optimizing a specific gene sequence for expression, factors other than the codon usage may also be taken into consideration. For example, it is typical to avoid restrictions sites, repeat sequences, and potential methylation sites. Most gene synthesis companies utilize computational algorithms to optimize a DNA sequence taking into consideration these and other factors whilst maintaining the codon usage (as defined in the codon usage table) above a user-defined threshold. For example, this threshold may be set such that a codon that is used less than 10% of the time that the corresponding amino acid is present in the proteome would be avoided in the final DNA sequence.

Table A (below) shows the chloroplast codon usage for C. reinhardtii (see U.S. Patent Application Publication No.: 2004/0014174, published Jan. 22, 2004).

TABLE A
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The C. reinhardtii chloroplast genome shows a high AT content and noted codon bias (for example, as described in Franklin S., et al. (2002) Plant J 30:733-744; Mayfield S. P. and Schultz J. (2004) Plant J 37:449-458).

Table B exemplifies codons that are preferentially used in Chlamydomonas nuclear genes.

TABLE B
fields: [triplet] [frequency: per thousand] ([number])
Coding GC 66.30% 1^{st} letter GC 64.80%
2^{nd} letter GC 47.90% 3^{rd} letter GC 86.21%
Nuclear Codon Usage in Chlamydomonas reinhardtii

| | | | |
|---|---|---|---|
| UUU 5.0 (2110) | UCU 4.7 (1992) | UAU 2.6 (1085) | UGU 1.4 (601) |
| UUC 27.1 (11411) | UCC 16.1 (6782) | UAC 22.8 (9579) | UGC 13.1 (5498) |
| UUA 0.6 (247) | UCA 3.2 (1348) | UAA 1.0 (441) | UGA 0.5 (227) |
| UUG 4.0 (1673) | UCG 16.1 (6763) | UAG 0.4 (183) | UGG 13.2 (5559) |
| CUU 4.4 (1869) | CCU 8.1 (3416) | CAU 2.2 (919) | CGU 4.9 (2071) |
| CUC 13.0 (5480) | CCC 29.5 (12409) | CAC 17.2 (7252) | CGC 34.9 (14676) |
| CUA 2.6 (1086) | CCA 5.1 (2124) | CAA 4.2 (1780) | CGA 2.0 (841) |
| CUG 65.2 (27420) | CCG 20.7 (8684) | CAG 36.3 (15283) | CGG 11.2 (4711) |
| AUU 8.0 (3360) | ACU 5.2 (2171) | AAU 2.8 (1157) | AGU 2.6 (1089) |
| AUC 26.6 (11200) | ACC 27.7 (11663) | AAC 28.5 (11977) | AGC 22.8 (9590) |
| AUA 1.1 (443) | ACA 4.1 (1713) | AAA 2.4 (1028) | AGA 0.7 (287) |
| AUG 25.7 (10796) | ACG 15.9 (6684) | AAG 43.3 (18212) | AGG 2.7 (1150) |
| GUU 5.1 (2158) | GCU 16.7 (7030) | GAU 6.7 (2805) | GGU 9.5 (3984) |
| GUC 15.4 (6496) | GCC 54.6 (22960) | GAC 41.7 (17519) | GGC 62.0 (26064) |
| GUA 2.0 (857) | GCA 10.6 (4467) | GAA 2.8 (1172) | GGA 5.0 (2084) |
| GUG 46.5 (19558) | GCG 44.4 (18688) | GAG 53.5 (22486) | GGG 9.7 (4087) |

Generally, the nuclear codon bias selected for purposes of the present disclosure, including, for example, in preparing a synthetic polynucleotide as disclosed herein, can reflect nuclear codon usage of an algal nucleus and includes a codon bias that results in the coding sequence containing greater than 60% G/C content.

Re-Engineering the Genome

In addition to utilizing codon bias as a means to provide efficient translation of a polypeptide, it will be recognized that an alternative means for obtaining efficient translation of a polypeptide in an organism is to re-engineer the genome (e.g., a C. reinhardtii chloroplast or nuclear genome) for the expression of tRNAs not otherwise expressed in the genome. Such an engineered algae expressing one or more exogenous tRNA molecules provides the advantage that it would obviate a requirement to modify every polynucleotide of interest that is to be introduced into and expressed from an algal genome; instead, algae such as C. reinhardtii that comprise a genetically modified genome can be provided and utilized for efficient translation of a polypeptide. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; and Komar et. al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into the genome of the host organism to complement rare or unused tRNA genes in the genome, such as a C. reinhardtii chloroplast genome.

Another Way to Codon Optimize a Sequence for Expression.

An alternative way to optimize a nucleic acid sequence for expression is to use the most frequently utilized codon (as determined by a codon usage table) for each amino acid position. This type of optimization may be referred to as ‘hot codon’ optimization. Should undesirable restriction sites be created by such a method then the next most frequently utilized codon may be substituted in a position such that the restriction site is no longer present. Table C lists the codon that would be selected for each amino acid when using this method for optimizing a nucleic acid sequence for expression in the chloroplast of C. reinhardtii.

TABLE C

| Amino acid | Codon utilized |
|---|---|
| F | TTC |
| L | TTA |
| I | ATC |
| V | GTA |
| S | TCA |
| P | CCA |
| T | ACA |
| A | GCA |
| Y | TAC |
| H | CAC |
| Q | CAA |
| N | AAC |
| K | AAA |
| D | GAC |
| E | GAA |
| C | TGC |
| R | CGT |
| G | GGC |
| W | TGG |
| M | ATG |
| STOP | TAA |

Codon Optimization for the Nucleus of a Desmodesmus, Chlamydomonas, Nannochloropsis, or Scenedesmus Species

To create a codon usage table that can be used to express a gone in the nucleus of several different species, the codon usage frequency of a number of species were analyzed. 30,000 base pairs of DNA sequence corresponding to nuclear protein coding regions for the each of the algal species Scenedesmus sp. (S. dimorphus), Desmodesmus sp. (an unknown Desmodesmus sp.), and Nannochloropsis sp. (N. salina) were used to create a unique nuclear codon usage table for each species. These tables ware then compared to each other and to that of Chlamydomonas reinhardtii; the codon table for the nuclear genome of Chlamydomonas reinhardtii was used as a standard. Any codons that bad very low codon usage for the other algal species but not in Chlamydomonas reinhardtii were fixed at 0 and thus should be avoided in a DNA sequence designed using this codon table (Table D). The following codons should be avoided CGG, CAT, CCG, and TCG. The codon usage able generated is shown in Table D.

TABLE D
Nuclear Codon usage in a Chlamydomonas sp., Scenedesmus sp.,
Desmodesmus sp., and Nannochloropsis sp.
For example, in the first row, the fraction (0.16) is the percentage (16%) of times that a codon
(UUU) is used to code for F (phenylalanine).
(* represents stop codons) (a.a. is amino acid)

| Triplet | a.a. | Fraction | Triplet | a.a. | Fraction | Triplet | a.a. | Fraction | Triplet | a.a. | Fraction |
|---|---|---|---|---|---|---|---|---|---|---|---|
| UUU | F | 0.16 | UCU | S | 0.1 | UAU | Y | 0.1 | UGU | C | 0.1 |
| UUC | F | 0.84 | UCC | S | 0.33 | UAC | Y | 0.9 | UGC | C | 0.9 |
| UUA | L | 0.01 | UCA | S | 0.06 | UAA | * | 0.52 | UGA | * | 0.27 |
| UUG | L | 0.04 | UCG | S | 0 | UAG | * | 0.22 | UGG | W | 1 |
| CUU | L | 0.05 | CCU | P | 0.19 | CAU | H | 0 | CGU | R | 0.11 |
| CUC | L | 0.15 | CCC | P | 0.69 | CAC | H | 1 | CGC | R | 0.77 |
| CUA | L | 0.03 | CCA | P | 0.12 | CAA | Q | 0.1 | CGA | R | 0.04 |
| CUG | L | 0.73 | CCG | P | 0 | CAG | Q | 0.9 | CGG | R | 0 |
| AUU | I | 0.22 | ACU | T | 0.1 | AAU | N | 0.09 | AGU | S | 0.05 |
| AUC | I | 0.75 | ACC | T | 0.52 | AAC | N | 0.91 | AGC | S | 0.46 |
| AUA | I | 0.03 | ACA | T | 0.08 | AAA | K | 0.05 | AGA | R | 0.02 |
| AUG | M | 1 | ACG | T | 0.3 | AAG | K | 0.95 | AGG | R | 0.06 |
| GUU | V | 0.07 | GCU | A | 0.13 | GAU | D | 0.14 | GGU | G | 0.11 |
| GUC | V | 0.22 | GCC | A | 0.43 | GAC | D | 0.86 | GGC | G | 0.72 |
| GUA | V | 0.03 | GCA | A | 0.08 | GAA | E | 0.05 | GGA | G | 0.06 |
| GUG | V | 0.67 | GCG | A | 0.35 | GAG | E | 0.95 | GGG | G | 0.11 |

### EXAMPLES

The following examples are intended to provide illustration of the application of the present disclosure. The following examples are not intended to completely define or otherwise limit the scope of the disclosure.

One of skill in the art will appreciate that many other methods known in the art may be substituted in lieu of the ones specifically described or referenced herein.

The present disclosure provides genes identified from a C. reinhardtii cDNA library that when over expressed in C. reinhardtii confer sodium hypochlorite resistance to the organism. Example 1 describes the construction of the cDNA library.

### Example 1

### cDNA Library Construction

Below is a list of materials used in the construction of the cDNA library.

Plant RNA Reagent (Invitrogen, Cat#12322012)

RNeasy purification kit (QIAGEN, Cat#74106)

RNase-free DNase (QIAGEN, Cat#79254)

Poly(A) Micropurist kit (Ambion, Cat# AM1919)

SuperScript III RT enzyme (Invitrogen, Cat#18080093)

5× First Strand Buffer (included with SuperScript III)

0.1M DTT stock (included with SuperScript III)

10 mM dNTP mixture (Invitrogen, Cat#18427013)

RNaseOUT (Invitrogen, Cat 10777019)

5× Second Strand Buffer (Invitrogen, Cat#10812014)

E. coli DNA polymerase I (Invitrogen, Cat 18010025)

E. coli RNase H (Invitrogen, Cat# AM2293)

E. coli DNA ligase (Invitrogen, Cat#18052019)

10× Annealing Buffer (200 mM Tris HCl pH 7.5, 100 mM MgCl₂, 500 mM NaCl)

T4 DNA polymerase (Invitrogen Cat#18005025)

T4 DNA ligase (Invitrogen)

T4 DNA ligase (New England Biolabs)

Advantage 2 Polymerase mix (Clontech, Cat#639201)

Advantage 2 GC Polymerase mix (Clontech. Cat#639114)

Duplex specific nuclease enzyme (Evrogen)

4× Hybridization buffer (200 mM HEPES pH 7.5, 2M NaCl)

10×DSN Master buffer (100 mM Tris HC pH 8.0, 10 mM MgCl₂, 2 mM DTT)

DSN storage buffer (50 mM Tris HCl pH 8.0)

2×DSN Stop buffer (10 mM EDTA)

PacI restriction enzyme (New England Biolabs)

AseI restriction enzyme (New England Biolabs)

DH10B ElectroMax competent cells (Invitrogen)

Phenol:Chloroform

Chloroform

100% EtOH

100% Isopropanol

100 mg/ml ampicillin stock

50 mg/ml carbenicillin stock

RNase-free water

10 mM dNTP mixture (New England Biolabs)

Taq polymerase (New England Biolabs)

10× Thermopol Buffer (New England Biolabs)

Shrimp Alkaline Phosphatate (USB Scientific)

Exonuclease I (USB Scientific)

QIAGEN Gel Extraction Kit

Below is a table listing the primers used in the construction of the cDNA library.

TABLE 1
Primer 20986
(SEQ ID NO: 1)
(5′-biotin-GGCGGCCGCAAGCAGTGGTATCAACGCAGAGTTAAT
TAATTTTTTTTTTTTTTTTTTTTTTTTTTTTTTTTTTTTTTVN-3')
(V is any one of A, C, or G, N is any one of
A, T, C, or G)
Primer 20987
(SEQ ID NO: 2)
(5'-TCGTCGGGAAGCAGTGGTATCAACGCAGAGTATTAATG-3')
Primer 20988
(SEQ ID NO: 3)
(5'-CATTAATACTCTGCGTTGATACCACTGCTTCCCC-3')
Primer 8018
(SEQ ID NO: 4)
(5'-AAGCAGTGGTATCAACGCAGAGT-3')
Primer 16875
(SEQ ID NO: 5)
(5'-CTACCTTTTGCTGGAAGTGTCATAGC-3')
Primer 16876
(SEQ ID NO: 6)
(5'-CGGCCAGGGGGCCTATGTTCTTTAC-3')
Primer 9250
(SEQ ID NO: 7)
(5'-CGCTGTCTCAAGCAGCATCTAA-3')

Overview of cDNA Library Construction.

Listed below are the steps that were used in the construction of the cDNA library.

1. RNA purification

2. mRNA isolation

3. First strand synthesis by reverse transcription

4. Second strand synthesis and cDNA blunting

5. Amplication of cDNA and normalization

6. Cloning of cDNA into a nuclear expression vector

7. Protocol for screening of transcripts by PCR.

Protocol for RNA Purification.

1. Remove frozen pellet of algal biomass (C. reinhardtii) from −80° C. freezer and weigh. Add 0.5 ml cold (4° C.) Plant RNA Reagent (Invitrogen; USA) per 0.1 gram of frozen pellet. Vortex until the sample is thoroughly resuspended. Incubate the tube containing the lysate horizontally for 5 minutes at room temperature.

2. In a 1.5 ml microfuge tube, add a small amount of zirconium beads and aliquot 500 μl of the lysate. Place in the bead beater for 3 minutes at room temperature.

3. Clarify the solution by centrifugation; spin for 2 minutes at 12,000×g in a bench top centrifuge at room temperature. Transfer the supernatant to an RNase-free 1.5 ml microfuge tube.

4. Add 100 μl 5M NaCl; tap the tube to mix. Add 300 μl chloroform; mix thoroughly by inversion. Centrifuge 10 minutes at 12,000×g (4° C.) to separate the phases.

5. Transfer the top, aqueous phase to an RNase-free 1.5 ml microfuge tube. Add an equal volume of 100% isopropanol to the tube. Mix and incubate 10 minutes at room temperature.

6. Centrifuge 10 minutes at 12,000×g (4° C.) to precipitate the RNA. Carefully decant the supernatant and wash the pellet with 1 ml 70% ethanol. The pellet may be difficult to see at this point; use extra caution to avoid losing the pellet.

7. Centrifuge at mom temperature for 1 minute at 12,000×g. Remove the supernatant by decanting or with a pipette. Spin again briefly to collect residual ethanol and remove with a pipette.

8. Add 30 μl RNase-free water to dissolve the RNA. Immediately proceed to RNA cleanup with the QIAGEN RNeasy Kit.

Steps 9-15 are using the QIAGEN RNeasy Kit (QIAGEN, USA).

9. Dilute the RNA sample to 100 μl with RNase-free water. Add 350 μl Buffer RLT and mix. Finally add 250 μl 100% ethanol, mix, and pipette into an RNeasy spin column. Spin 30 seconds at ≧10,000×g.

10. Discard flow through and add 350 μl Buffer RWI. Spin 30 seconds at ≧10,000×g.

11. Mix 10 μl DNase I with 70 μl Buffer RDD and add directly to the RNeasy spin column membrane. Incubate 15 minutes at room temperature. Add 350 pd Buffer RWI and spin 30 seconds at ≧10,000 ×g.

12. Discard flow through and add 500 μl Buffer RPE. Spin 30 seconds at ≧10,000×g.

13. Repeat wash and spin 2 minutes at ≧10,000×g.

14. Discard flow through and transfer spin column to a clean collection tube. Spin 1 minute at max speed to remove residual Buffer RPE.

15. Elute RNA in 30 μl RNase-free water. Transfer spin column to an RNase-free 1.5 ml microfuge tube and spin 30 seconds at ≧10,000×g. Remove the 30 μl, pipette back on the spin column membrane, and spin a second time for 30 seconds at ≧10,000×g to maximize recovery.

Protocol for mRNA Isolation (Using Ambion Micropurist Kit).

Steps 1-9 are using the Ambion Micropurist Kit (Ambion; USA).

1. Starting with 2-400 μg total RNA, adjust the volume to 250 μl with RNase-free water. Add 250 μl 2× Binding Solution and mix.

2. Pipette the 500 μl mixture into a tube of Oligo(dT) cellulose provided in the Ambion Micropurist Kit. Pipette the mixture up and down several times to resuspend the cellulose and break apart any clumps.

3. Heat the mixture 5 minutes at 65-75° C. in a heat block to denature secondary structures and maximize hybridization with the Oligo(dT). Rock the tube gently for 60 minutes at room temperature.

4. Pellet the Oligo(dT) cellulose by centrifugation; spin 3 minutes at 4,000×g. Aspirate supernatant and set aside on ice.

5. Resuspend the pellet in 500 μl Wash Buffer 1 by vortexing and transfer into the provided spin column. Centrifuge 3 minutes at 4,000×g and discard the flow through.

6. Repeat wash with a second aliquot of 500 μl Wash Buffer 1. Centrifuge 3 minutes at 4,000×g and discard the flow through.

7. Resuspend the pellet in 500 μl Wash Buffer 2 by vortexing. Centrifuge 3 minutes at 4,000×g and discard the flow through.

8. Repeat wash with a second aliquot of 500 μl Wash Buffer 2. Centrifuge 3 minutes at 4,000×g and discard the flow through.

9. Place the spin column in a new RNase-free collection tube. Add 200 μl preheated THE RNA Storage Solution to the Oligo(dT) cellulose, vortex, and spin immediately 2 minutes at 5,000×g.

10. Discard the spin column and ethanol precipitate mRNA. Add 20 μl 5M ammonium acetate, 1 μl glycogen, and 550 μl 100% ethanol. Mix and store in the −80° freezer for 30-60 minutes.

11. Precipitate the cDNA by centrifugation at 4° C.; spin 30 minutes at ≧12,000×g.

12. Carefully decant the supernatant and wash with 1 ml 70% ethanol. Centrifuge 5 minutes at ≧12,000×g.

13. Decant the supernatant and dissolve in 20 μl THE RNA Storage Solution (from Ambion Micropurist Kit). Store at −80° C.

Protocol for First Strand Synthesis by Reverse Transcription.

1. In an RNase-free PCR tube, add the following:
2 μg total RNA or 150 ng mRNA from Step 13 above
0.5 μl 100 μM primer 20986 (SEQ ID NO: 1)
1 μl 10 mM dNTP mixture
Add water to a final volume of 13 μl

2. Incubate 5 minutes at 65° C. in a thermal cycler, remove, and place on ice for 3 minutes.

3. Add in the following order to the PCR tube:
4 μl 5× First Strand Buffer (Invitrogen; USA)
1 μl 0.1M DTT
1 μl RNaseOUT (Invitrogen: USA)
1 μl SuperScript III (Invitrogen; USA)
=20 μl reaction volume

4. Perform the reverse transcription as follows:
Heat controlled lid=72° C.
5 minutes at 42° C.→60 minutes at 52° C.→15 minutes at 70° C.→hold at 4° C.

5. Proceed immediately to second strand synthesis.

Protocol for Second Strand Synthesis and cDNA Termini Blunting.

All reagents must be chilled on ice and reaction setup is to be performed on ice.

1. Add to the 20 μl first strand reaction in the following order:
30 μl 5× Second Strand Buffer (Invitrogen; USA)
91 μl water
3 μl 10 mM dNTP mixture (invitrogen; USA)
1 μl E. coli ligase (Invitrogen; USA)
4 μl E. coli DNA polymerase (Invitrogen; USA)
1 μl E. coli RNase H (Invitrogen; USA)
=150 μl reaction volume

2. Incubate 2 hours at 16° C. with the heated lid turned off.

3. Add 2 μl T4 DNA polymerase to blunt the cDNA termini; incubate reaction 15 minutes at 16° C.

4. After the blunting reaction, add water to a final volume equal to 200 μl and transfer to a 1.5 ml microfuge tube. Add an equal volume of phenol:chloroform and mix vigorously. Spin 2 minutes at 12,000 rpm in a bench top centrifuge.

5. Remove the top, aqueous layer and transfer to a clean 1.5 ml microfuge tube. Add an equal volume of chloroform and mix vigorously. Spin 2 minutes at 12,000 rpm in a bench top centrifuge.

6. Again, remove the top, aqueous layer and transfer to a clean 1.5 ml microfuge tube. Add 0.1 volumes of 3M sodium acetate and 2.5 volumes of 100% ethanol. Mix vigorously and store in the −80° freezer for 30-60 minutes.

7. During the incubation period of Step 6, prepare the 5′ AseI adapter (Steps 7 and 8). To a PCR tube, add the following:
20 μl 100 μM primer 20987 (SEQ ID NO: 2)
20 μl 100 μM primer 20988 (SEQ ID NO: 3)
10 μl 10× Annealing Buffer
50 μl water
=100 μl total volume

Primer 20988 is designed and made such that a phosphate group is attached to the 5′ end of the primer sequence.

8. Heat the mixture to 95° C. for 5 minutes and slow cool to 25° C. over 60 minutes.

9. Precipitate the cDNA (from Step 6) by centrifugation at 4° C.; spin 30 minutes at ≧12,000 rpm.

10. Carefully decant the supernatant and wash with 1 ml 70% ethanol. Centrifuge 5 minutes at ≧12,000 rpm.

11. Repeat ethanol precipitation to remove traces of phenol.

12. Decant the supernatant, aspirate all traces of ethanol, and dissolve the cDNA pellet in 10 μl 5′ AseI adapter from Step 8 above.

13. Ligate blunted cDNA and 5′ AseI adapter in the following reaction:
7.5 μl cDNA-adapter mixture
2 μl 5× T4 DNA ligase reaction buffer (Invitrogen; USA)
0.5 μl T4 DNA ligase (Invitrogen; USA)
=10 μl reaction volume

14. Incubate 18 hours at 16° C., hold at 4° C.

Protocol for Amplification and Normalization of cDNA.

1. Amplify cDNA from Step 14 above by long distance PCR (LD-PCR). In a 100 μl PCR tube, add the following:
2 μl cDNA
70 μl water
20 μl 5× Advantage 2 GC buffer (CLONTECH; USA)
4 μl primer 8018 (SEQ ID NO: 4)
2 μl mM dNTP mixture (CLONTECH; USA)
2 μl Advantage 2 GC polymerase mix (CLONTECH; USA)
=100 μl reaction volume

2. Run the following program on a thermal cycler:

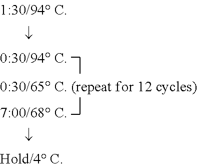

3. Clean up the PCR reaction with a QIAGEN PCR Purification Kit (QIAGEN; USA).

4. Equilibrate 4× hybridization buffer to room temperature prior to normalization.

5. In the first tube of a 4-tube PCR strip, add 1200 ng of cDNA, 4 μl 4× hybridization buffer, and water to a total volume equal to 16 μl. Mix well and aliquot 4 μl to each of the other 3 tubes. Spin briefly to collect the sample in the bottom of the tube.

6. Heat tube to 98° C. for 5 minutes and incubate 5 hours at 68° C. in a thermal cycler.

7. As the end of the 5 hour incubation approaches, prepare the duplex specific nuclease (DSN) enzyme (Evrogen; Russia). Make the following enzyme dilutions (volume of enzyme will depend on the number of samples):
Tube 1=1×DSN enzyme
Tube 2=½×DSN enzyme
Tube 3=¼×DSN enzyme
Tube 4=DSN storage buffer (control)

8. Pre-warm 20 μl 2×DSN master buffer to 68° C. in the thermal cycler. Add 5 μl of 2×DSN master buffer to each tube and incubate 10 minutes at 68° C.

9. Add 1 μl of each of the four DSN enzyme dilutions (from Step 9) to the appropriate tube (i.e. 1× to tube 1, ½× to tube 2, etc); incubate 25 minutes at 68° C. It is very important that the sample temperature does not dip below 68° C. Add the DSN enzyme to the samples while still in the thermal cycler. Spin briefly to collect reaction in the bottom of the tube and immediately return to the thermal cycler.

10. Add 10 μl 2×DSN stop buffer to the reaction and incubate 5 minutes at 68° C. Immediately place on ice and add 20 μl water.

11. Amplify normalized cDNA by LDPCR to estimate the success of normalization.
1 μl normalization reaction (from Step 10)
35.5 μl water
10 μl 5× Advantage 2 GC buffer (CLONTECH; USA)
1.5 μl primer 8018
1 μl 10 mM dNTP mixture (CLONTECH; USA)
1 μl Advantage 2 GC polymerase mix (CLONTECH; USA)
=50 μl reaction volume

12. Run the following program on a thermal cycler

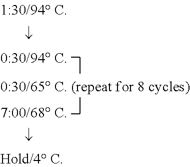

13. Remove 5 μl from the control tube for 1.1% agarose gel analysis and return this reaction only to thermal cycler for an additional 2 cycles. Again remove a sample for agarose gel analysis and perform an additional 2 cycles. Repeat twice more to optimize cDNA amplification conditions and avoid overcycling.

14. Once the amplification conditions have been optimized and the proper DSN enzyme concentration determined, amplify 4-8×100 μl of normalized cDNA using the LDPCR conditions in step 12 for 12 cycles as determined in step 13.

15. Purify normalized cDNA by phenol:chloroform extraction and ethanol precipitation.

Protocol for Cloning Normalized cDNA.

1. Digest normalized cDNA (from Step 15 above) with AseI and PacI.
˜2 μg normalized cDNA
10 μl 10×NEB Buffer #20
1 μl BSA
6 μl AseI
4 μl PacI
Water to a final volume=100 μl

2. Digest at 37° C. for 4-5 hours. Heat kill the enzyme for 20 minutes at 65° C.

3. If necessary, purify digested cDNA by phenol:chloroform extraction and 2× ethanol precipitation to concentrate cDNA into a smaller volume.

4. Run the entire digest on a 1.1% agarose gel. Fractionate the cDNA library excising a gel band that contains only cDNA ≧700 bp. Purify the gel slice with a QIAGEN Gel Extraction Kit.

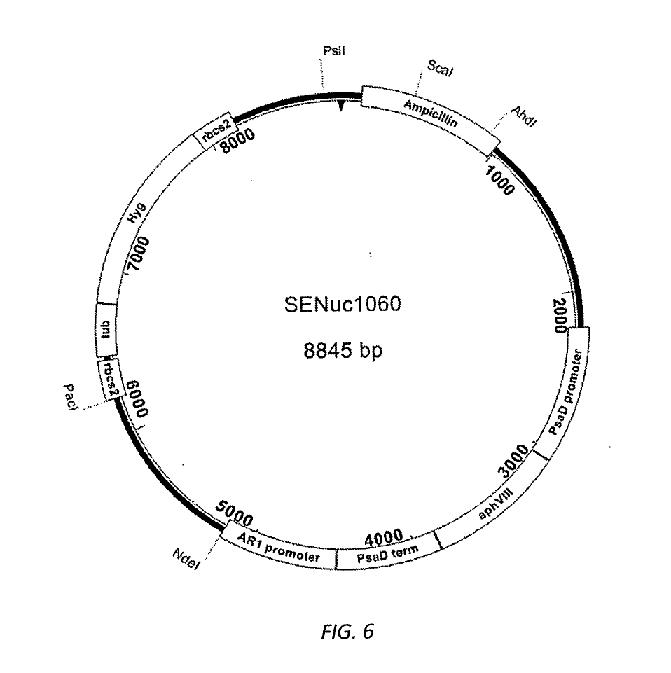

5. Ligate the AseI/PacI digested cDNA with NdeI/PacI digested SENuc1060 (FIG. 6) cDNA overexpression vector. Use a 5:1 insert to vector molar ratio, assuming an average transcript size of 1.5 kb.
21.3 μg cDNA insert
25 μg vector
2 μl 10 T4 DNA ligase buffer (New England Biolabs)
1 μl T4 DNA ligase (New England Biolabs)
Water to a final volume=20 μl

6. Incubate 18 hours at 16° C., hold at 4° C.

7. Transform 0.8 μl of ligation with 20 μl DH10B ElectroMax competent cells. Electroporate and recover in 1 ml of SOC medium for 1 hour at 37° C.

8. Plate the appropriate amount of transformation on LB-Carbenicillin (50 μg/ml) agar plates. Incubate overnight at 37° C. The amount plated will depend on the size of the agar plates—for colony screening, only 100-200 μl needs to be plated.

9. Pick individual colonies for screening cloned cDNA transcripts.

Protocol for Screening of Transcripts by PCR.

1. Inoculate individual colonies from transformation plates into a 96 well plate containing 200 μl LB broth+Ampicillin (100 μg/ml). Grow on an orbital shaker at 37° C. for 2-3 hours.

2. Remove 1 μl of culture and screen by PCR:
2 μl culture
2.5 μl 10× Thermopol Buffer
0.5 μl primer 16875 (SEQ ID NO: 5)
0.5 μl primer 16876 (SEQ ID NO: 6)
0.5 μl DMSO
0.5 μl 10 mM dNTP mixture
0.25 μl Taq polymerase
19.25 μl water
=25 μl reaction volume

3. Run the following program on a thermal cycler:

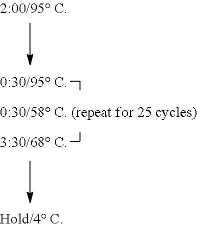

4. Remove 5 μl for sequencing and analyze the remaining PCR reaction by agarose gel electrophoresis.

5. Add 2 μl of Exo/SAP (2:2:5:1, Exonucleasel:Shrimp Alkaline Phosphatase:water:50 mM Tris HCl pH 8) to the 5 μl of reaction for sequencing. Incubate reaction for 15 minutes at 37° C. then 15 minutes at 80° C. in a thermal cycler. Sequence with primer 9250 (SEQ ID NO: 7) to read from the 5′ end of the clone.

### Example 2

### Sodium hypochlorite Screening

A Chlamydomonas reinhardtii cDNA library was cloned, as described above, into an overexpression cassette contained within a construct, which was flanked by genes that confer resistance to hygromycin and paromomycin (used for algal selection) (See FIG. 6). The cloned library was then transformed into Chlamydomonas reinhardtii.

Library DNA was prepared by digesting cDNA cloned into SENuc1060 vector with the restriction enzymes ScaI, AhdI or PsiI followed by heat inactivation of the enzyme. Three independent digests for each restriction enzyme were performed and subsequently combined prior to transformation. For these experiments, all transformations were carried out on C. reinhardtii cc1690 (mt+) cells. Cells were grown and transformed via electroporation. Cells were grown to mid-log phase (approximately 2-3.5×10⁶ cells/ml) in TAP media. Cells were spun down at 3000×g for 10 min. The supernatant was removed and the cells were resuspended in 1 mM HEPES/1 mM MgCl₂ pH 7.4. Cells were then centrifuged in a conical tube for 10 min at 3,000×g. The supernatant was carefully decanted and the wash with HEPES/MgCl₂ was repeated. After the second spin, the supernatant was decanted and 1 mM HEPES/1 mM MgCl₂ buffer was added to yield a final concentration of 3×10⁸ cells. Then 500 mM ATP and 200 mM glutathione to a final concentration of 5 mM and 2 mM respectively was added. 250-1000 ng (in 1-5 μL H₂O) of transformation DNA was mixed with 250 μL of 3×10⁸ cells/mL on ice and transferred to 0.4 cm electroporation cuvettes. Electroporation was performed with the capacitance set at 25 uF and the voltage at 700 V resulting in a time constant of 40 ms. Following electroporation, the cuvette was immediately returned to a room temperature water bath for 10 min. For each transformation, cells were transferred to 10 ml of TAP media+40 mM sucrose and allowed to recover at room temperature for 12-16 hours with continuous shaking. Cells were then harvested by centrifugation at 3000×g for 10 min, the supernatant was discarded, and the pellet was resuspended in 0.5 ml TAP media+40 mM sucrose. The resuspended cells were then plated on solid TAP media+10 μg/mL hygromycin+10 μg/mL paromycin.

Transformants that grew on hygromycin and paromomycin selection (approximately 300,000 individual clones) were scraped from plates and then recovered in 0.5-1.0 L liquid TAP media for 24 hours. This culture was used for turbidostat and flask bleach screening experiments.

For turbidostat experiments, three sets of biological triplicate turbidostats were inoculated with 10 mL of the recovered library and filled with MASM(D) to 60 ml media. One liter MASM(D) media feed bottles for the turbidostats were supplemented with 0, 2.5, and 5 ppm sodium hypochlorite (one set of triplicates at each level of sodium hypochlorite). Cultures were grown under CO₂ and continuously maintained at OD₇₅₀=0.25. Turbidostats were sorted by FACS at weeks 2, 4, and 6 to isolate single colonies.

Preparation of Modified Artificial Seawater Media (MASM)-D.

For 1 liter of MASM-D: add 1.0 g Tris; 2.49 g MgSO₄.7H₂O; 0.6 g KCl; 1.0 g NaNO₃; 0.30 g CaCl₂.2H₂O: 0.05 g KH₂PO₄; 1.0 ml NH₄Cl 0.5M; and 6.0 ml CM Trace Elements Solution to 900 ml deionized water. Dissolve, mix and adjust the pH of the solution to pH 8.0 with a solution of HCl. Adjust the volume to 1 liter with deionized water. Autoclave solution for 60 minutes at 121° C./15 PSI. After autoclaving, let cool to about 55° C. or below and add 10 ml of filter sterilized 1.19M NaHCO₃ to the bottle and mix.

For 1 liter of CM Trace Elements Solution: add 1 g NaEDTA; 0.194 g FeCl₃.6H₂O; 2 ml 182 mM MnCl₂.4H₂O; 2 ml 77 mM ZnCl₂; 1 ml 52 mM Na₂MoO₄.2H₂O; and 1 ml 17 mM CoCl₂.6H₂O to 900 ml deionized water. Dissolve, mix, and adjust the volume to 1 liter with deionized water. Filter sterilize the solution.

In addition, the recovery culture from the library transformation was also grown in a 250 ml flask. Cells (approximately 30-50 mls) from the recovered library were centrifuged for 5 minutes at 3,000 rpm and washed in photosynthetic media.

Washed cells were diluted to 100 ml in the photosynthetic media at a density equal to 1.0×10⁶ cells per milliliter. This culture was challenged with an acute dosage (10 ppm) of sodium hypochlorite on alternating days for one week (3 total dosages). Flasks were then sorted by FACS at the end of the week to isolate single colonies.

Colonies from both turbidostat and flask experiments were lysed by boiling in 10×TE buffer and the cDNA insert was amplified by PCR. Amplification products were sequenced to determine the 5′ end of the transcript cloned into the cDNA overexpression vector. Sequences that generated a full length or in-frame gene were identified using BLAST, which generated a candidate list of 42 genes. Candidates from the turbidostat screening were given BT identifiers; candidates from flask screening were given BF identifiers.

These candidates were subjected to a microtiter assay for initial validation of the sodium hypochlorite tolerance phenotype. Transgenic lines (BT and BF genes) containing candidates identified in selective sodium hypochlorite turbidostats or flasks and wild-type Chlamydomonas reinhardtii were grown to saturation in TAP.

The cultures were harvested by centrifugation and resuspended in photosynthetic media to a final OD₇₅₀ equal to 0.35. Each sodium hypochlorite candidate and wild type control were diluted to OD₇₅₀ as follows: 0.35, 0.325, 0.3, 0.25, 0.2, 0.15, 0.125, and 0.1. Aliquots of 200 microliters were dispensed across individual rows of a clear 96-well assay plate. Sodium hypochlorite was then added at the following concentrations; 0, 10, 15, 25, 35, and 60 ppm. Each concentration was performed in duplicate. Plates were scaled with clear adhesive tape and incubated for 36 hours.

Tolerance to sodium hypochlorite was determined visually by a comparison of the wild type cells and cells overexpressing the sodium hypochlorite candidates. Wells containing cells that had greater resistance to increasing bleach concentrations were viable and remained green as compared to the wild type control; wells containing cells that did not have as much resistance contained dead cells or were clear.

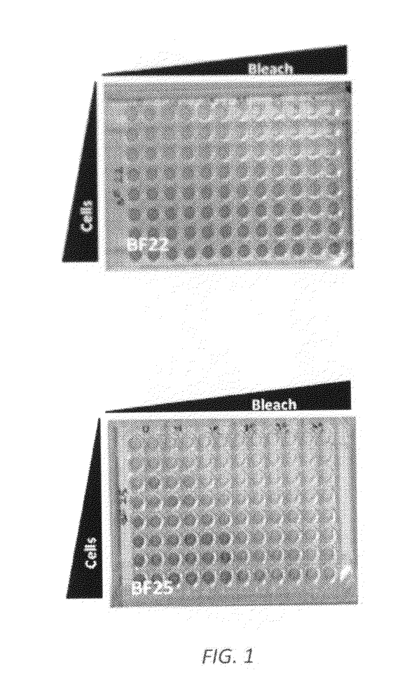

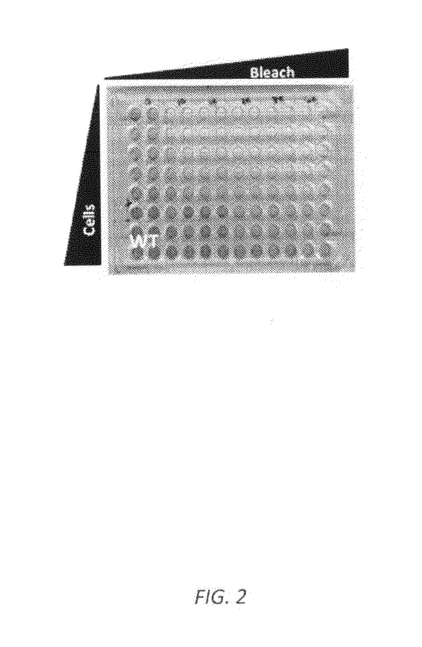

Shown in FIG. 1 is a representative plate of a candidate that passed (BF22) and a candidate that failed (BF25) this validation step, wild-type (FIG. 2) is included for comparison.

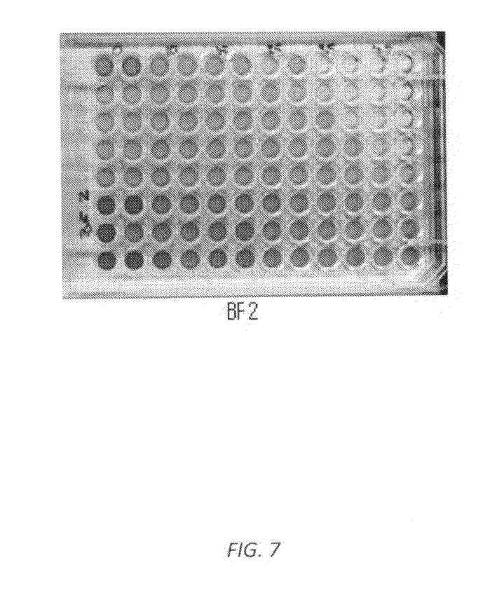

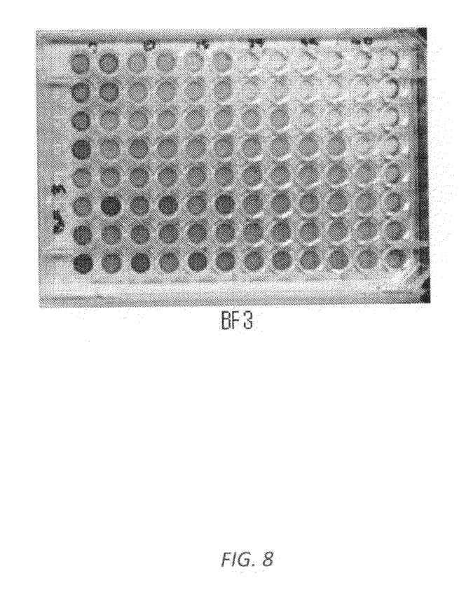

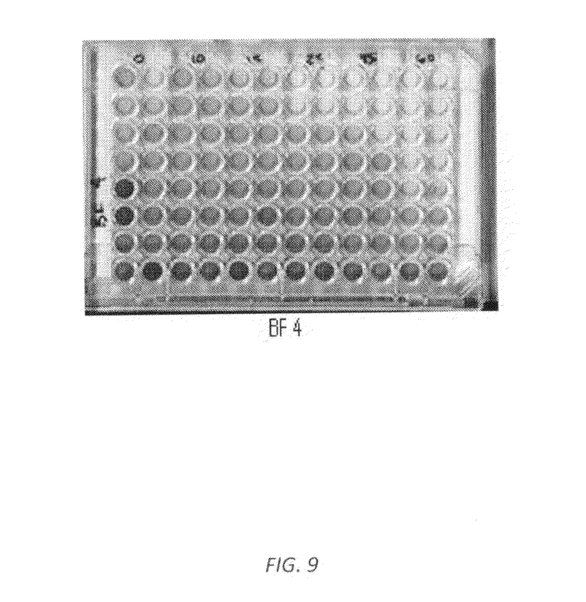

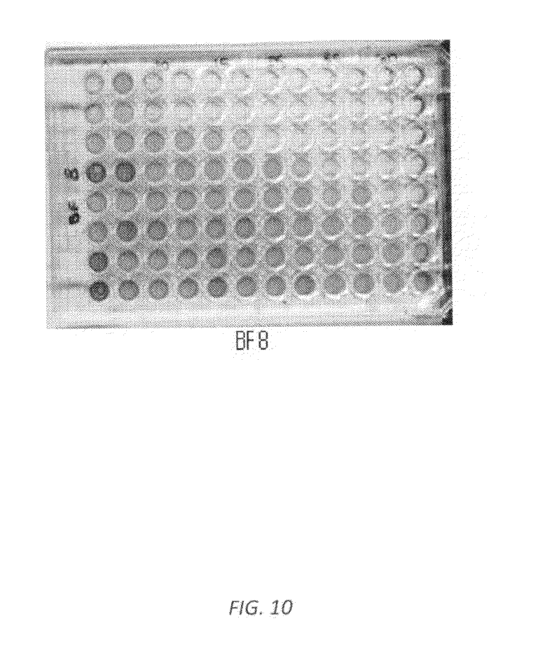

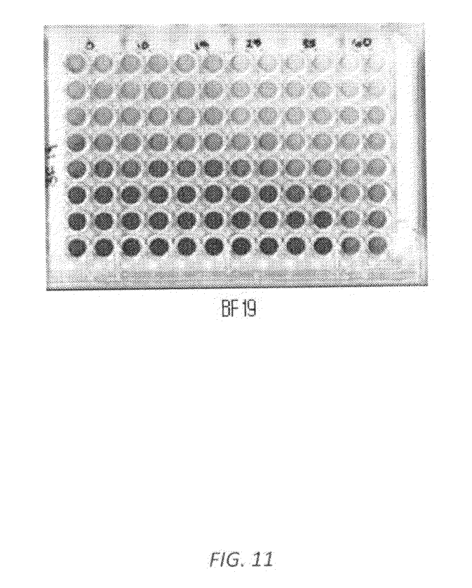

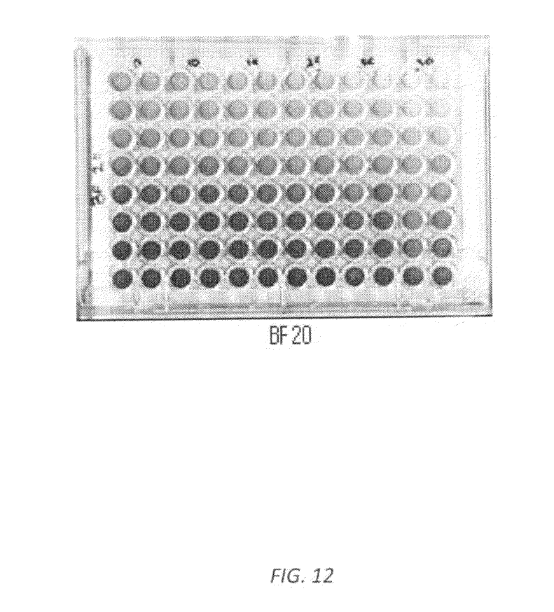

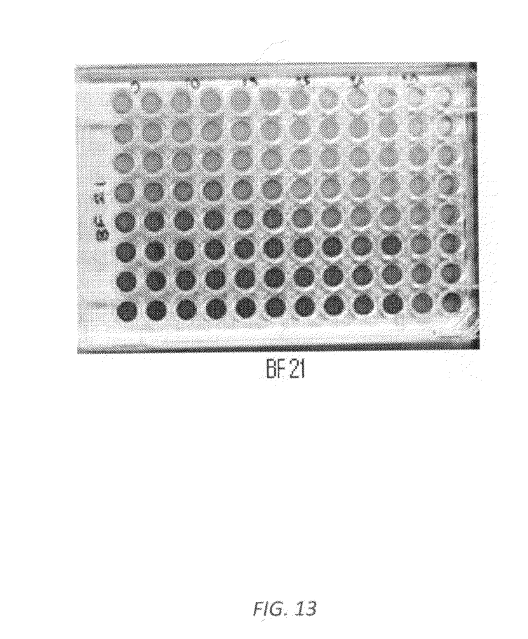

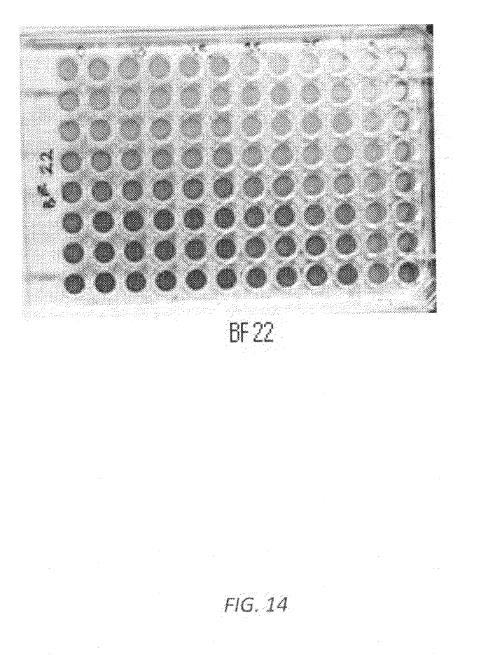

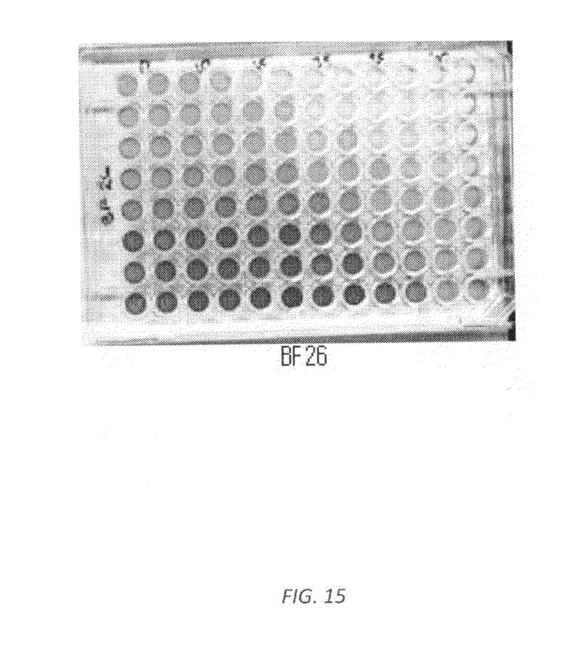

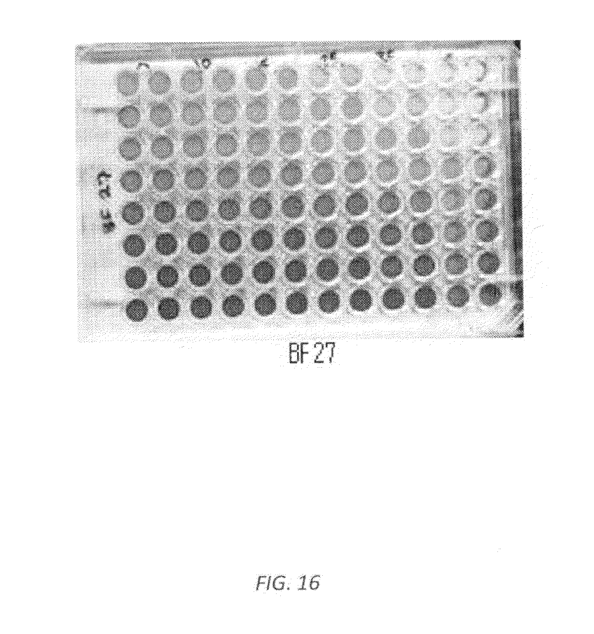

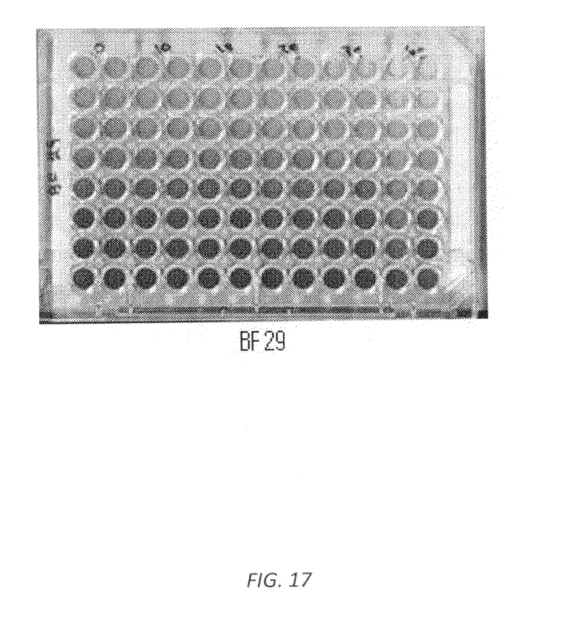

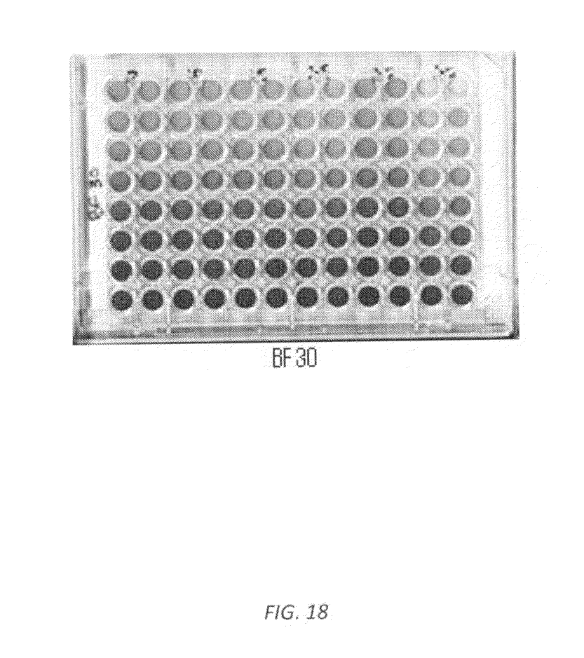

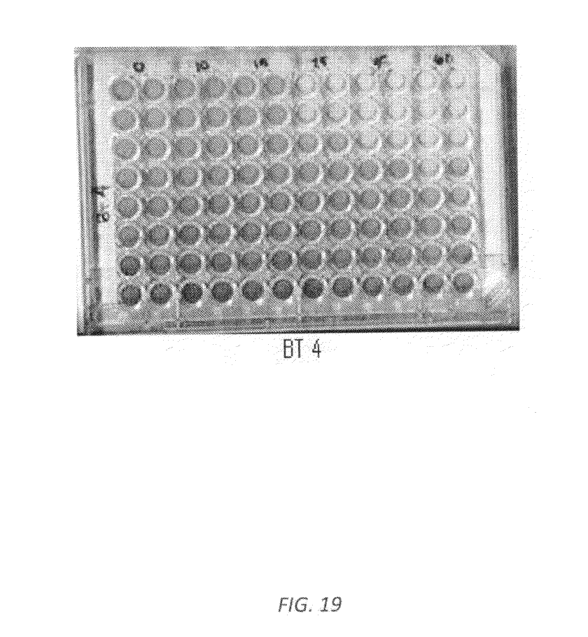

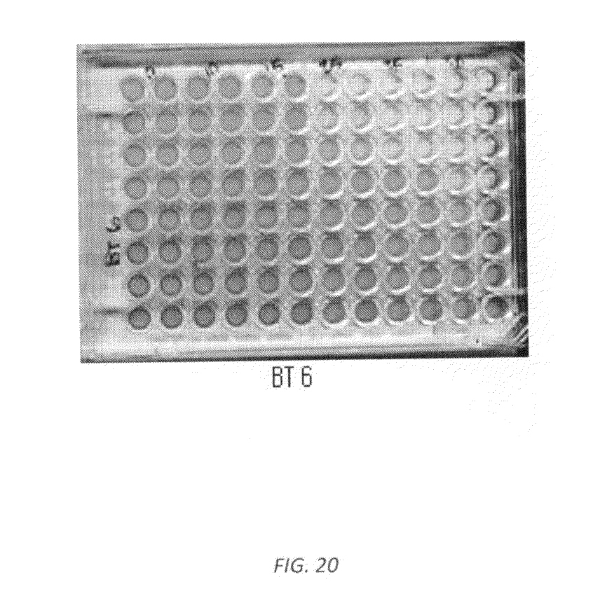

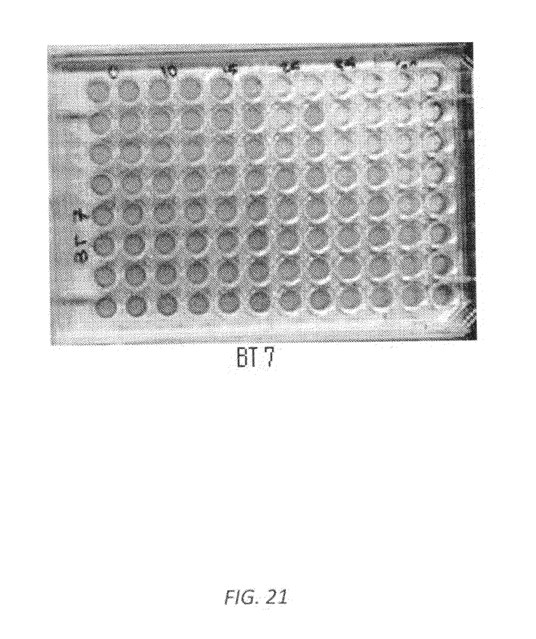

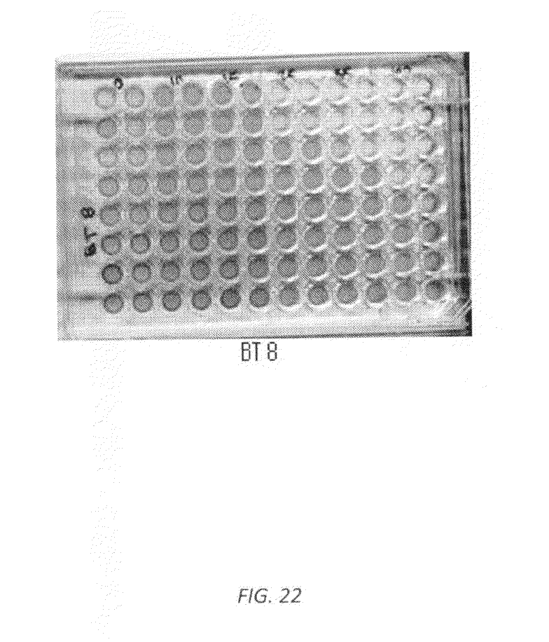

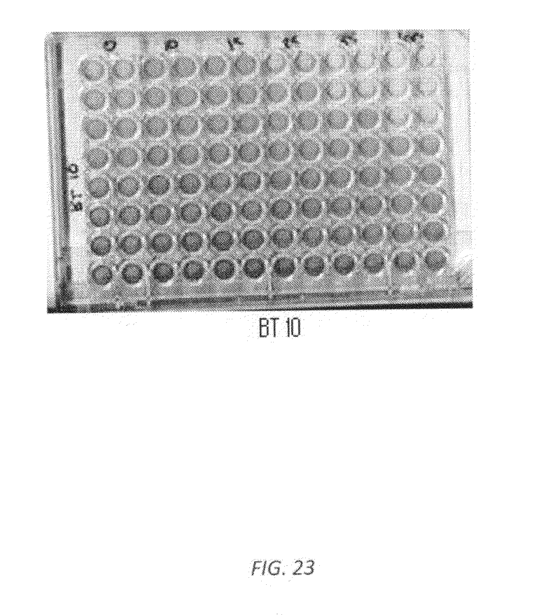

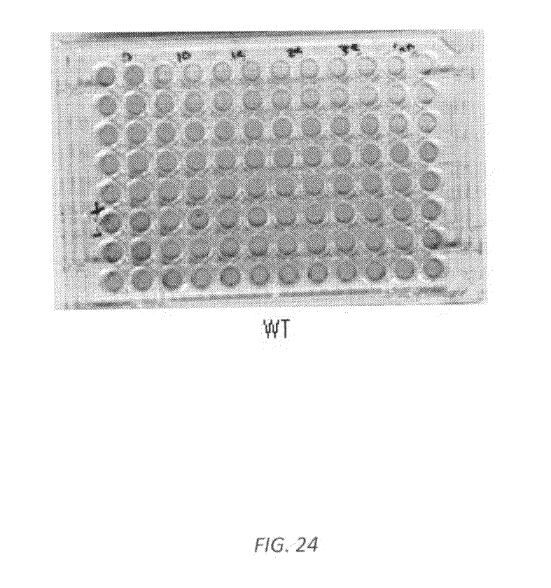

Shown in FIG. 7 to FIG. 24 are the results of the microtiter assay for sodium hypochlorite tolerance for the BF and BT candidates.

Candidates that did not outperform wild-type in this assay were excluded from further validation. Microtiter validation yielded 17 candidates that performed better than wild-type, which are listed in Table 2 below (BF=candidates from flask experiments, BT=candidates from turbidostat experiments).

The genes that provide bleach tolerance in C. reinhardtii are listed in Table 2 along with the Joint Genome Institute (JGI) protein ID (ver. 4) and functional annotation. *BF02 is from JGI ver. 3. **BT06 is a transcript ID as the protein is encoded in the 5′UTR of another annotated transcript.

Also listed in the table are sequence identifier numbers for the gene sequences and for the translated protein sequences.

TABLE 2

| BT/BF | JGI Protein ID | Functional annotation | SEQ ID NO: |
|---|---|---|---|
| BF02 | 187716* | hypothetical protein | SEQ ID NO: 8 |
| BF03 | 56014 | cytochrome C oxidase subunit III | SEQ ID NO: 9 |
| BF04 | 379840 | hypothetical protein | SEQ ID NO: 10 |
| BF08 | 206201 | carbonic anhydrase CAH3 | SEQ ID NO: 11 |
| BF10/BF26 | 60444 | malate dehydrogenase | SEQ ID NO: 12 |
| BF19 | 184222 | NADH:ubiquinone oxidoreductase | SEQ ID NO: 13 |
| BF20 | 206086 | selenoprotein M2 | SEQ ID NO: 14 |
| BF21 | 182441 | blue copper protein | SEQ ID NO: 15 |
| BF22 | 192933 | 26S proteasome regulatory subunit | SEQ ID NO: 16 |
| BF27 | 191516 | mitochondrial substrate carrier | SEQ ID NO: 17 |
| BF29 | 55838 | ribose-5-phosphate isomerase, residues 22-269 | SEQ ID NO: 18 |
| BF30 | 128777 | V-SNARE/VAMP71 | SEQ ID NO: 19 |
| BF32 | 137795 | PAM18 translocase protein | SEQ ID NO: 20 |
| BT06 | 344164** | similar to peroxisomal biogenesis protein from | SEQ ID NO: 21 |
| | | Volvox carteri f. nagariensis | |
| BT07 | 126203 | drought/stress related protein | SEQ ID NO: 22 |
| BT08 | 160392 | chaperonin 20 | SEQ ID NO: 23 |
| BT11 | 137766 | early light-inducible protein, ELI2-1 | SEQ ID NO: 24 |
| BF02 | — | amino acid sequence of SEQ ID NO: 8 | SEQ ID NO: 25 |
| BF03 | — | amino acid sequence of SEQ ID NO: 9 | SEQ ID NO: 26 |
| BF04 | — | amino acid sequence of SEQ ID NO: 10 | SEQ ID NO: 27 |
| BF08 | — | amino acid sequence of SEQ ID NO: 11 | SEQ ID NO: 28 |
| BF10/BF26 | — | amino acid sequence of SEQ ID NO: 12 | SEQ ID NO: 29 |
| BF19 | — | amino acid sequence of SEQ ID NO: 13 | SEQ ID NO: 30 |
| BF20 | — | amino acid sequence of SEQ ID NO: 14 | SEQ ID NO: 31 |
| BF21 | — | amino acid sequence of SEQ ID NO: 15 | SEQ ID NO: 32 |
| BF22 | — | amino acid sequence of SEQ ID NO: 16 | SEQ ID NO: 33 |
| BF27 | — | amino acid sequence of SEQ ID NO: 17 | SEQ ID NO: 34 |
| BF29 | — | amino acid sequence of SEQ ID NO: 18 | SEQ ID NO: 35 |
| BF30 | — | amino acid sequence of SEQ ID NO: 19 | SEQ ID NO: 36 |
| BF32 | — | amino acid sequence of SEQ ID NO: 20 | SEQ ID NO: 37 |
| BT06 | — | amino acid sequence of SEQ ID NO: 21 | SEQ ID NO: 38 |
| BT07 | — | amino acid sequence of SEQ ID NO: 22 | SEQ ID NO: 39 |
| BT08 | — | amino acid sequence of SEQ ID NO: 23 | SEQ ID NO: 40 |
| BT11 | — | amino acid sequence of SEQ ID NO: 24 | SEQ ID NO: 41 |

### Example 3

### Sodium Hypochlorite Radial Diffusion Assay

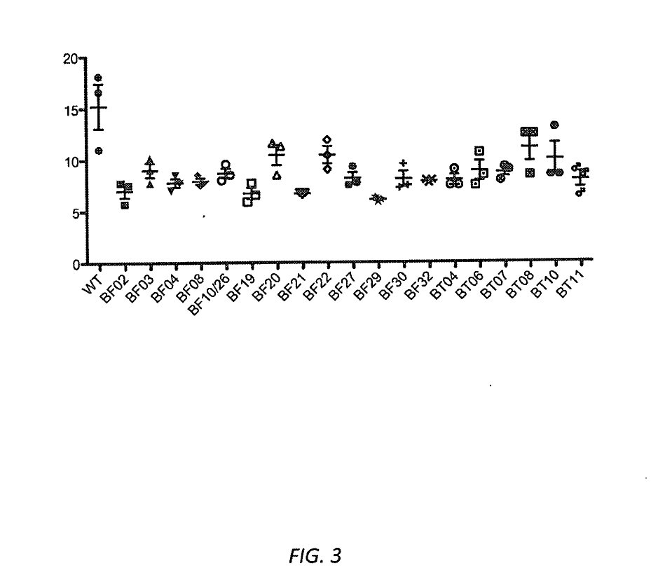

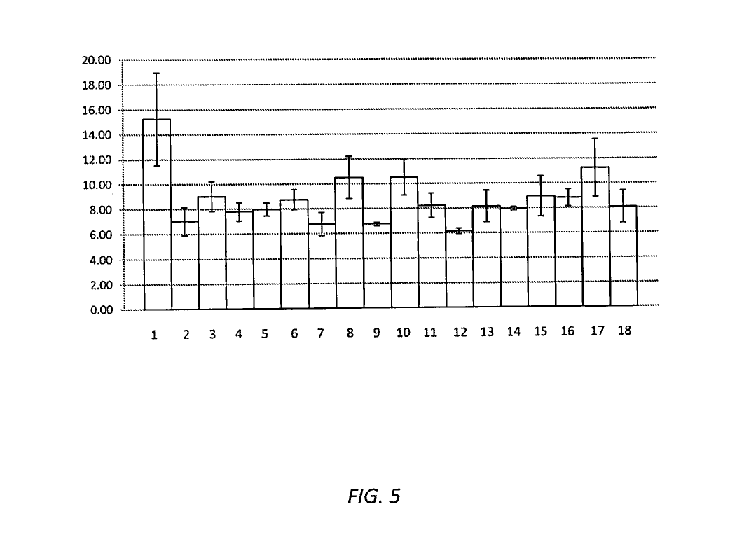

The 17 sodium hypochlorite tolerant candidates were further validated using a radial diffusion assay, which allows the relative sodium hypochlorite tolerance of each candidate to be quantitated. Each candidate was grown in liquid culture (TAP) to mid-log phase. Approximately 3×10⁶ cells were spread onto non-selective agar plates (in triplicate) and allowed to dry. A 100 μl hole was punched in the middle of the plate and 100 μl of a 10% sodium hypochlorite solution was deposited (100,000 ppm). Once the sodium hypochlorite absorbed into the agar, the plates were then placed under constant light for 48 hrs. To quantitate the relative sodium hypochlorite tolerance, the diameter of each circle of inhibition was measured in three directions and averaged for each plate (as shown in Table 3). The area for each plate was calculated from the diameter, then the average area for each candidate was calculated, along with the standard error (as shown in Table 4); data for this experiment is depicted in FIG. 3 and FIG. 5. FIG. 5 represents the average area of inhibition.

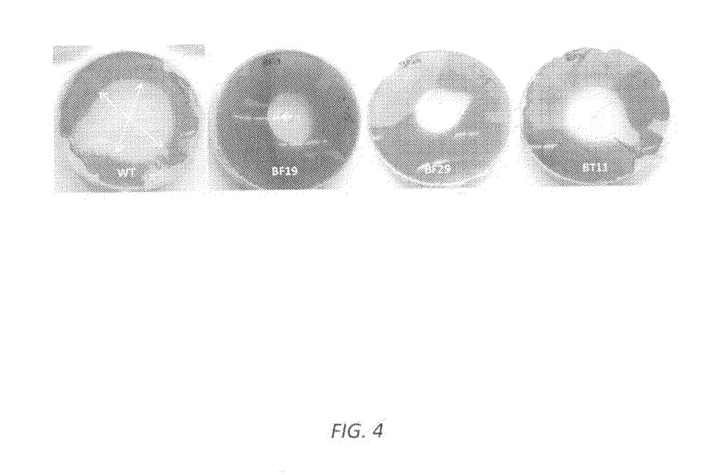

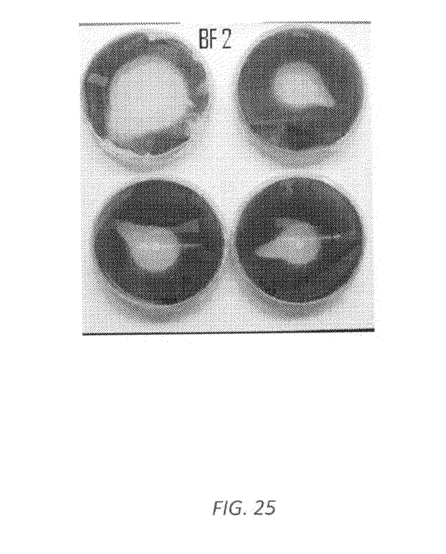

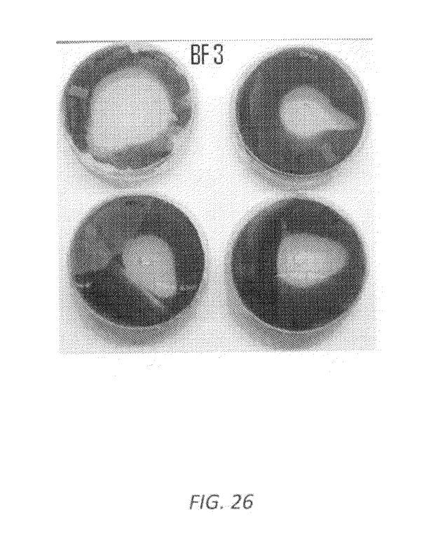

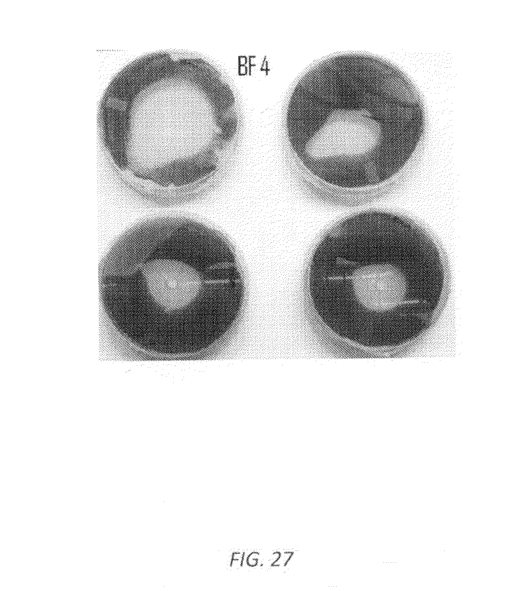

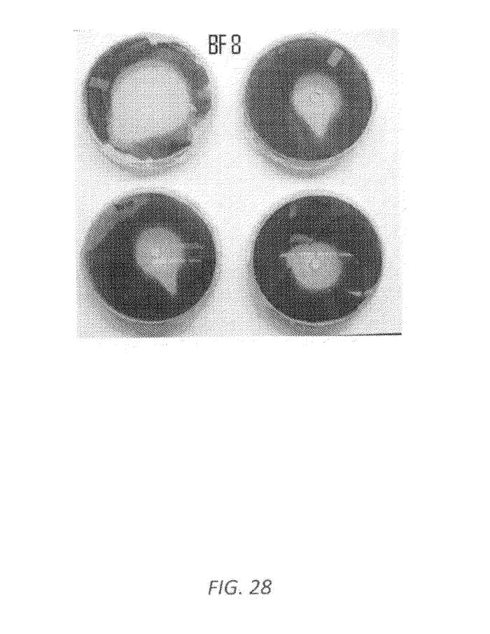

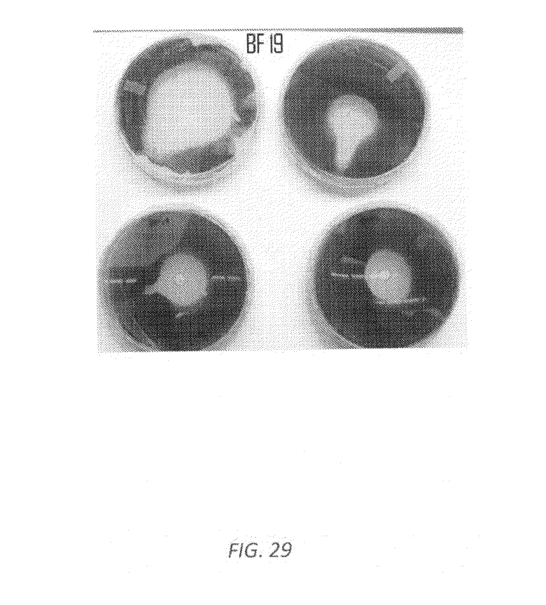

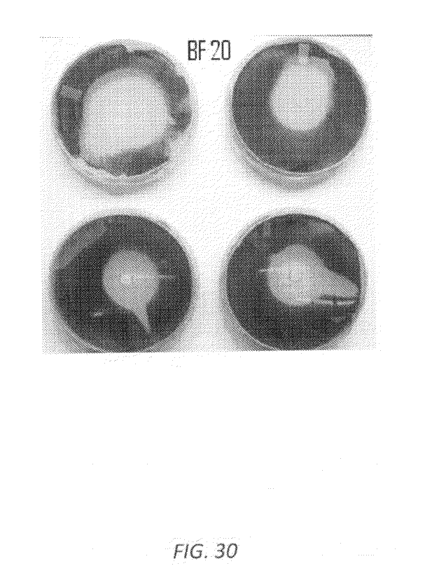

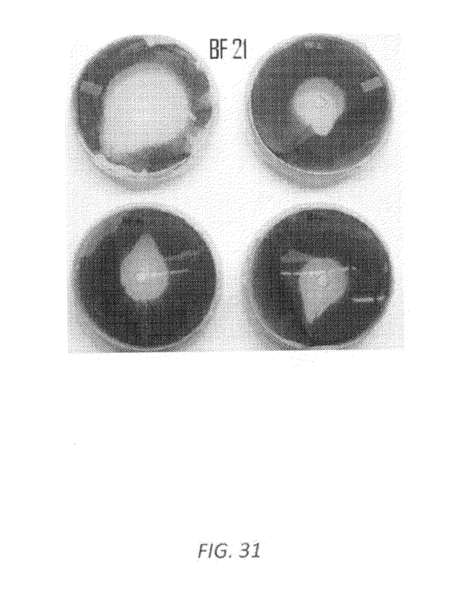

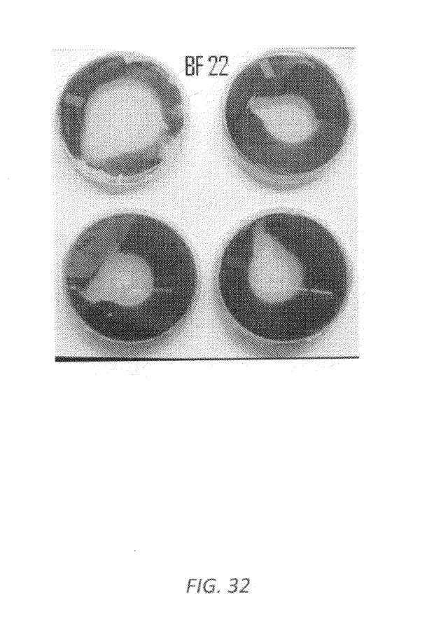

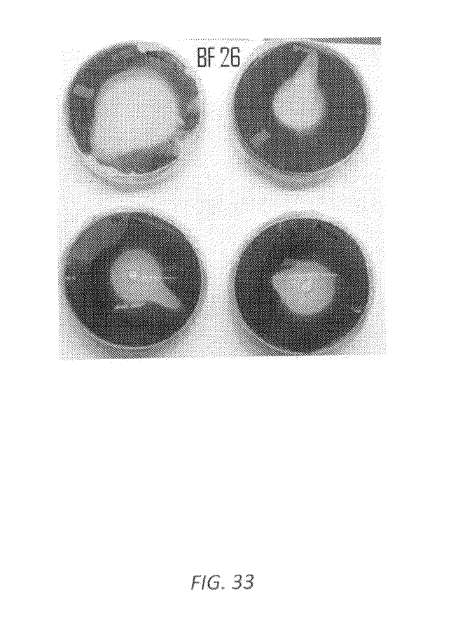

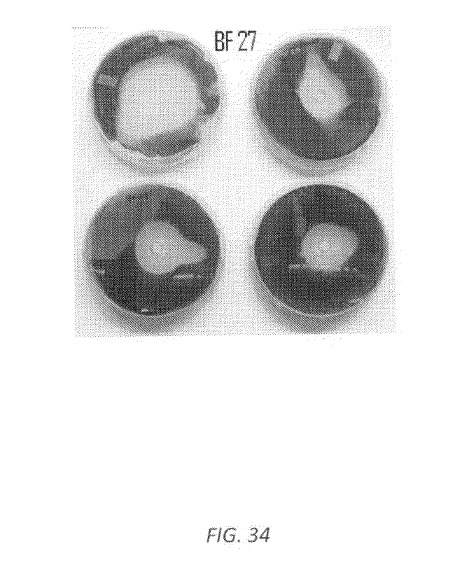

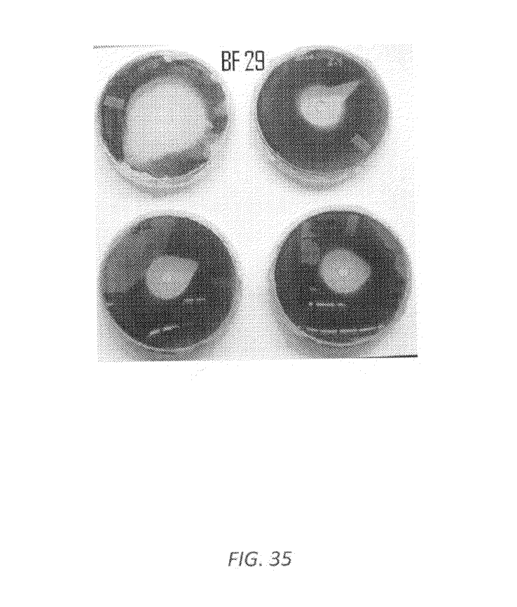

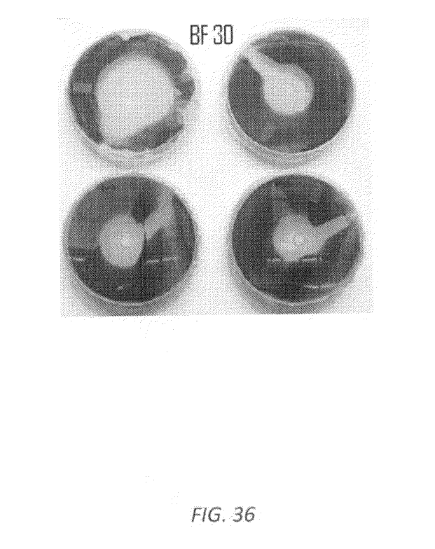

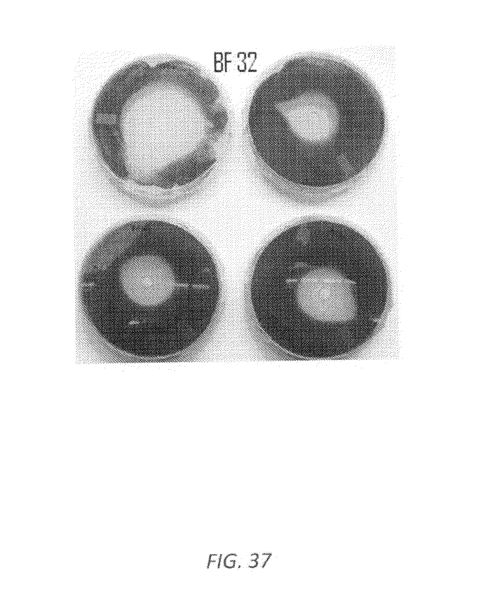

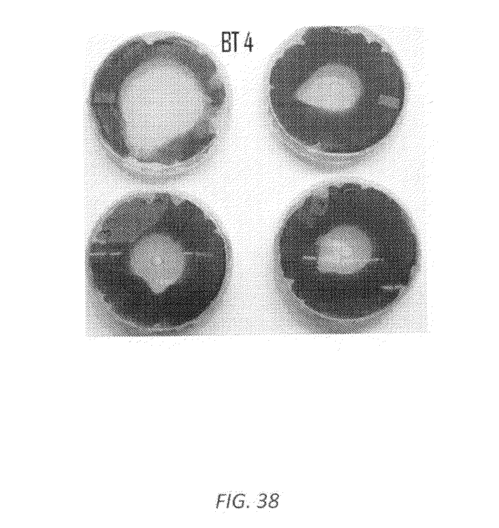

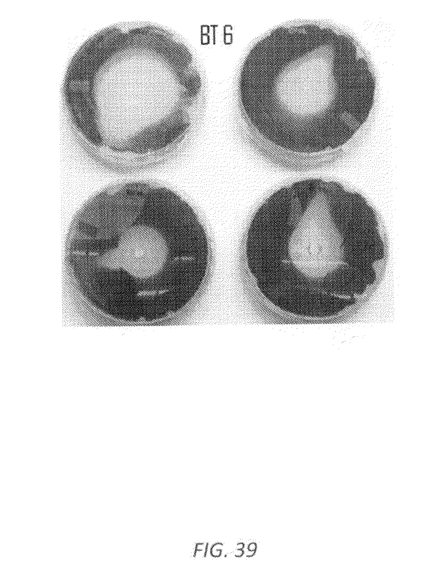

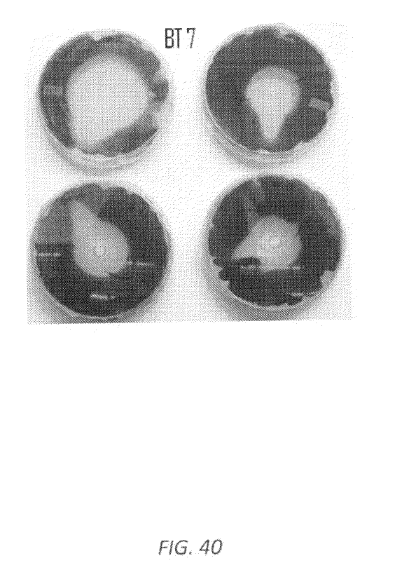

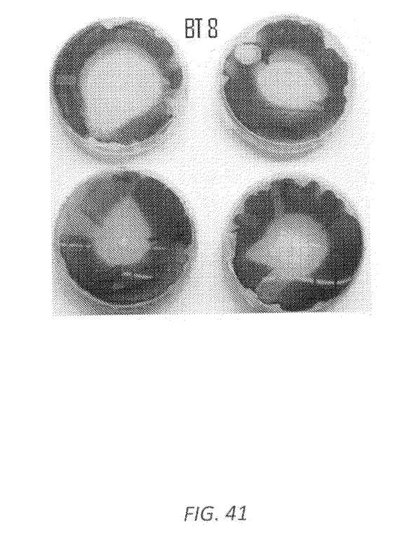

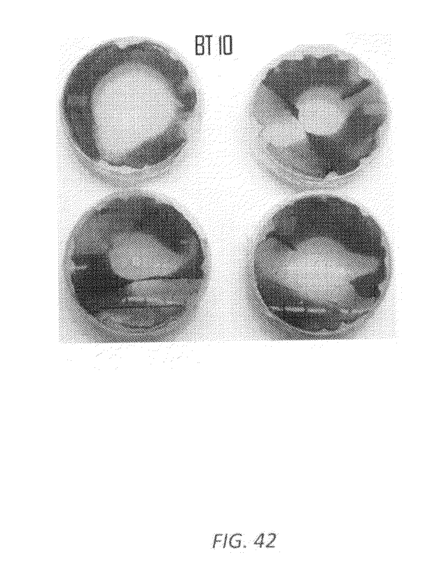

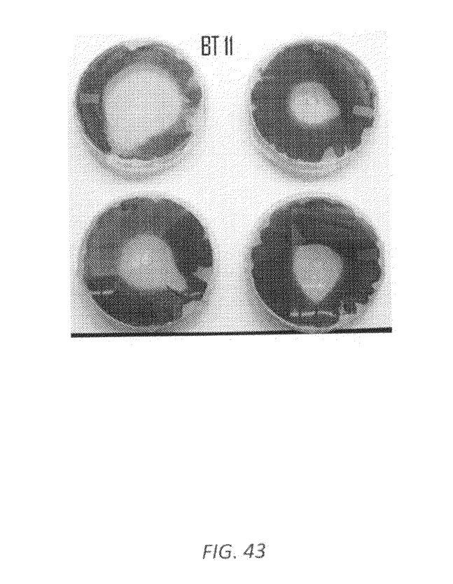

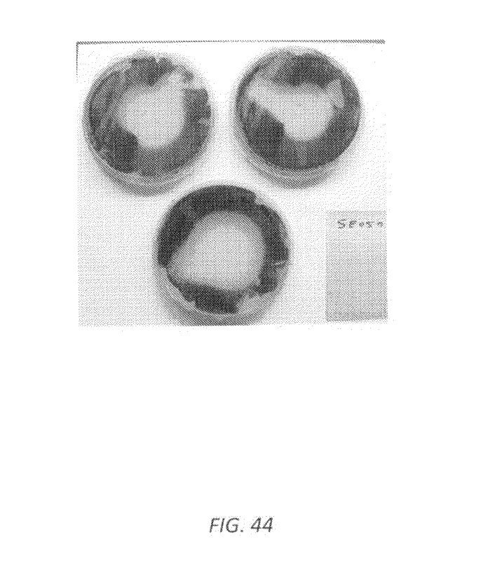

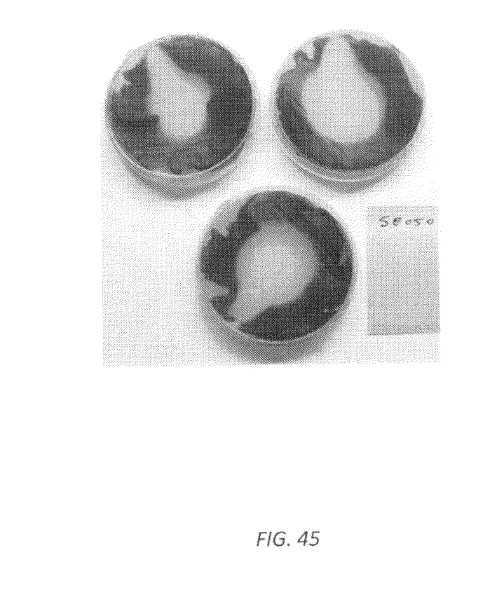

Wild-type cells average a 15.25 cm² circle of inhibition, while the sodium hypochlorite tolerant candidates range from 6.16-11.23 cm². FIG. 4 shows several representative plates from the sodium hypochlorite radial diffusion assay. FIG. 25 to FIG. 45 show additional exemplary plates from the sodium hypochlorite radial diffusion assay for the candidates.

TABLE 3
Average [cm] diameter

| | Plate 1 | Plate 2 | Plate 3 |
|---|---|---|---|
| WT | 3.75 | 4.80 | 4.60 |
| BF02 | 3.10 | 2.70 | 3.15 |
| BF03 | 3.15 | 3.60 | 3.40 |
| BF04 | 3.30 | 3.00 | 3.15 |
| BF08 | 3.30 | 3.10 | 3.15 |
| BF10/26 | 3.30 | 3.50 | 3.20 |
| BF19 | 2.90 | 3.15 | 2.75 |
| BF20 | 3.80 | 3.30 | 3.85 |
| BF21 | 2.95 | 2.90 | 2.95 |
| BF22 | 3.65 | 3.40 | 3.90 |
| BF27 | 3.10 | 3.45 | 3.15 |
| BF29 | 2.80 | 2.85 | 2.75 |
| BF30 | 3.05 | 3.10 | 3.50 |
| BF32 | 3.15 | 3.20 | 3.20 |
| BT04 | 3.10 | 3.10 | 3.40 |
| BT06 | 3.70 | 3.30 | 3.10 |
| BT07 | 3.20 | 3.45 | 3.40 |
| BT08 | 4.00 | 3.30 | 4.00 |
| BT10 | 4.10 | 3.30 | 3.30 |
| BT11 | 2.90 | 3.30 | 3.40 |

TABLE 4
Area of inhibition (cm²)

| candidate | plate 1 | plate 2 | plate 3 | average | standard dev. |
|---|---|---|---|---|---|
| WT | 11.04 | 18.10 | 16.62 | 15.25 | 3.72 |
| BF02 | 7.55 | 5.73 | 7.79 | 7.02 | 1.13 |
| BF03 | 7.79 | 10.18 | 9.08 | 9.02 | 1.19 |
| BF04 | 8.55 | 7.07 | 7.79 | 7.80 | 0.74 |
| BF08 | 8.55 | 7.55 | 7.79 | 7.96 | 0.52 |
| BF10/26 | 8.55 | 9.62 | 8.04 | 8.74 | 0.81 |
| BF19 | 6.61 | 7.79 | 5.94 | 6.78 | 0.94 |
| BF20 | 11.34 | 8.55 | 11.64 | 10.51 | 1.70 |
| BF21 | 6.83 | 6.61 | 6.83 | 6.76 | 0.13 |
| BF22 | 10.46 | 9.08 | 11.95 | 10.50 | 1.43 |
| BF27 | 7.55 | 9.35 | 7.79 | 8.23 | 0.98 |
| BF29 | 6.16 | 6.38 | 5.94 | 6.16 | 0.22 |
| BF30 | 7.31 | 7.55 | 9.62 | 8.16 | 1.27 |
| BF32 | 7.79 | 8.04 | 8.04 | 7.96 | 0.14 |
| BT04 | 7.55 | 7.55 | 9.08 | 8.06 | 0.88 |
| BT06 | 10.75 | 8.55 | 7.55 | 8.95 | 1.64 |
| BT07 | 8.04 | 9.35 | 9.08 | 8.82 | 0.69 |
| BT08 | 12.57 | 8.55 | 12.57 | 11.23 | 2.32 |
| BT10 | 13.20 | 8.55 | 8.55 | 10.10 | 2.68 |
| BT11 | 6.61 | 8.55 | 9.08 | 8.08 | 1.30 |

How to Measure an Increase in Growth of a Cell Line.

This section describes exemplary methods that can be used to determine an increase in the growth of a cell line.

An increase in the growth of a cell line can be measured by a competition assay, growth rate, carrying capacity, measuring culture productivity, cell proliferation, seed yield, organ growth, or polysome accumulation. These types of measurements are known to one of skill in the art.

The growth of the organism can be measured by optical density, dry weight, by total organic carbon, or by other methods known to one of skill in the art. These measurements can be, for example, fit to a growth curve to determine the maximal growth rate, the carrying capacity, and the culture productivity (for example, g/m2/day; a measurement of biomass produced per unit area/volume per unit time). These values can be compared to an untransformed cell line or another transformed cell line, to calculate the increase in growth in the over expressing cell line of interest.

Carrying capacity can be measured, for example, as grams per liter, grams per meter cubed, grams per meter squared, or kilograms per acre. One of skill in the art would be able to choose the most appropriate units. Any mass per unit of volume or area can be measured.

Culture productivity can be measured, for example, as grams per meter squared per day, grams per liter per day, kilograms per acre per day, or grams per meter cubed per day. One of skill in the art would be able to choose the most appropriate units.

Growth rate can be measured, for example, as per hour, per day, per generation or per week. One of skill in the art would be able to choose the most appropriate units. Any per emit time can be measured.

It is to be understood that the present invention has been described in detail by way of illustration and example in order to acquaint others skilled in the art with the invention, its principles, and its practical application. Particular compositions and processes of the present invention am not limited to the descriptions of the specific embodiments presented, but rather the descriptions and examples should be viewed in terms of the claims that follow and their equivalents.

It is to be further understood that the specific embodiments set forth herein are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the scope of the following claims.

1-111. (canceled)

112. A modified non-vascular photosynthetic comprising an organism transformed with a polynucleotide sequence comprising:
a nucleic acid sequence encoding an amino acid sequence comprising one of SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77;
wherein said organism is tolerant to sodium hypochlorite at a concentration of from 0.01 ppm to 1 ppm, from 1 ppm to 5 ppm, from 5 ppm to 10 ppm, from 10 ppm to 15 ppm, from 15 ppm to 20 ppm, from 20 ppm to 25 ppm, from 25 ppm to 30 ppm, from 30 ppm to 35 ppm, from 35 ppm to 40 ppm, from 40 ppm to 45 ppm, from 45 ppm to 50 ppm, from 50 ppm to 55 ppm, from 55 ppm to 60 ppm, from 60 ppm to 65 ppm, from 65 ppm to 70 ppm, from 70 ppm to 75 ppm, from 75 ppm to 80 ppm, from 80 ppm to 85 ppm, from 85 ppm to 90 ppm, from 90 ppm to 95 ppm, from 95 ppm to 100 ppm, from 100 ppm to 200 ppm, from 200 ppm to 500 ppm, from 500 ppm to 1000 ppm, from 1000 ppm to 2000 ppm, from 2000 ppm to 5000 ppm, from 5000 ppm to 10000 ppm, or from 10000 ppm to 15000 ppm as compared to an unmodified organism of the same species.

113. The organism of claim 112, wherein said nucleic acid sequence comprises one of
a) SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or
b) a nucleotide sequence with at least 90%, at least 95%, at least 98%, or at least 99% sequence identity to the nucleic acid sequence of SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74.

114. The modified organism of claim 112, wherein the organism is an alga or a bacterium.

115. The modified organism of claim 114, wherein the alga is a microalga.

116. The modified organism of claim 114, wherein the bacterium is a cyanobacterium.

117. The modified organism of claim 115, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

118. The modified organism of claim 114, wherein the microalga is at least one of Chlamydomonas reinhardtii, Nannochloropisis oceanica, Nannochloropsis salina, Dunaliella salina, Haematococcus pluvalis, Scenedesmus dimorphus, Dunaliella viridis, Nannochloropsis oculata, Dunaliella tertiolecta, Scenedesmus maximus, or Arthrospira fusiformus.

119. A modified non-vascular photosynthetic comprising an organism transformed with a polynucleotide sequence comprising a nucleic acid sequence encoding an amino acid sequence comprising one of SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77; wherein said modified organism has an increased growth rate as compared to an untransformed organism.

120. The modified organism of claim 119, wherein said nucleotide sequence comprises one of:
a) a nucleic acid sequence of SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74; or
b) a nucleotide sequence with at least 90%, at least 95%, at least 98%, or at least 99% sequence identity to the nucleic acid sequence of SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74.

121. The modified organism of claim 119, wherein growth is measured by carrying capacity, culture productivity, or growth rate.

122. The modified organism of claim 119, wherein the growth of the transformed organism is from 0.01% to 1% greater than the untransformed organism or the second transformed organism.

123. The modified organism of claim 119, wherein the growth of the transformed organism is from 1% to 10% greater than the untransformed organism or the second transformed organism.

124. The modified organism of claim 119, wherein the growth of the transformed organism is from 10% to 20% greater than the untransformed organism or the second transformed organism.

125. The modified organism of claim 119, wherein the growth of the transformed organism is from 20% to 40% greater than the untransformed organism or the second transformed organism.

126. The modified organism of claim 119, wherein the growth of the transformed organism is from 40% to 60% greater than the untransformed organism or the second transformed organism.

127. The modified organism of claim 119, wherein the growth of the transformed organism is from 60% to 80% greater than the untransformed organism or the second transformed organism.

128. The modified organism of claim 119, wherein the growth of the transformed organism is from 80% to 100% greater than the untransformed organism or the second transformed organism.

129. The modified organism of claim 119, wherein the growth of the transformed organism is from 100% to 200% greater than the untransformed organism or the second transformed organism.

130. The modified organism of claim 119, wherein the growth of the transformed organism is from 200% to 300% greater than the untransformed organism or the second transformed organism.

131. The modified organism of claim 119, wherein the growth of the transformed organism is from 300% to 400% greater than the untransformed organism or the second transformed organism.

132. The modified organism of claim 119, wherein the organism is grown in an aqueous environment.

133. The modified organism of claim 132, wherein the concentration of sodium hypochlorite in the aqueous environment is from 1 ppm to 5,000 ppm.

134. The modified organism of claim 132, wherein the concentration of sodium hypochlorite in the aqueous environment is from 2 ppm to 60 ppm.

135. The transformed organism of claim 119, wherein the organism is an alga or a bacterium.

136. The transformed organism of claim 135, wherein the alga is a microalga.

137. The transformed organism of claim 135, wherein the bacterium is a cyanobacterium.

138. The transformed organism of claim 135, wherein the organism is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

139. The transformed organism of claim 138, wherein the organism is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus.

140. A modified higher plant comprising a higher plant transformed with a polynucleotide sequence comprising a nucleic acid sequence encoding an amino acid sequence comprising one of SEQ ID NO: 43, SEQ ID NO: 45, SEQ ID NO: 47, SEQ ID NO: 53, SEQ ID NO: 55, SEQ ID NO: 57, SEQ ID NO: 59, SEQ ID NO: 61, SEQ ID NO: 65, SEQ ID NO: 67, SEQ ID NO: 69, SEQ ID NO: 71, SEQ ID NO: 73, SEQ ID NO: 75, or SEQ ID NO: 77: wherein said modified organism has an increased growth rate as compared to an untransformed higher plant.

141. The modified higher plant of claim 140, wherein said nucleotide sequence comprises one of:
a) a nucleic acid sequence of SEQ ID NO: 42, SEQ ID NO: 44, SEQ ID NO: 46, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74: or
b) a nucleotide sequence with at least 90%, at least 95%, at least 98%, or at least 99% sequence identity to the nucleic acid sequence of SEQ ID NO: 42, SEQ ID NO: 44, SEQ ID NO: 46, SEQ ID NO: 52, SEQ ID NO: 54, SEQ ID NO: 56, SEQ ID NO: 58, SEQ ID NO: 60, SEQ ID NO: 64, SEQ ID NO: 66, SEQ ID NO: 68, SEQ ID NO: 70, SEQ ID NO: 72, or SEQ ID NO: 74.

142. The transformed higher plant of claim 140, wherein growth is measured by carrying capacity, culture productivity, or growth rate.

143. The transformed higher plant of claim 140, wherein the growth of the transformed higher plant is from 1% to 10% greater than the untransformed higher plant.

144. The transformed higher plant of claim 140, wherein the growth of the transformed higher plant is from 10% to 20% greater than the untransformed higher plant.

145. The transformed higher plant of claim 140, wherein the growth of the transformed higher plant is from 20% to 40% greater than the untransformed higher plant.

146. The transformed higher plant of claim 140, wherein the growth of the transformed higher plant is from 40% to 60% greater than the untransformed higher plant.

147. The transformed higher plant of claim 140, wherein the growth of the transformed higher plant is from 60% to 80% greater than the untransformed higher plant.

148. The transformed higher plant of claim 140, wherein the growth of the transformed higher plant is from 80% to 100% greater than the untransformed higher plant.

149. The transformed higher plant of claim 140, wherein the transformed higher plant is a Brassica, Glycine, Gossypium, Medicago, Zea, Sorghum, Oryza, Triticum, or Panicum species.
